(19) United States
(12) Patent Application Publication
(10) Pub. No.: US 2023/0256023 A1
(43) Pub. Date: Aug. 17, 2023

(54) DERIVED MULTIPLE ALLOGENEIC PROTEINS PARACRINE SIGNALING (d-MAPPS) REGENERATIVE BIOLOGICS PLATFORM TECHNOLOGY ADJUVANT THERAPY FOR THE PREVENTION AND TARGETED TREATMENT OF CANCER AND OTHER DISORDERS

(71) Applicant: MAM Holdings of West Florida, L.L.C., Palm Harbor, FL (US)

(72) Inventor: Carl Randall Harrell, Tarpon Springs, FL (US)

(21) Appl. No.: 18/110,317

(22) Filed: Feb. 15, 2023

Related U.S. Application Data
(60) Provisional application No. 63/268,137, filed on Feb. 17, 2022.

(51) Int. Cl. A61K 35/28; A61P 35/00; A61K 45/06

(52) U.S. Cl. CPC A61K 35/28; A61P 35/00; A61K 45/06

(57) ABSTRACT

Methods of preventing and treating cancer, and of suppressing the growth or proliferation of cancer, using the amniotic fluid-derived d-MAPPS™ regenerative biologics platform technology (“d-MAPPS”). The d-MAPPS compositions contain significant numbers of placental tissue-derived mesenchymal stem cells, growth factors, anti-inflammatory cytokines, and are amenable for long-term storage without the loss of biological potency. In certain embodiments, d-MAPPS is shown to improve survival of tumor bearing animals. In other embodiments, d-MAPPS is used in combination with, or formulated with, one or more additional active agents.

### FIELD

The present invention relates generally to methods and compositions for immunotherapy. In particular, the present invention relates to the use of d-MAPPS™ regenerative biologics platform technology as a targeted adjuvant therapy for the prevention and treatment of cancers, tumors, and various associated disorders. The compositions described herein may be used in combination with, or formulated with, one or more additional active agents.

### BACKGROUND

During the last three decades, immunosuppressive drugs have been frequently used in clinical practice due to the increase of autoimmune and inflammatory diseases. However, long-term use of immunosuppressive agents may result in the development of severe infections due to the inhibition of anti-microbial immune response. Various studies are focused on the development of novel immunomodulatory compounds that inhibit detrimental immune responses without causing life-threatening immunosuppression. This is particularly true in the field of cancer immunotherapy.

Mesenchymal stem cells (“MSC”) have potent immunosuppressive properties and their therapeutic potential in the alleviation of autoimmune diseases and select cancers have been demonstrated in experimental and clinical trials. Amniotic fluid-derived MSCs (“AF-MSCs”) exhibit an increased proliferation rate and greater immunosuppressive potential than bone marrow derived MSCs. Both AF-MSCs and placental tissue-derived mesenchymal stem cells (“PL-MSCs”) contain a variety of biological factors including carbohydrates, proteins and peptides, lipids, lactate, pyruvate, electrolytes, enzymes, and hormones. In addition, AF-MSCs and PL-MSCs contain many important growth factors including epidermal growth factor (“EGF”), transforming growth factor alpha (“TGF-α”), transforming growth factor beta-1, insulin-like growth factor I (“IGF-I”), and erythropoietin (“EPO”).

AF-MSCs are believed to have antitumor effects and are preferred for their properties, such as immune-modulating capacity and ability to accumulate at the tumor site. Furthermore, AF-MSCs are known to have a scalable capacity for the mass production of exosomes and other immunomodulatory cells for drug delivery. Apart from regulating tumor cell fate, AF-MSC-derived exosomes are capable of being applied for delivery of anticancer therapeutics. AF-MSCs-derived biological products present numerous benefits as therapeutic agents compared to cells or synthetic nanoparticles including the potential to be engineered and exceptional biocompatibility/stability features.

In line with these observations, the present inventors have recently designed an AF-MSC-derived biological product d-MAPPS™ regenerative biologics platform technology (“d-MAPPS”), which contains large numbers of AF-MSC-sourced growth factors, anti-inflammatory cytokines and chemokines. Notably, the acronym d-MAPPS in d-MAPPS™ regenerative biologics platform technology stands for “derived Multiple Allogeneic Proteins Paracrine Signaling”. In vitro, d-MAPPS™ regenerative biologics platform technology efficiently inhibits proliferation of activated human peripheral blood mononuclear cells (“pbMNCs”). d-MAPPS™ regenerative biologics platform technology further suppresses production of inflammatory cytokines and promotes secretion of immunosuppressive factors in pbMNCs. In addition, d-MAPPS™ regenerative biologics platform technology favors development of tolerogenic and regulatory phenotype in activated monocytes and lymphocytes, indicating its potential for therapeutic use in the alleviation of various cancers. While the potential for therapeutic use of AF-MSCs-derived biological products (e.g., d-MAPPS) in cancers is apparent, evidence of improved survival of tumor bearing animals is needed to bring this exciting development a step closer to clinical reality.

### BRIEF SUMMARY

In some embodiments, the present inventors disclose a method for prevention and treatment of cancers and tumors in a subject, comprising administering to the subject an effective amount of d-MAPPS™ regenerative biologics platform technology (“d-MAPPS”). Importantly, d-MAPPS™ regenerative biologics platform technology includes immunostimulatory molecules (e.g., IL-27 and CXCL16) that enhance T-cell driven immune responses in a tumor microenvironment. In some embodiments, a method for prevention and treatment of cancers is disclosed including altering the response of endogenous immune cells in the subject provided, comprising administering to the subject an effective amount of d-MAPPS™ regenerative biologics platform technology, thereby altering the response of endogenous immune cells (e.g., dendritic cells, macrophages, natural killer cells, T cells, and the like) in the subject. In embodiments, administration of an effective amount of d-MAPPS™ regenerative biologics platform technology increases the likelihood of survival of the subject and decreases the incidence of cancers and/or tumors in the subject. Further, administering d-MAPPS reduces tumor weight and/or tumor volume in a subject with cancer. In some embodiments, d-MAPPS™ regenerative biologics platform technology may be administered in combination with one or more agents selected from the group consisting of d-MAPPS-associated MSCs, placenta tissue-derived MSCs, antimicrobial agents, analgesic agents, local anesthetic agents, anti-inflammatory agents, anti-oxidant agents, immunosuppressant agents, anti-allergenic agents, enzyme cofactors, essential nutrients, growth factors, and combinations thereof.

In some embodiments, a pharmaceutical composition comprising d-MAPPS™ regenerative biologics platform technology (also referred to herein as “d-MAPPS” and “d-MAPPS pharmaceutical composition”) and one or more pharmaceutically acceptable excipients is provided. Said pharmaceutical composition may comprise one or more agents selected from the group consisting of adjuvants, antioxidants, anti-inflammatory agents, growth factors, neuroprotective agents, antimicrobial agents, local anesthetics, and combinations thereof. In addition, said d-MAPPS pharmaceutical composition may comprise exosomes generated ex vivo from mesenchymal stem cells, wherein the mesenchymal stem cells are placental tissue-derived mesenchymal stem cells.

### BRIEF DESCRIPTION OF THE DRAWINGS

FIG. 1A shows that the mean value of time period in days from inoculation of tumor cells to the appearance of palpable primary tumor in 4T1+d-MAPPS+-treated mice was significantly longer than in 4T1+saline-treated animals. FIG. 1B illustrates that d-MAPPS™ regenerative biologics platform technology (“d-MAPPS”) prevented development of breast cancer in the majority of 4T1-treated mice. Specifically, 43.75% of 4T1+d-MAPPStreated mice (7 out of 16) developed tumor while all (100%) of 4T1+saline-treated mice developed breast cancer. FIG. 1C shows that lung and liver metastatic colonies were determined in all 4T1+saline-treated mice and in 4T1+d-MAPPS treated mice that developed tumors (upper and middle panels), while brain metastasis were not found either in 4T1+saline or 4T1+d-MAPPstreated tumor bearing mice (lower panels). FIG. 1D shows that the mean value of primary tumor volume in 4T1+saline-treated mice was significantly higher than in 4T1+d-MAPPStreated mice. Similarly, FIG. 1E illustrates that primary tumor weight was significantly higher in saline-treated animals compared to d-MAPPS treated tumor bearing animals.

FIG. 2A shows that d-MAPPS™ regenerative biologics platform technology (“d-MAPPS”) significantly increases serum levels of anti-tumorigenic chemokines and cytokines CXCL16 (FIG. 2A), IL-27 (FIG. 2B), IFN-γ (FIG. 2C), IL-17 (FIG. 2D)) and down-regulates concentrations of immunosuppressive cytokines TGF-β (FIG. 2E) and IL-10 (FIG. 2F)) in mice with established mammary tumors. Analogously, increased concentration of CXCL16, IL-27, IFN-γ and IL-17 and decreased concentration of TGF-3 and IL-10 were measured in the tumors of 4T1+d-MAPPS treated mice (FIG. 2G).

FIG. 3 shows that d-MAPPS™ regenerative biologics platform technology (“d-MAPPS”) did not significantly alter phenotype and function of NK cells and macrophages. Specifically, there was no significant difference in the total number of tumor infiltrated, cytotoxic CD178 and Granzyme B-expressing, IFN-γ or IL-17-producing NK1.1+NK cells (FIG. 3A-D) and in the total number of IL-12-, TNF-α and IL-10-producing, CD80, CD86 and I-A-expressing F4/80+TAMs (FIG. 3E-J) between 4T1+saline and 4T1+d-MAPPS treated mice.

FIG. 4 shows that d-MAPPS significantly enhances antigen-presenting properties of tumor infiltrated DCs, including higher number of DCs that express MHC class II molecule (e.g., CD11c+I-A+TNF-α) and produce TNF-α (FIG. 4A-B), higher percentage of DCs that express co-stimulatory CD80 and CD86 molecules (FIG. 4C), and higher percentage of DCs that express IL-12 (FIG. 4D). Additionally, d-MAPPS reduced percentage of tolerogenic, IL-10-producing DCs in the tumors (FIG. 4E), preventing tumor cell-driven generation of immunosuppressive microenvironment.

FIG. 5 shows that a significantly higher number of anti-tumorigenic CD4+Th1 and Th17 cells were observed in the mammary cancers of 4T1+d-MAPPS treated mice (FIG. 5A-C). Total numbers of IFN-γ-producing Th1 and IL-17-producing Th17 cells were increased in the breast tumors of 4T1+d-MAPPS treated animals (FIG. 5A-C) while there was no significant difference in total number of CD4+IL-4+Th2 cells between the tumors of 4T1+saline and 4T1+d-MAPPS treated mice (FIG. 5D), confirming that d-MAPPS™ regenerative biologics platform technology (“d-MAPPS”) favored generation of anti-tumorigenic Th1 and Th17 immune response. Further, d-MAPPS inhibits generation of immunosuppressive phenotype in tumor-infiltrated CD4+ T cells (FIG. 5E).

FIG. 6 shows that d-MAPPS also significantly increases the presence of tumor-infiltrated cytotoxic CD178-expressing and Granzyme B-producing cytotoxic CD8+T cells (see CTLs; FIG. 6A-B). Additionally, d-MAPPS favors activation and expansion of tumoricidal IFN-γ-producing and IL-17-producing CD8+ CTLs (FIG. 6C-D) and inhibits generation of immunosuppressive and pro-tumorigenic IL-10-producing and FoxP3-expressing CD8+T cells (FIG. 6E-F).

FIG. 7A is a line graph showing a more than fifty percent decrease in the incidence of cancers for the d-MAPPS experimental group in a 4T1 breast cancer model (BALBc mice injected with 4T1 mammary carcinoma cells). Mice were randomly divided into two groups to receive either saline or d-MAPPS™ regenerative biologics platform technology (“d-MAPPS”) treatment which was intraperitoneally injected every day for days 1-36. FIG. 7B is a line graph showing improved survival of d-MAPPS-treated mice relative to saline-treated mice in a 4T1 breast cancer model. Mice were divided into two groups to receive either saline or d-MAPPS treatment, which was intraperitoneally injected every day for days 1-36.

FIG. 8A is a bar graph showing that d-MAPPS significantly reduces tumor weight in mice that developed tumors. FIG. 8B is a bar graph showing that d-MAPPS significantly reduces tumor volume in mice that developed tumors.

FIG. 9 is a bar graph showing that d-MAPPS treatment significantly decreases serum levels of TNF-alpha in a 4T1 breast cancer model. Mice were intraperitoneally injected every day for days 36 days, and were divided into four groups: saline-treated 4T1 mice, d-MAPPS-treated 4T1 mice, saline-treated control mice, and d-MAPPS-treated control mice. Control mice represented are represented by BALBc mice that were not injected with 4T1 mammary carcinoma cells.

FIG. 10 is a bar graph showing that d-MAPPS™ regenerative biologics platform technology (“d-MAPPS”) treatment significantly increases serum levels of anti-tumorigenic IFN-gamma in a 4T1 breast cancer model. Mice were intraperitoneally injected every day for days 36 days and were divided into four groups: saline-treated 4T1 mice, d-MAPPS-treated 4T1 mice, saline-treated control mice, and d-MAPPS-treated control mice.

FIG. 11 is a bar graph showing that d-MAPPS treatment significantly increases serum levels of IL-17 in a 4T1 breast cancer model. Mice were intraperitoneally injected every day for days 36 days and were divided into four groups: saline-treated 4T1 mice, d-MAPPS-treated 4T1 mice, saline-treated control mice, and d-MAPPS-treated control mice.

FIG. 12 is a bar graph showing that d-MAPPS treatment significantly decreases serum levels of TGF-beta in a 4T1 breast cancer model. Mice were intraperitoneally injected every day for days 36 days and were divided into four groups: saline-treated 4T1 mice, d-MAPPS-treated 4T1 mice, saline-treated control mice, and d-MAPPS-treated control mice.

FIG. 13 is a bar graph showing that d-MAPPS treatment significantly decreases serum levels of IL-10 in a 4T1 breast cancer model. Mice were intraperitoneally injected every day for days 36 days and were divided into four groups: saline-treated 4T1 mice, d-MAPPS-treated 4T1 mice, saline-treated control mice, and d-MAPPS-treated control mice.

### DESCRIPTION

Unless otherwise noted, technical terms are used according to conventional usage. Aspects of the disclosed methods employ, unless indicated specifically to the contrary, conventional methods of chemistry, biochemistry, organic chemistry, molecular biology, microbiology, recombinant DNA techniques, genetics, immunology, and cell biology, many of which are described below for the purpose of illustration. Such techniques are explained fully in the literature. Definitions of common terms in molecular biology may be found in Lewin's Genes X, ed. Krebs et al., Jones and Bartlett Publishers, 2009 (ISBN 0763766321); George P. Rédei, Encyclopedic Dictionary of Genetics, Genomics, Proteomics and Informatics, 3^{rd} Edition, Springer, 2008 (ISBN: 1402067534); Ausubel et al., Current Protocols in Molecular Biology (John Wiley and Sons, updated July 2008); Short Protocols in Molecular Biology: A Compendium of Methods from Current Protocols in Molecular Biology, Greene Pub. Associates and Wiley-Interscience; Glover, DNA Cloning: A Practical Approach, vol. I & II (IRL Press, Oxford, 1985); Anand, Techniques for the Analysis of Complex Genomes, (Academic Press, New York, 1992); Transcription and Translation (B. Hames & S. Higgins, Eds., 1984); Perbal, A Practical Guide to Molecular Cloning (1984); Kendrew et al. (eds.), The Encyclopedia of Molecular Biology, published by Blackwell Publishers, 1994 (ISBN 0632021829); Robert A. Meyers (ed.), Molecular Biology and Biotechnology: a Comprehensive Desk Reference, published by Wiley, John & Sons, Inc., 1995 (ISBN 0471186341); Harlow and Lane, Antibodies, (Cold Spring Harbor Laboratory Press, Cold Spring Harbor, N.Y., 1998) Current Protocols in Immunology Q. E. Coligan, A. M. Kruisbeek, D. H. Margulies, E. M. Shevach and W. Strober, eds., 1991); Annual Review of Immunology; as well as monographs in journals such as Advances in Immunology, and other similar references.

### I. Definitions

As used herein, the singular forms “a,” “an,” and “the,” may refer to both the singular as well as plural, unless the context clearly indicates otherwise. As used herein, the term “comprises” can mean “includes.” Thus, “comprising a cancer-associated immune cell” may mean “including a cancer-associated immune cell” without excluding other elements. Although many methods and materials similar or equivalent to those described herein can be used, particular suitable methods and materials are described below. In case of conflict, the present specification, including explanations of terms, will control. In addition, the materials, methods, and examples are illustrative only and not intended to be limiting. All references, including patent applications and patents are herein incorporated by reference.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present disclosure. Thus, the appearances of the foregoing phrases in various places throughout this specification are not necessarily all referring to the same embodiment.

The term “active agent,” refers to a physiologically or pharmacologically active substance that acts locally and/or systemically in the body. An active agent is a substance that is administered to an individual for the treatment (e.g., therapeutic agent, cancer therapeutic agent, and the like), prevention (e.g., prophylactic agent), or diagnosis (e.g., diagnostic agent) of a disease or disorder. Active agents may also include therapeutics that prevent or alleviate symptoms such as symptoms associated with breast cancer or related treatments.

The term “administering” or “administration” refers to providing or giving a subject an agent or formulation, such as d-MAPPS or another cancer prophylactic or anti-cancer therapeutic agent, by any effective route. Exemplary routes of administration include, but are not limited to, injection (such as subcutaneous, subdermal, intramuscular, intradermal, intraperitoneal, intracerebroventricular, intraosseous, intratumoral, intraprostatic, and intravenous), transdermal, intranasal, oral, vaginal, rectal, and inhalation routes.

The term “amniotic factor,” generally refers to molecules naturally present in the amniotic fluid. These include carbohydrates, proteins and peptides such as enzymes and hormones, lipids, metabolic substrates and products such as lactate and pyruvate, and electrolytes.

The term “antigen” refers to a compound, composition, or substance that can stimulate the production of antibodies or an immune response in an animal, including compositions (such as one that includes a tumor-specific protein) that are injected or absorbed into an animal. An antigen reacts with the products of specific humoral or cellular immunity, including those induced by heterologous antigens, such as the disclosed antigens.

The term “cancer” refers to a class of diseases or conditions in which abnormal cells divide without control and can invade nearby tissues. A malignant cancer is one in which a group of tumor cells display one or more of uncontrolled growth (e.g., division beyond normal limits), invasion (e.g., intrusion on and destruction of adjacent tissues), and metastasis (e.g., spread to other locations in the body via lymph or blood). As used herein, the term “metastasize” refers to the spread of cancer from one part of the body to another. A tumor formed by cells that have spread is called a “metastatic tumor” or a “metastasis.” The metastatic tumor contains cells that are like those in the original (primary) tumor. A “cancer cell” or “tumor cell” refers to an individual cell of a cancerous growth or tissue. A tumor refers generally to a swelling or lesion formed by an abnormal growth of cells, which may be benign, pre-malignant, or malignant. Most cancers form tumors, but some, e.g., leukemia, and some blood cancers, do not necessarily form tumors. For those cancers that form tumors, the terms cancer (cell) and tumor (cell) are used interchangeably. The amount of a tumor in an individual is the “tumor burden” which can be measured as the number, volume, or weight of the tumor.

Exemplary tumors, such as “cancers”, that can be treated using the disclosed d-MAPPS formulations include solid tumors, such as breast carcinomas (e.g. lobular and duct carcinomas, such as a triple negative breast cancer), sarcomas, carcinomas of the lung (e.g., non-small cell carcinoma, large cell carcinoma, blood cancers, squamous carcinoma, and adenocarcinoma), mesothelioma of the lung, colorectal adenocarcinoma, stomach carcinoma, prostatic adenocarcinoma, ovarian carcinoma (such as serous cystadenocarcinoma and mucinous cystadenocarcinoma), ovarian germ cell tumors, testicular carcinomas and germ cell tumors, pancreatic adenocarcinoma, biliary adenocarcinoma, hepatocellular carcinoma, bladder carcinoma (including, for instance, transitional cell carcinoma, adenocarcinoma, and squamous carcinoma), renal cell adenocarcinoma, endometrial carcinomas (including, e.g., adenocarcinomas and mixed Mullerian tumors (carcinosarcomas)), carcinomas of the endocervix, ectocervix, and vagina (such as adenocarcinoma and squamous carcinoma of each of same), tumors of the skin (e.g., squamous cell carcinoma, basal cell carcinoma, malignant melanoma, skin appendage tumors, Kaposi sarcoma, cutaneous lymphoma, skin adnexal tumors and various types of sarcomas and Merkel cell carcinoma), esophageal carcinoma, carcinomas of the nasopharynx and oropharynx (including squamous carcinoma and adenocarcinomas of same), salivary gland carcinomas, brain and central nervous system tumors (including, for example, tumors of glial, neuronal, and meningeal origin), tumors of peripheral nerve, soft tissue sarcomas and sarcomas of bone and cartilage, head and neck squamous cell carcinoma, and lymphatic tumors (including B-cell and T-cell malignant lymphoma). In one example, the tumor is an adenocarcinoma. In another example, the cancer is pancreatic adenocarcinoma. In yet another example, the cancer is colorectal adenocarcinoma. The disclosed d-MAPPS formulations can also be used to treat liquid tumors, such as a lymphatic, white blood cell, or other type of leukemia. In a specific example, the tumor treated is a tumor of the blood, such as a leukemia (for example acute lymphoblastic leukemia (ALL), chronic lymphocytic leukemia (CLL), acute myelogenous leukemia (AML), chronic myelogenous leukemia (CML), hairy cell leukemia (HCL), T-cell prolymphocytic leukemia (T-PLL), large granular lymphocytic leukemia, and adult T-cell leukemia), a lymphoma (such as Hodgkin's lymphoma or non-Hodgkin's lymphoma), or a myeloma.

The term “combination therapy” refers to administration of different agents or therapies in a sequential or simultaneous manner. The individual elements may be administered at different times and/or by different routes but act in combination to provide a beneficial effect.

The term “decrease/lower/lessen/reduce/abate” refers generally to the ability of a composition contemplated herein (e.g., anti-cancer d-MAPPS) to produce, elicit, or cause a lesser physiological response (i.e., downstream effects) compared to the response caused by either vehicle or a control molecule/composition. A “decrease” or “reduced” amount is typically a “statistically significant” amount, and may include a decrease that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) the response (reference response) produced by vehicle, a control composition, or the response in a particular cell lineage.

The term “dendritic cell” refers to a type of specialized antigen presenting cell (APC) involved in innate and adaptive immunity. Also referred to as “DC.” Dendritic cells may be present in the tumor microenvironment and these are referred to as “tumor-associated dendritic cells” or “tDCs.”

The term “effective amount/therapeutically effective amount”) refers to the amount of an agent (e.g., a d-MAPPS formulation disclosed herein, or other anti-cancer agents) that is sufficient to effect beneficial or desired therapeutic result, including clinical results. An effective amount may vary depending upon one or more of: the subject and disease condition being treated, the sex, weight and age of the subject, the severity of the disease condition, the manner of administration, the ability of the d-MAPPS formulations to elicit a desired response in the individual, and the like. The beneficial therapeutic effect can include enablement of diagnostic determinations; prevention of disease or tumor formation; amelioration of a disease, symptom, disorder, or pathological condition; reducing or preventing the onset of a disease, symptom, disorder or condition; and generally counteracting a disease, symptom, disorder or pathological condition. The term “effective amount” includes an amount that is effective to “treat” a subject (e.g., a patient or subject). When a therapeutic amount is indicated, the precise amount of the compositions of the present disclosure to be administered can be determined by a physician with consideration of individual differences in age, weight, tumor size, extent of infection or metastasis, and condition of the patient (subject).

In one embodiment, an “effective amount” (e.g., of d-MAPPS, a costimulatory molecule, or ionizing radiation) may be an amount sufficient to increase the rate of survival of a subject, reduce the volume/size of a tumor, the weight of a tumor, the number/extent of metastases, reduce the volume/size of a metastasis, the weight of a metastasis, or combinations thereof, for example by at least about 10%, at least about 20%, at least about 25%, at least about 50%, at least about 70%, at least about 75%, at least about 80%, at least about 90%, at least about 95%, or at least about 99% (as compared to no administration of the therapeutic agent). In one embodiment, an “effective amount” (e.g., of d-MAPPS, or ionizing radiation described herein) may be an amount sufficient to increase the survival time of a subject, such as a subject with cancer, for example by at least about 10%, at least about 20%, at least about 25%, at least about 50%, at least about 70%, at least about 75%, at least about 80%, at least about 90%, at least about 95%, 100%, 200%, 300%, 400%, or 500% (as compared to no administration of the therapeutic agent).

The term “enhance/induce/increase” refers generally to the ability of a composition contemplated herein to produce, elicit, or cause a greater physiological response (i.e., downstream effects) compared to the response caused by either vehicle or a control molecule/composition. A measurable physiological response may include inducing response of cancer-associated endogenous immune cells in the subject and/or an increase in cancer cell death killing ability, among others apparent from the understanding in the art and the description herein. An “enhanced” or “increased” amount is typically a “statistically significant” amount, and may include an increase that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) the response produced by vehicle or a control composition.

The term “enteral administration” means administration via absorption through the gastrointestinal tract. Enteral administration can include oral and sublingual administration, gastric administration, or rectal administration.

The term “growth factors,” refers to a group of proteins or hormones that stimulate the cellular growth. Growth factors play an important role in promoting cellular differentiation and cell division, and they occur in a wide range of organisms.

The term “immune cell” refers to any cell of the immune system that has one or more effector functions (e.g., cytotoxic cell killing activity, secretion of cytokines, induction of ADCC and/or CDC).

The terms “immunologic”, “immunological” or “immune” response is the development of a beneficial humoral (antibody mediated) and/or a cellular (mediated by antigen-specific T cells or their secretion products) response directed against an immunogen in a recipient patient. Such a response can be an active response induced by administration of immunogen or a passive response induced by administration of antibody or primed T-cells. A cellular immune response is elicited by the presentation of polypeptide epitopes in association with Class I or Class II MHC molecules to activate antigen-specific CD4+T helper cells and/or cos+ cytotoxic T cells. The response may also involve activation of monocytes, macrophages, NK cells, basophils, dendritic cells, astrocytes, microglia cells, eosinophils or other components of innate immunity. The presence of a cell-mediated immunological response can be determined by proliferation assays (CD4+ T cells) or CTL (cytotoxic T lymphocyte) assays. The relative contributions of humoral and cellular responses to the protective or therapeutic effect of an immunogen can be distinguished by separately isolating antibodies and T-cells from an immunized syngeneic animal and measuring protective or therapeutic effect in a second subject.

The term “ionizing radiation” refers to radiation, traveling as a particle or electromagnetic wave, that carries sufficient energy to detach electrons from atoms or molecules, thereby ionizing an atom or a molecule. Ionizing radiation is made up of energetic subatomic particles, ions or atoms moving at high speeds and electromagnetic waves on the high-energy end of the electromagnetic spectrum. Radiation has been demonstrated to induce adaptive immune responses to mediate tumor regression. In addition, the induction of type I IFNs by radiation is essential for the function of CD8+ T cells. Radiation induces cell stress and causes excess DNA breaks, indicating that nucleic acid-sensing pathway likely account for the induction of type I IFNs upon radiation. Type I IFN responses in DCs dictate the efficacy of antitumor radiation. In contrast, chemotherapeutic agents and anti-HER2 antibody treatments have been demonstrated to depend on a distinct immune mechanism to trigger adaptive immune responses. In general, therapeutic radiation-mediated antitumor immunity depends on a proper cytosolic DNA sensing pathway. In embodiments, d-MAPPS™ regenerative biologics platform technology is administered in combination with radiation therapy.

The term “macorphages” refers to a type of white blood cell of the immune system that engulfs and digests cellular debris, foreign substances, microbes, cancer cells, and the like. These phagocytes include various subtypes (e.g., histiocytes, Kupffer cells, alveolar macrophages, microglia, and others), but all are part of the mononuclear phagocyte system. Besides phagocytosis, they play a critical role in both innate and adaptive immunity by recruiting other endogenous immune cells such as lymphocytes. For example, they are important as antigen presenters to T cells. In humans, dysfunctional macrophages cause severe diseases such as chronic granulomatous disease that result in frequent infections. Beyond increasing inflammation and stimulating the immune system, macrophages also play an important anti-inflammatory role and can decrease immune reactions through the release of cytokines. Macrophages that encourage inflammation are called M1 macrophages, whereas those that decrease inflammation and encourage tissue repair are called M2 macrophages.

The term “parenteral administration”, means administration by any method other than through the digestive tract or non-invasive topical or regional routes. For example, parenteral administration may include administration to a patient via intravenous, intradermal, intraperitoneal, intrapleural, intratracheal, intraarticular, intrathecal, intramuscular, subcutaneous, subjunctival, injection, and/or infusion.

The term “peptide” refers to a polymer of amino acid residues. “Polypeptide,” “peptide”, and “protein” are used interchangeably herein. The terms apply to amino acid polymers of one or more amino acid residues, an artificial chemical mimetic of a corresponding naturally occurring amino acid, naturally occurring amino acid polymers, and non-naturally occurring amino acid polymers.

The term “subject/Individual/Patient” refers to a vertebrate, such as a mammal, for example a human. Mammals include, but are not limited to, murines, simians, humans, farm animals, sport animals, and pets. In one embodiment, the subject is a non-human mammalian subject, such as a monkey or other non-human primate, mouse, rat, rabbit, guinea pig, pig, goat, sheep, dog, cat, horse, or cow. In some examples, the subject has a tumor, such as a cancer, that can be treated using d-MAPPS, d-MAPPS pharmaceutical compositions, and other d-MAPPS formulations disclosed herein. In some examples, the subject is a laboratory animal/organism, such as a mouse, rabbit, guinea pig, or rat. In one example a subject includes farm animals and domestic animals or pets (such as a cat or dog). In one example, a subject is a human patient that has a cancer, has been diagnosed with a cancer, or are at risk or having a cancer. A “patient” can refers to a subject that has been diagnosed with a particular indication that can be treated with d-MAPPS pharmaceutical compositions and methods disclosed elsewhere herein.

The term “topical administration” means the non-invasive administration to the skin, orifices, or mucosa. Topical administrations can be administered locally, i.e., they are capable of providing a local effect in the region of application without systemic exposure. Topical formulations can provide systemic effect via adsorption into the blood stream of the individual. Topical administration can include, but is not limited to, cutaneous and transdermal administration, buccal administration, intranasal administration, intravaginal administration, intravesical administration, ophthalmic administration, pulmonary, and rectal administration.

The term “treating, treatment, and therapy” refers to any success or indicia of success in the attenuation or amelioration of an injury, pathology or condition, including any objective or subjective parameter such as abatement, remission, diminishing of symptoms or making the condition more tolerable to the patient, slowing in the rate of degeneration or decline, making the final point of degeneration less debilitating, improving a subject's physical or mental well-being, or prolonging the length of survival. Treatment does not necessarily indicate complete eradication or cure of the disease or condition, or associated symptoms thereof. The treatment may be assessed by objective or subjective parameters; including the results of a physical examination, blood and other clinical tests (such as imaging), and the like. In some examples, treatment with the disclosed methods results in a decrease in the number, volume, and/or weight of a tumor and/or metastases.

The term “molecular weight,” generally refers to the relative average chain length of the bulk polymer or protein, unless otherwise specified. In practice, molecular weight can be estimated or characterized using various methods including gel permeation chromatography (GPC) or capillary viscometry. GPC molecular weights are reported as the weight-average molecular weight (MW) as opposed to the number-average molecular weight (MN). Capillary viscometry provides estimates of molecular weight as the inherent viscosity determined from a dilute polymer solution using a particular set of concentration, temperature, and solvent conditions.

### II. Compositions

Formulations of purified sterilized human amniotic fluid are provided. In some embodiments, the formulations include diluted sterile de-cellularized human amniotic fluid (referred to as d-MAPPS™ regenerative biologics platform technology or “d-MAPPS”), either in fluid form or solid form, for example, lyophilized powder, alone or in combination with appropriate excipients. In some embodiments, other active agents may be co-administered with d-MAPPS including secondary anti-cancer agents, anti-inflammatories, exogenous immune cells, small molecules, therapeutic proteins, and the like. Notably, purified d-MAPPS contains over 300 human growth factors. d-MAPPS is devoid of amniotic stem cells and elements of micronized membrane or chorion particles via a series of centrifugation and filtration steps. The d-MAPPS formulations generally include exosomes generated ex vivo from mesenchymal stem cells. Typically, the de-cellularized amniotic fluid retains more than 90% of the amniotic proteins compared to the raw amniotic fluid. In some embodiments, d-MAPPS compositions retain most amniotic factors after short-term or long-term storage under temperature-controlled conditions either as a liquid or as lyophilized powder, for example, at least 60% of the total protein content compared to that of the fresh d-MAPPS, preferably more than 85%.

A. Amniotic Fluid and d-MAPPS Compositions

Amniotic fluid (“AF”) contains nutrients and growth factors that facilitate fetal growth, provides mechanical cushioning and antimicrobial effectors that protect the fetus, and allows assessment of fetal maturity and disease. AF typically contains mixtures of growth factors, pro-inflammatory cytokines and anti-inflammatory cytokines, as well as a variety of macromolecules including carbohydrates, proteins and peptides, lipids, lactate, pyruvate, electrolytes, enzymes, and hormones. Diluted sterile de-cellularized human amniotic fluid (“d-MAPPS”), retains the same amniotic factors described above.

In the preferred embodiment, the raw fluid directly collected from the source is not heat-treated, chemical-treated, fractionated to produce the disclosed formulations. In some embodiments, the formulation retains more than 50%, more than 60%, more than 70%, more than 80%, or preferably more than 90%, of the amniotic factors present in the raw fluid. In some embodiments, the formulations are not diluted with any additional solution. In other embodiments, the formulations are not concentrated relative to the raw fluid.

As described above, the AF formulation may be diluted, sterilized, and de-cellularized to produce a d-MAPPS formulation (e.g., “d-MAPPS”, or a “d-MAPPS pharmaceutical composition”). Notably, d-MAPPS contains over 300 human growth factors. Further, d-MAPPS is devoid of amniotic cells (e.g., amniotic stem cells) micronized amnion membrane, and chorion membrane particles. In some embodiments, growth factors are present at lower concentrations in d-MAPPS than in human amniotic fluid. The purified fluid is sterilized without the use of harsh terminal irradiation, e-beam or Ethylene Oxide (EO). In the preferred embodiment, the process consists of separating the stem cells from the AF using centrifugation and utilizing a series of filtration devices to remove all remaining extraneous cells. Each lot is certified sterile and certified to contain <1 ppm foreign particle.

Generally, methods of preparing d-MAPPS pharmaceutical compositions involve a series of centrifugation and filtrations steps. Relatedly, preferred methods of preparing sterile de-cellularized amniotic fluid are described in detail in U.S. application Ser. No. 15/053,497.

1. Collecting Amniotic Fluid

In some embodiments, amniotic fluid is collected in a sterile operating room environment during an elective C-section. Typically, the procedure is performed using an ultrasound device to provide guidance for the process of obtaining amniotic fluid from the woman. A blunt tip needle is inserted into the amniotic sac of the woman, attaching the blunt tip needle to a three-way stopcock. A Luer lock syringe is then connected to the three-way stopcock, connecting a first end of a length of sterile tubing with the three-way stopcock, and collecting the amniotic fluid through the blunt tip needle and sterile tubing into a collection container.

In this embodiment, the sterile collection container can include a pump with a suction device, typically a low suction device or a spring-loaded low suction device. The suction device is fluidly connected to an internal balloon. This embodiment further includes manually pumping up the internal balloon in the sterile collection container using the low suction device to allow a low-level suction and collection of the amniotic fluid.

In one embodiment, tools to obtain sterilely filtered human amniotic fluid from a woman, include a three-way stopcock, a sterile blunt tip needle aseptically attached to the three-way stopcock, a Luer lock syringe aseptically connected to the three-way stopcock, a sterile tubing aseptically connected to the three-way stopcock, a collection container or a collection container including a pump with suction device connected with the sterile tubing, a set of filters having pore size of about 5 m to about 10 m, a set of capsule or cartridge filters having the pore size of about 1 m, a set of capsule or cartridge filters having the pore size of about 0.45 m or 0.2 m, a set of sterile syringes or vials to store the sterile filtered amniotic fluid and, optionally, operating instructions on using the kit to obtain sterilely filtered human amniotic fluid. The filters having pore size of from about 5 m to about 10 m and the capsule or cartridge filters can be made from cellulose ester, glass fiber or nylon.

The sterile collection container may include a pump with a suction device, which may be a low suction device or spring-loaded low suction device. The suction device may be fluidly connected to an internal balloon. The internal balloon may be pumped up in the sterile collection container using the low suction device to allow a low-level suction and collection of the amniotic fluid. The sterile collection container may include a vent having a cap.

In one embodiment, utilizing the incision site immediately prior to performing the C-section and with ultrasound guidance to protect the fetus and mother provides a minimal or no risk environment for collection. Collection is achieved via low-level suction established within a collection container and/or via gravity. Typically, after high-speed centrifugation filtration with 5 to 10 m filters (low protein binding filter) is used to complete the removal of cells and large particles. Submicron filtration is then conducted with 1 m and 0.45 m or/and 0.2 m filters (low protein binding filter), two in a series connection, to remove gross contaminates. Under this condition, soluble growth factors will pass through this filter to achieve semi-sterile conditions. If under a strict aseptic operation condition, a 10⁻³ sterility assurance level is achieved. A 10⁻⁶ sterility assurance level can be achieved by submicron filtration with a 0.22 m filter (low protein binding filter) into sterile packaging to achieve a sterile product. The filtrate is monitored after each filtration step to determine which components were removed and then to determine which process to use to achieve the desirable product.

Cells, large particles and other undissolved particulates are removed from the human amniotic fluid by centrifuging or depth filtering the human amniotic fluid. In some embodiments, the human amniotic fluid is centrifuged at about 5,000 rpm to about 10,000 rpm for between about 30 minutes and about 60 minutes. In this embodiment filters of about 5 m to about 10 m are used for the first filtration. These are typically cellulose ester filters, glass fiber filters, nylon capsule filters or nylon cartridge filters. Filters with a pore size of 1.0 m are capsule filters or cartridge filters, typically formed of poly ether sulfone, poly vinylidene fluoride or cellulose acetate membranes. Filters with a pore size of 0.45 m or 0.2 m are capsule filters or cartridge filters, typically formed of poly ether sulfone membrane filters, poly vinylidene fluoride or cellulose acetate membranes.

One may use membrane filters including or made of hydrophilic polyethersulphone (PES) to filter protein solutions. Filter disks are used for small volumes, while a variety of cartridge sizes are used for larger volumes such as 1 liter and more. Hydrophobic membranes like PTFE (e.g., those designed for liquids devoid of proteins) should not be used. In one embodiment, the first step is centrifugation at 5000 to 8000 rpm for at least 30 minutes. Next, the supernatant is filtered with a prefilter to remove residual protein aggregates and precipitates in suspension (e.g., AP20). If one directly uses a 0.6/0.2 m filter, after prefiltration, one may experience slow filtration rates and the flow may stop too quickly. It may be desirable to make intermediate filtration steps using 1.2 m and 0.8 m membranes. Typically, a final filtration through 0.2 m is necessary to get the best sterility assurance level and produce a sterile amniotic fluid for injections. In some embodiments, the final filtrate is stored at about −20° C. to about −80° C. for long term storage.

The sterile amniotic fluid can be lyophilized to yield a lyophilizate (e.g., a freeze-dried product). The sterilely filtered amniotic fluid may be distributed in vials equipped with special rubber stoppers for sterile lyophilization. The lyophilization is carried out in a sterile environment. The rubber stoppers on the vials are then automatically pushed down in the freeze dryer to ensure robust closure. Finally, an aluminum cap is sealed on each vial to protect its sterile content.

The lyophilizate can be irradiated by e-beam irradiation or gamma ray irradiation to insure the sterility. The lyophilized amniotic fluid may be stored at +4° C. or room temperature for at least one year without observed degradation of its bioactive constituents. The sterile lyophilized amniotic fluid may be reconstituted by adding sterile water to the lyophilized powder.

2. Sources of Amniotic Fluid and d-MAPPS

Amniotic fluid formulations (e.g., d-MAPPS formulations) are prepared from sterile human amniotic fluid obtained from a pregnant woman and/or lyophilized amniotic fluid (e.g., reconstituted lyophilized amniotic fluid). In some embodiments, unprocessed amniotic fluid (“AF”) is first voluntarily obtained from patients who are undergoing amniocentesis, patients who are undergoing a Caesarean section, and/or patients undergoing normal delivery using a specially designed receptacle to collect the fluid after rupture of the relevant membranes. AF is then de-cellularized and otherwise treated as described herein to isolate d-MAPPS.

In other embodiments, amniotic fluid formulations (e.g., d-MAPPS formulations) are prepared from sterile amniotic fluid obtained from a pregnant humanized animal and/or animal-derived materials. For clinical application of d-MAPPS™ regenerative biologics platform technology (“d-MAPPS”), humanized animal-derived products from humanized animal feeder cells and/or a humanized animal can be grown and maintained in an undifferentiated state under the same culture conditions described above. In some embodiments, human amniotic fluid cells (“hAFCs”) may be used as feeder cells. Humanized animal cells/tissue is monitored for morphological changes compared with human placental tissue. Lyophilized amniotic fluid (e.g., reconstituted lyophilized amniotic fluid) from humanized animal cell lines may also be prepared.

De-cellularized human amniotic fluid (d-MAPPS) formulations can be stored for long periods of time, allowing for a variety of modes of application, including distribution and storage as aerosols, solutions, powders, etc. In some embodiments, the sterile d-MAPPS is refrigerated at about 1° C. to about 10° C. for long-term storage. In other embodiments, sterile d-MAPPS is refrigerated at 4° C. for up to 12 months or more. Preferably, long-term storage does not reduce the quantity or quality of the total soluble proteins and/or factors present in d-MAPPS. For some embodiments, the total soluble proteins retained after long-term storage in refrigerated conditions is about 10%, 20%, 30%, 40%, 50%, 60%, 70%, 80%, or 90% of the fresh d-MAPPS.

d-MAPPS formulations containing amniotic factors can be supplied as a clear one-part solution in a suitable container for storage at 4° C., or for storage at −20° C., or at −80° C. For example, liquid formulations in prefilled aliquots can be suitable for storage at 1-5° C., or for storage at −20° C., or at −80° C. The liquid formulation can be suitable for topical application in a nebulizer or an inhaler. In other embodiments, the fluid can be supplied as a kit that can be stored at 4° C., at −20° C., or at −80° C. until needed.

In some embodiments, d-MAPPS formulations use a final filtration through a 0.2 m filter. In some embodiments, this step is necessary to optimize sterile conditions without the requirement for irradiation (e.g., e-beam treatment). In some embodiments, d-MAPPS formulations have a 10⁻⁶ sterility assurance level without irradiation. In other embodiments, lyophilisate derived from amniotic fluid through lyophilization may also be irradiated by e-beam irradiation or gamma ray irradiation to fully sterilize the lyophilisate powder.

In embodiments, the sterilely filtered d-MAPPS™ regenerative biologics platform technology (“d-MAPPS”) comprises various growth factors. These growth factors exceed 300 in number and include human growth hormone, transforming growth factor beta 1, vascular endothelial growth factor, epidermal growth factor, transforming growth factor beta 3, growth differentiation factor 11, and/or combinations thereof.

B. Other Prophylactics and Therapeutic Agents

In some embodiments, sterile de-cellularized human amniotic fluid (e.g., d-MAPPS) is administered in combination with one or more additional therapeutic, diagnostic, and/or prophylactic agents to alleviate pain (e.g., pain associated with breast cancer), facilitate healing, and/or to reduce or inhibit scarring. In some embodiments, a d-MAPPS™ regenerative biologics platform technology (“d-MAPPS”) pharmaceutical composition administered comprises one or more additional compounds to prevent or treat cancers and tumors, and/or to relieve symptoms such as inflammation. Non-limiting examples include antimicrobial agents, analgesics, local anesthetics, anti-inflammatory agents, anti-oxidants, immunosuppressants, anti-allergenic agents, enzyme cofactors, essential nutrients and growth factors.

In some embodiments, d-MAPPS™ regenerative biologics platform technology administered to a subject for prevention or treatment of cancer and/or a tumor (e.g., a cancerous or non-cancerous tumor). In one example, d-MAPPS is administered adjacent to a site in need thereof via an effective amount of a sterile de-cellularized filtered non-heat-treated d-MAPPS formulation. In other embodiments, d-MAPPS is administered with a second cancer therapeutic (e.g., chemotherapy, humanized molecular antibody, etc.) to a subject for prevention or treatment of cancer and/or a tumor. Accordingly, d-MAPPS may be considered a targeted adjuvant therapy, serving to compliment traditional cancer therapeutic approaches (e.g. chemotherapy) while minimizing adverse side effects. Additional secondary therapeutic agents include but are not limited to antibiotics, cytokines, and growth factors such as fibroblast growth factor, hepatocyte growth factor, cell-cycle checkpoint inhibitors, platelet-derived growth factor, vascular endothelial cell growth factor, and insulin-like growth factor. In some embodiments, secondary therapeutic agents include hyaluronic acid or glycosaminoglycans.

In some embodiments, additional active agents may be administered with d-MAPPS, the active agents including small molecules, biomolecule, peptides, sugar, glycoproteins, polysaccharides, lipids, nucleic acids, and/or a combination thereof. Suitable small molecule active agents include organic and organometallic compounds. In some instances, the small molecule active agent has a molecular weight of less than about 2000 g/mol, more preferably less than about 1500 g/mol, most preferably less than about 1200 g/mol. The small molecule active agent can be a hydrophilic, hydrophobic, or amphiphilic compound. In some cases, one or more additional agents may be dispersed, dissolved or suspended in d-MAPPS, the d-MAPPS formulation, d-MAPPS carrier, and/or the d-MAPPS pharmaceutical composition.

Volume of administration of d-MAPPS is tissue-specific and dependent on the stage of the disease or disorder. Dosages can be readily determined by those of skill in the art. See Ansel, Howard C. et al. Pharmaceutical Dosage Forms and Drug Delivery Systems (6^{th} ed.) Williams and Wilkins, Malvern, Pa. (1995). Additionally, d-MAPPS may be administered in conjunction with exogenous stem cells, pluripotent cells, somatic cells, and/or combinations thereof. In other embodiments, one or more therapeutic, prophylactic or diagnostic agents is administered prior to, in conjunction with, or subsequent to treatment with d-MAPPS™ regenerative biologics platform technology (“d-MAPPS”). Notably, as used throughout this application, the term d-MAPPS is a patent element, which is used as an umbrella term to refer to numerous potential iterations of the platform technologies described throughout this application. Some elements of said platform technologies are protected by trade secret. Thus d-MAPPS is a patent element referring to a wide variety of different compositions and formulations, rather than a single closed formulation. In use, a medical professional would tailor d-MAPPS related medicaments or prophylactics for use with a specific indication and/or to the specific characteristics of a given patient. Relatedly, in some embodiments, therapeutic, prophylactic or diagnostic agents may be administered in a neutral form, or in the form of a pharmaceutically acceptable salt. In some cases, it may be desirable to prepare a formulation containing a salt of an agent due to one or more of the salt's advantageous physical properties, such as enhanced stability or a desirable solubility or dissolution profile.

In embodiments, pharmaceutically acceptable salts are prepared by reaction of the free acid or base forms of an active agent with a stoichiometric amount of the appropriate base or acid in water or in an organic solvent, or in a mixture of the two; generally, non-aqueous media like ether, ethyl acetate, ethanol, isopropanol, or acetonitrile are preferred. Pharmaceutically acceptable salts include salts of an active agent derived from inorganic acids, organic acids, alkali metal salts, and alkaline earth metal salts as well as salts formed by reaction of the drug with a suitable organic ligand (e.g., quaternary ammonium salts). Lists of suitable salts are found, for example, in Remington's Pharmaceutical Sciences, 20th ed., Lippincott Williams & Wilkins, Baltimore, Md., 2000, p. 704.

In some cases, the secondary agent administered with d-MAPPS comprises a diagnostic agent such as paramagnetic molecules, fluorescent compounds, magnetic molecules, and radionuclides, x-ray imaging agents, and/or contrast media.

In certain embodiments, a d-MAPPS pharmaceutical composition comprises one or more local anesthetics. Representative local anesthetics include tetracaine, lidocaine, amethocaine, proparacaine, lignocaine, and bupivacaine. In some cases, one or more additional agents, such as a hyaluronidase enzyme, is also added to the formulation to accelerate and improve dispersal of the local anesthetic.

In some embodiments, d-MAPPS™ regenerative biologics platform technology (“d-MAPPS”) is used in combination with one or more antimicrobial agents. An antimicrobial agent is a substance that inhibits the growth of microbes including bacteria, fungi, viruses, or parasites. Antimicrobial agents include antiviral agents, antibacterial agents, antiparasitic agents, and anti-fungal agents. Representative antiviral agents include ganciclovir and acyclovir. Representative antibiotic agents include aminoglycosides such as streptomycin, amikacin, gentamicin, and tobramycin, ansamycins such as geldanamycin and herbimycin, carbacephems, carbapenems, cephalosporins, glycopeptides such as vancomycin, teicoplanin, and telavancin, lincosamides, lipopeptides such as daptomycin, macrolides such as azithromycin, clarithromycin, dirithromycin, and erythromycin, monobactams, nitrofurans, penicillins, polypeptides such as bacitracin, colistin and polymyxin B, quinolones, sulfonamides, and tetracyclines.

Other exemplary antimicrobial agents include iodine, silver compounds, moxifloxacin, ciprofloxacin, levofloxacin, cefazolin, tigecycline, gentamycin, ceftazidime, ofloxacin, gatifloxacin, amphotericin, voriconazole, natamycin.

In some embodiments, d-MAPPS™ regenerative biologics platform technology (“d-MAPPS”) is administered in combination with one or more local anesthetics. A local anesthetic is a substance that causes reversible local anesthesia and has the effect of loss of the sensation of pain. Non-limiting examples of local anesthetics include ambucaine, amolanone, amylocaine, benoxinate, benzocaine, betoxycaine, biphenamine, bupivacaine, butacaine, butamben, butanilicaine, butethamine, butoxycaine, carticaine, chloroprocaine, cocaethylene, cocaine, cyclomethycaine, dibucaine, dimethysoquin, dimethocaine, diperodon, dycyclonine, ecgonidine, ecgonine, ethyl chloride, etidocaine, beta-eucaine, euprocin, fenalcomine, formocaine, hexylcaine, hydroxytetracaine, isobutyl p-aminobenzoate, leucinocaine mesylate, levoxadrol, lidocaine, mepivacaine, meprylcaine, metabutoxycaine, methyl chloride, myrtecaine, naepaine, octacaine, orthocaine, oxethazaine, parethoxycaine, phenacaine, phenol, piperocaine, piridocaine, polidocanol, pramoxine, prilocaine, procaine, propanocaine, proparacaine, propipocaine, propoxycaine, psuedococaine, pyrrocaine, ropivacaine, salicyl alcohol, tetracaine, tolycaine, trimecaine, zolamine, and any combination thereof. In other aspects of this embodiment, the sterile d-MAPPS composition includes an anesthetic agent in an amount of, e.g., about 0.1%, about 0.2%, about 0.3%, about 0.4%, about 0.5%, about 0.6%, about 0.7%, about 0.8% about 0.9%, about 1.0%, about 2.0%, about 3.0%, about 4.0%, about 5.0%, about 6.0%, about 7.0%, about 8.0%, about 9.0%, or about 10% by weight of the total composition.

In some embodiments, d-MAPPS is used in combination with one or more anti-inflammatory agents. Anti-inflammatory agents reduce inflammation and include steroidal and non-steroidal drugs. Suitable steroidal active agents include glucocorticoids, progestins, mineralocorticoids, and corticosteroids. Other exemplary anti-inflammatory agents include triamcinolone acetonide, fluocinolone acetonide, prednisolone, dexamethasone, loteprendol, fluorometholone, ibuprofen, aspirin, and naproxen. Exemplary immune-modulating drugs include cyclosporine, tacrolimus and rapamycin. Exemplary non-steroidal anti-inflammatory drug include ketorolac, nepafenac, and diclofenac. In some embodiments, anti-inflammatory agents are anti-inflammatory cytokines. Exemplary cytokines are IL-10, IL-17, TNF-α, TGF-β and IL-35.

In some embodiments, d-MAPPS is administered in combination with one or more growth factors. Growth factors refer to protein and/or glycoproteins capable of stimulating cellular growth, proliferation, and/or cellular differentiation. Non-limiting examples of growth factors include transforming growth factor beta (TGF-β), transforming growth factor alpha (TGF-α), granulocyte-colony stimulating factor (GCSF), granulocyte-macrophage colony stimulating factor (GM-CSF), nerve growth factor (NGF), neurotrophins, platelet-derived growth factor (PDGF), erythropoietin (EPO), thrombopoietin (TPO), myostatin (GDF8), growth differentiation factor-9 (GDF9), acidic fibroblast growth factor (aFGF or FGF-1), basic fibroblast growth factor (bFGF or FGF-2), epidermal growth factor (EGF), vascular endothelial growth factor (VEGF) and hepatocyte growth factor (HGF).

In some embodiments, the d-MAPPS composition is administered in combination with one or more enzyme cofactors, and/or one or more essential nutrients. Exemplary cofactors include vitamin C, biotin, vitamin E, and vitamin K. Exemplary essential nutrients are amino acids, fatty acids, etc.

In some embodiments, the d-MAPPS composition further comprises at least one eukaryotic cell type not present in the original amniotic fluid. Some exemplary eukaryotic cell types include stem cells, mesenchymal stem cells, immune cells such as T lymphocytes, B lymphocytes, natural killer cells, macrophages, dendritic cells, or combinations thereof. In some embodiments, the cells used are cells that dampen inflammation response such as regulatory T cells. In some embodiments, exosomes are generated ex vivo from mesenchymal stem cells (e.g., amniotic fluid mesenchymal stem cells).

C. d-MAPPS Formulations

In some embodiments, the sterile d-MAPPS formulations are packaged, for example, into sterile dosage units which can be stored and distributed for use by attending physicians. These lyophilized or fluid formulations can be in the form of sterile packaged syringes for injection, dropper bottles, tubes or vials of solution. The dosages for the injectables typically will be 0.1 cc, 0.25 cc, 0.5 cc, 1.0 cc, 2 cc, 5 cc, 10 cc, and 20 cc. The injectables can be administered at the site of injury. In one embodiment, the formulation is sprayed onto, soaked into, or powder dispersed onto the tumor site or cancer lesion. The efficacy of this process is determined by physician evaluations, patient self-evaluations, and Quality of life evaluations.

In the some embodiments, sterile d-MAPPS formulations can be administered in concentrated form, diluted with sterile water or buffer, or formulated as a solution or suspension. d-MAPPS formulations may be administered with additional therapeutic, prophylactic or diagnostic agents, either in solution or suspension, or as particles (nanoparticles, liposomes, microparticles) or directly at tumor sites.

Representative excipients include solvents, diluents, pH modifying agents, preservatives, antioxidants, suspending agents, wetting agents, viscosity modifiers, tonicity agents, stabilizing agents, and combinations thereof. Suitable pharmaceutically acceptable excipients are preferably selected from materials which are generally recognized as safe (GRAS), and may be administered to an individual without causing undesirable biological side effects or unwanted interactions.

1. d-MAPPS Solutions and Suspensions

In some embodiments, d-MAPPS is formulated into a solution or suspension. In some embodiments, solutions may comprise sterile filtered amniotic fluid, concentrated or diluted with water, buffered saline, or an equivalent, or emulsified with lipid or oil. Emulsions are generally dispersions of oily droplets in an aqueous phase. There should be no evidence of breaking or coalescence. Suspensions contain solid particles dispersed in a liquid vehicle; they must be homogeneous when shaken gently and remain sufficiently dispersed to enable the correct dose to be removed from the container. A sediment may occur, but this should disperse readily when the container is shaken, and the size of the dispersed particles should be controlled. The active ingredient and any other suspended material must be reduced to a particle size small enough to prevent irritation and damage to the site of administration. Suspensions may comprise suitable additives, such as antimicrobial agents, antioxidants, and stabilizing agents. In some embodiments, when the solution is dispensed in a multidose container that is to be used over a period of time longer than 24 hours, a preservative must be added to ensure microbiologic safety over the period of use.

In some embodiments, the pH of d-MAPPS solution or suspension is physiological, for example, pH 7.4. In some embodiments, the pH is optimized for both stability of the active pharmaceutical ingredient and physiological tolerance. If a buffer system is used, it must not cause precipitation or deterioration of the active ingredient. The normal useful range is 6.5 to 8.5, although lower pH may be used. Buffers and/or pH adjusting agents or vehicles can be added to adjust and stabilize the pH at a desired level. These buffers are included to minimize any change in pH during the storage life of d-MAPPS™ regenerative biologics platform technology (“d-MAPPS”). Changes in pH can affect the solubility and stability of drugs; consequently, it is important to minimize fluctuations in pH. The buffer system should be sufficient to maintain the pH throughout the expected shelf-life of the product. Low concentrations of buffer salts are used to prepare buffers of low buffer capacity.

Aqueous solution preparation is optimized/supplemented for isotonicity, pH, antimicrobial agents, antioxidants, and viscosity-increasing agents. Solutions are considered isotonic when the tonicity is equal to that of a 0.9% solution of sodium chloride. Tissue can usually tolerate solutions equivalent to 0.5-2% of sodium chloride. Solutions that are isotonic are preferred. An amount equivalent to 0.9% NaCl is used in the preferred embodiment. In some embodiments, hypertonic solutions are prepared to facilitate solubility of other agents coadministered with d-MAPPS. A widely used buffer solution is Sorensen's modified phosphate buffer. Sorensen's modified phosphate buffer is used to modulate pH values between the range of 6.5-8.0. This buffer comprises two stock solutions, one acidic containing NaH₂PO₄, and one basic containing Na₂HPO₄. Other suitable buffers known in the art include acetate, borate, carbonate, citrate, and phosphate buffers.

In some instances, the d-MAPPS formulation is distributed or packaged in liquid form. Alternatively, formulations can be packed as a solid, obtained, for example by lyophilization of a suitable liquid formulation. The solid can be reconstituted with an appropriate carrier or diluent prior to administration. Solutions, suspensions, or emulsions for administration to a subject may also contain one or more tonicity agents to adjust the isotonic range of the formulation. Suitable tonicity agents are well known in the art and some examples include glycerin, mannitol, sorbitol, sodium chloride, and other electrolytes.

Solutions, suspensions, aerosols, sprays or emulsions may also contain one or more preservatives to prevent bacterial contamination of the ophthalmic preparations. Suitable preservatives are known in the art, and include polyhexamethylenebiguanidine (PHMB), benzalkonium chloride (BAK), stabilized oxychloro complexes (otherwise known as PURITE®), phenylmercuric acetate, chlorobutanol, sorbic acid, chlorhexidine, benzyl alcohol, parabens, thimerosal, and mixtures thereof.

Solutions, suspensions, or emulsions may also contain one or more excipients known in the art, such as dispersing agents, wetting agents, and suspending agents.

D. d-MAPPS Kits

In some embodiments, d-MAPPS pharmaceutical compositions are provided in a kit. Formulations are prepared using a pharmaceutically acceptable “carrier” composed of materials that are considered safe and effective and may be administered to an individual without causing undesirable side effects. These lyophilized or fluid formulations can be in the form of sterile packaged syringes for injection, or tubes or jars of solution. The dosages for the injectables will be 0.1 cc, 0.25 cc, 0.5 cc, 1.0 cc, 2 cc, 5 cc, 10 cc, and 20 cc. Typically sterile d-MAPPS kits comprise at least lyophilized d-MAPPS plus a liquid to rehydrate the dry components. The kit may also include various elements facilitating administration of prophylactics or treatments of cancer, tumors, and other disorders, such as syringes and an applicator such as a needle.

### III. Methods of d-MAPPS Administration

Methods of using sterile, de-cellularized human amniotic fluid compositions (e.g. d-MAPPS) for cancer therapeutic, diagnostic, and prophylactic applications, especially with respect to cancers, tumors, and other related disorders are further disclosed herein.

In some embodiments, d-MAPPS pharmaceutical compositions are administered to any mammalian subject (e.g., terrestrial mammal, aquatic mammal, and the like). The decellularized amniotic fluid (e.g., d-MAPPS) is administered in a dosing regimen and for a period of time effective to prevent formation of tumors and/or to promote healing, repair and/or regeneration of soft-tissue tumors.

In some embodiments, d-MAPPS compositions experience limited perfusion and therefore may be retained at the site of application/injection for an extended period of time. In one embodiment, after administration, the d-MAPPS composition remains at the site of application for at least 6 hours, at least 12 hours, at least 1 day, at least 2 days, at least 3 days, at least one week, at least 2 weeks, at least 3 weeks, at least 4 weeks, at least 5 weeks, at least 6 weeks, at least 7 weeks, at least 2 months, at least 3 months, at least 6 months, at least 9 months, at least 1 year or more.

A. Cancers, Tumors and Other Disorders to be Prevented and Treated

Methods of using the d-MAPPS and d-MAPPS formulations to prevent or treat cancer (e.g., blood cancers, and other cancers described herein), tumors and other disorders are described herein. In some embodiments, the methods and compositions are effective in preventing and/or treating cancers (e.g., breast cancer) and other non-cancerous tumors. In further embodiments, the formulation is administered in an amount effective to restore tissue impacted by cancer and/or tumor growth to about 10%, 20%, 30%, 40%, 50%, 60%, 70%, 80%, 90%, 100%, 150%, 200%, 250%, 300%, or more than 300% of the damage present at the time of treatment, as measured by endogenous tissue regrowth.

In one embodiment, the d-MAPPS formulation is administered by injection near the site of injury or tumor infarction. In another embodiment, the d-MAPPS formulation is sprayed onto, soaked into, or powder dispersed onto the site of tumor growth.

The compositions and methods of use thereof are suitable for managing or treating any cancer or tumor, in addition to other associated diseases and disorders. For example, d-MAPPS administration may prevent or treat cancer in a patient with a degenerative disease, contributing to the reduction of symptoms of both the cancer and degenerative disease.

1. Cancer and Tumors

Disclosed herein are methods of preventing or treating cancer (such as breast cancer, blood cancers, pancreatic adenocarcinoma, or colorectal adenocarcinoma) via the administration of d-MAPPS™ regenerative biologics platform technology (“d-MAPPS”). In some embodiments, d-MAPPS is administered to a cancer patient or potential cancer patient in combination with radiation therapy and/or chemotherapy. In some embodiments, the methods include administering to the subject a composition including a d-MAPPS formulation and a pharmaceutically acceptable carrier. In other examples, the methods include administering to the subject a pharmaceutical composition including an expression vector expressing costimulatory molecules, the d-MAPPS formulation, and a pharmaceutically acceptable carrier.

In some embodiments, methods of preventing tumor growth (e.g., breast cancer tumor growth) or treating a subject with a tumor include measuring a tumor sample or tumor volume from a subject, determining an appropriate dosage of d-MAPPS, and treating the subject. In some embodiments, treating the subject may include administering to the subject an effective amount of ionizing radiation in combination with an effective amount of d-MAPPS. In another embodiment, d-MAPPS is administered in combination with one or more adjuvants, antigens, vaccines, allergens, antibiotics, gene therapy vectors, vaccines, kinase inhibitors, co-stimulatory molecules, TLR agonists, or TLR antagonists. In some embodiments, d-MAPPS is administered in combination with a second anti-cancer therapeutic agent (e.g., a chemotherapeutic nucleic acid, an immunostimulatory protein, an inflammatory molecule, and/or a immunostimulatory molecule). In other embodiments, the d-MAPPS is administered systemically or at specific tumor locations in the subject.

In some embodiments, the invention pertains to a method for treating a subject with cancer by enhancing or inducing response of cancer-associated endogenous immune cells in the subject. In some embodiments, enhancing or inducing response of cancer-associated endogenous immune cells may include administering to the subject an effective amount d-MAPPS as a prophylactic (e.g., an amount effective at preventing the appearance of tumors). In some embodiments, enhancing or inducing response of cancer-associated endogenous immune cells may include administering to the subject an effective amount d-MAPPS to treat a subject with cancer or a tumor. In other embodiments, enhancing or inducing response of cancer-associated endogenous immune cells may include administering to the subject an effective amount of ionizing radiation, then administering to the subject an effective amount of d-MAPPS, thereby enhancing or inducing the response of cancer-associated endogenous immune cells in the subject. In particular embodiments, said cancer-associated endogenous immune cells may include dendritic cells, macrophages, T cells, natural killer cells, and the like.

In other embodiments, d-MAPPS is administered in combination with one or more checkpoint inhibitors. In particular examples, checkpoint inhibitors may target one or more of PD-1, PD-L1 (B7-H1), OX40/OX-40L, CTLA-4, and LAG3. Other checkpoint targets may include, but are not limited to: checkpoint target immune checkpoint targets, such as PD-2, PD-L2, IDO 1 and 2, CTNNB1 (β-catenin), SIRPα, VISTA, RNASE H2, DNase II, CLEVER-1/Stabilin-1, LIGHT, HVEM, TIM3, TIGIT, Galectin-9, KIR, GITR, TIM1, TIM4, CEACAM1, CD27, CD40/CD40L, CD48, CD70, CD80, CD86, CD112, CD137 (4-1BB), CD155, CD160, CD200, CD226, CD244 (2B4), CD272 (BTLA), B7-H2, B7-H3, B7-H4, B7-H6, ICOS, A2aR, A2bR, HHLA2, ILT-2, ILT-4, gp49B, PIR-B, HLA-G, and ILT-2/4. Other targets include MDR1, Arginasel, iNOs, IL-10, TGF-β, pGE2, STAT3, VEGF, KSP, HER2, Ras, EZH2, NIPP1, PP1, TAK1 and PLK1.

Other examples of cancers include hematological malignancies including leukemias, including acute leukemias (such as 11q23-positive acute leukemia, acute lymphocytic leukemia (ALL), T-cell ALL, acute myelocytic leukemia, acute myelogenous leukemia (AML), and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), lymphoblastic leukemia, polycythemia vera, lymphoma, diffuse large B cell lymphoma, Burkitt lymphoma, T cell lymphoma, follicular lymphoma, mantle cell lymphoma, Hodgkin disease, non-Hodgkin lymphoma, multiple myeloma, Waldenstrom macroglobulinemia, heavy chain disease, myelodysplastic syndrome, hairy cell leukemia, and myelodysplasia. The compositions and methods provided herein are also used to treat NSCLC (non-small cell lung cancer), pediatric malignancies, cervical and other tumors caused or promoted by human papilloma virus (HPV), melanoma, Barrett's esophagus (pre-malignant syndrome), adrenal and skin cancers and auto immune, neoplastic cutaneous diseases.

In other embodiments, the methods and d-MAPPS pharmaceutical provided herein are used to prevent or treat multiple cancers. In some embodiments, d-MAPPS is be administered to a subject with both cancer and another disorder such as systemic inflammation or a neurodegenerative disease. A cell, tissue, or target may be a cancer cell, a cancerous tissue, harbor cancerous tissue, or be a subject or patient diagnosed or at risk of developing a disease or condition. In certain aspects, a cell may be an epithelial, an endothelial, a mesothelial, a glial, a stromal, or a mucosal cell. The cancer cell population can include, but is not limited to a brain, a neuronal, a blood, an endometrial, a meninges, an esophageal, a lung, a cardiovascular, a liver, a lymphoid, a breast, a bone, a connective tissue, a fat, a retinal, a thyroid, a glandular, an adrenal, a pancreatic, a stomach, an intestinal, a kidney, a bladder, a colon, a prostate, a uterine, an ovarian, a cervical, a testicular, a splenic, a skin, a smooth muscle, a cardiac muscle, or a striated muscle cell. In still a further aspect cancer includes, but is not limited to astrocytoma, acute myeloid leukemia, anaplastic large cell lymphoma, acute lymphoblastic leukemia, angiosarcoma, B-cell lymphoma, Burkitt's lymphoma, breast carcinoma, bladder carcinoma, carcinoma of the head and neck, cervical carcinoma, chronic lymphoblastic leukemia, chronic myeloid leukemia, colorectal carcinoma, endometrial carcinoma, esophageal squamous cell carcinoma, Ewing's sarcoma, fibrosarcoma, glioma, glioblastoma, gastrinoma, gastric carcinoma, hepatoblastoma, hepatocellular carcinoma, Kaposi's sarcoma, Hodgkin lymphoma, laryngeal squamous cell carcinoma, larynx carcinoma, leukemia, leiomyosarcoma, lipoma, liposarcoma, melanoma, mantle cell lymphoma, medulloblastoma, mesothelioma, myxofibrosarcoma, myeloid leukemia, mucosa-associated lymphoid tissue B cell lymphoma, multiple myeloma, high-risk myelodysplastic syndrome, nasopharyngeal carcinoma, neuroblastoma, neurofibroma, high-grade non-Hodgkin lymphoma, non-Hodgkin lymphoma, lung carcinoma, non-small cell lung carcinoma, ovarian carcinoma, oesophageal carcinoma, osteosarcoma, pancreatic carcinoma, pheochromocytoma, prostate carcinoma, renal cell carcinoma, retinoblastoma, rhabdomyosarcoma, salivary gland tumor, Schwanomma, small cell lung cancer, squamous cell carcinoma of the head and neck, testicular tumor, thyroid carcinoma, urothelial carcinoma, and Wilm's tumor.

Examples of solid tumors, include sarcomas (such as fibrosarcoma, myxosarcoma, liposarcoma, chondrosarcoma, osteogenic sarcoma, and other sarcomas), synovioma, mesothelioma, Ewing sarcoma, leiomyosarcoma, rhabdomyosarcoma, colon cancer, colorectal cancer, peritoneal cancer, esophageal cancer, pancreatic cancer, breast cancer (including basal breast carcinoma, ductal carcinoma and lobular breast carcinoma), lung cancer, ovarian cancer, prostate cancer, liver cancer (including hepatocellular carcinoma), gastric cancer, squamous cell carcinoma (including head and neck squamous cell carcinoma), basal cell carcinoma, adenocarcinoma, sweat gland carcinoma, medullary thyroid carcinoma, papillary thyroid carcinoma, pheochromocytoma, sebaceous gland carcinoma, papillary carcinoma, papillary adenocarcinoma, medullary carcinoma, bronchogenic carcinoma, hepatoma, bile duct carcinoma, choriocarcinoma, Wilms tumor, cervical cancer, fallopian tube cancer, testicular tumor, seminoma, bladder cancer (such as renal cell cancer), melanoma, and CNS tumors (such as a glioma, glioblastoma, astrocytoma, medulloblastoma, craniopharyrgioma, ependymoma, pinealoma, hemangioblastoma, acoustic neuroma, oligodendroglioma, meningioma, neuroblastoma and retinoblastoma). Solid tumors also include tumor metastases (for example, metastases to the lung, liver, brain, or bone).

In some embodiments, tumors comprise non-cancerous tumors such as benign soft tissue tumors. Benign soft tissue tumors may include lipoma, angiolipoma, fibroma, benign fibrous histiocytoma, neurilemmona, hemangioma, giant cell tumor of tendon sheath, myxoma, and the like. In other embodiments, d-MAPPS may be administered as a prophylactic or treatment for other non-cancerous soft tissue tumors including fat tissue tumors (e.g., lipoblastoma, hibernoma), fibrous tissue tumors (e.g., elastofibroma, superficial fibromatosis, desmoid-type fibromatosis, and deep benign fibrous histiocytoma), muscle tissue tumors (e.g., leiomyomas, and rhabdomyoma), blood and lymph vessel tumors (e.g., hemangioma, glomus tumors, and lymphangioma), and nerve tissue tumors (e.g., neurofibroma and schwannoma).

In embodiments, the methods described herein may include the step of identifying and selecting a subject in need of treatment, or a subject who would benefit from administration of d-MAPPS. In some embodiments, the subject to be treated is a mammal (e.g., a human, domestic animal, livestock, aquatic mammal, and the like).

A variety of pharmaceutically acceptable carriers can be used with the d-MAPPS compositions provided herein. For example, in some embodiments, buffered saline and the like may be used with the d-MAPPS compositions provided herein. Optionally, these solutions may be sterilized prior to use. Other suitable carriers can include, but are not limited to, water, phosphate buffered saline solutions, phosphate buffered saline containing Polysorbate 80, emulsions such as oil/water emulsion and various type of wetting agents, salt solutions, alcohols, gum arabic, vegetable oils, benzyl alcohols, aqueous vehicles, water-miscible vehicles, nonaqueous vehicles (e.g., corn oil, cottonseed oil, peanut oil, sesame oil, ethyl oleate, isopropyl myristate, and benzyl benzoate), etc. Carriers also include, e.g., milk, sugar, certain types of clay, silica, gelatin, stearic acid or salts thereof, magnesium, magnesium stearate and other forms or salts of magnesium, or calcium stearate, talc, vegetable fats or oils, gums, glycols, propylene glycol, buffers, antimicrobial agents, preservatives, flavor, fragrance and color additives, gelatin, carbohydrates such as lactose, amylose or starch, talc, silicic acid, viscous paraffin, perfume oil, fatty acid monoglycerides and diglycerides, pentaerythritol fatty acid esters, hydroxy methylcellulose and the like. Other additives include, e.g., antioxidants and preservatives, coloring, flavoring and diluting agents, emulsifying and suspending agents, such as acacia, agar, alginic acid, sodium alginate, bentonite, carbomer, carrageenan, carboxymethylcellulose, cellulose, cholesterol, fatty acids, triglycerides and esters of fatty acids, fatty alcohols, gelatin, hydroxyethyl cellulose, hydroxypropyl cellulose, hydroxypropyl methylcellulose, methylcellulose, octoxynol 9, oleyl alcohol, povidone, propylene glycol monostearate, sodium lauryl sulfate, sorbitan esters, stearyl alcohol, tragacanth, xanthan gum, and derivatives thereof, solvents, transdermal enhancers (ethanol, propylene glycol, water, sodium oleate, leucinic acid, oleic acid, capric acid, sodium caprate, capric/caprylic triglyceride, silica, lauric acid, sodium laurate, neodecanoic acid, dodecyl-amine, cetryl lactate, myristyl lactate, lauryl lactate, methyl laurate, phenyl ethanol, hexa-methylene lauramide, urea and derivatives, dodecyl N, N-dimethylamino acetate, hydroxyethyl lactamide, phyophatidylcholine, sefsol-318 (a medium chain glyceride), isopropyl myristate, isopropyl palmitate, palmitic acid, several surfactants, including poly-oxyethylene (10) lauryl ether (Brij 361 R), diethyleneglycol lauryl ether (PEG-2-L), laurocapram (Azone; 1,1-dodecylazacycloheptan-2-one), acetonitrile, 1-decanol, 2-pyrrolidone, N-methylpyrrolidone, N-ethyl-1-pyrrolidone, 1-Methyl-2-pyrrolidone, 1-lauryl-2-pyrrolidone, sucrose monooleate, dimethylsulfoxide (DMSO) about 80% concentration required, decylmethylsulfoxide (n) enhances primarily polar or ionic molecules (soluble in ethanol), acetone, polyethylene glycol 100-400 MW, dimethylacetamide, dimethylformamide, dimethylisosorbide, sodium bicarbonate, various N7-16-alkanes, mentane, menthone, menthol, terpinene, D-terpinene, dipen-tene, N-nonalool and limonene, skin penetration enhancers (e.g., lecithin), and miscellaneous ingredients such as microcrystalline cellulose, citric acid, dextrin, dextrose, liquid glucose, lactic acid, lactose, magnesium chloride, potassium metaphosphate, starch, and the like.

In some examples, d-MAPPS™ regenerative biologics platform technology (“d-MAPPS”) compositions include pharmaceutically acceptable auxiliary substances such as pH adjusting and buffering agents, toxicity adjusting agents, and preservatives, for example, sodium acetate, sodium chloride, potassium chloride, calcium chloride, sodium lactate and the like. The concentration of these formulations can vary depending on individual differences in age, weight, tumor size, extent of metastasis, and condition of the patient (subject).

Methods related to the d-MAPPS compositions and formulations and their use are provided. The d-MAPPS compositions and formulations may be prepared as pharmaceutical compositions (e.g., amniotic fluid compositions or formulations in combination with a pharmaceutically acceptable buffer, carrier, diluent or excipient) for use in the methods. For example, disclosed are methods of administration of the compositions, methods of inducing or increasing the expansion and/or function of CD4+ T regulatory cells ex vivo or in vivo. Also disclosed are methods of inducing or increasing a population of DC or NK cells, for example, in a subject in need thereof. The methods of treatment can include administering to a subject (e.g., a human patient) an effective amount of a d-MAPPS pharmaceutical composition including the de-cellularized amniotic fluid to one or more cancerous or tumorigenic tissues in the subject.

In some embodiments, d-MAPPS administration to a subject results in an increase in the proliferation or the number of endogenous immune cells (e.g. anti-inflammatory cells). Generally, this increase is observed within days, weeks, or months after the initial treatment, with an observed increase up to 10%, 20%, 30%, 40%, 50%, 60%, 70%, 80%, 90%, 100%, 200%, 300%, 400%, 500% or more than 500%.

2. d-MAPPS-Associated MSCs as Cancer Therapeutic Agents

In some embodiments, d-MAPPS pharmaceutical compositions (“d-MAPPS”) comprise one or more exosomes (“Exos”) generated ex vivo from mesenchymal stem cells. In the preferred embodiment, said mesenchymal stem cells include d-MAPPS-associated MSCs (“dMSCs”), wherein dMSCs comprise amniotic fluid mesenchymal stem cells (“AF MSCs”), placental tissue-derived mesenchymal stem cells (“PL-MSC”), cancer-associated MSCs (“CA MSCs”), and/or exogenously administered MSCs.

In some embodiments, dMSCs that do not express MHC class II molecules are transplanted in MHC-mismatched cancer patients. In other embodiments, dMSCs express various chemokine receptors and after intravenous injection migrate to the tumor tissue to participate in the anti-tumor immune response. Because of their low immunogenicity and tumor-homing properties, also disclosed are the use of dMSCs as delivery vehicles for the delivery of bi-specific T cell-engaging antibodies, protein engagers that simultaneously bind to the tumor antigen and appropriate ligand on T lymphocytes, enabling the specific T cell-mediated elimination of tumor cells.

In some embodiments, glypican 3 (GPC3) is disclosed to regulate the proliferation of hepatocellular carcinoma cells and therefore serves as a useful target. In one example, GPC3-specific single-chain variable fragment (scFv) and CD3-specific scFv (MSC^{GPC3-CD3}) are expressed to direct GPC3-specific CD4+T helper cells and CD8+ CTLs towards the GPC3-expressing hepatocellular carcinoma cells. In some embodiments, co-culture of GPC3+ tumor cells, MSCs^{GPC3-CD3} and T lymphocytes leads to the increased production of IFN-γ in GPC3-specific CD4+T cells and the enhanced activation and expansion of GPC3-specific CTLs, which result in the efficient CTL-dependent killing of GPC3-expressing malignant cells. In other embodiments, increased activation of GPC3-specific T cells and significantly reduced hepatocellular carcinoma growth are observed in MSCs^{GPC3-CD3}-treated tumor-bearing mice, demonstrating the therapeutic potential of MSCs^{GPC3-CD3} in the immunotherapy of hepatocellular carcinoma.

In some embodiments, low doses of ultraviolet radiation and X-ray irradiation generate an anti-tumorigenic MSC1 phenotype in dMSCs, and therefore may be used for MSC priming. Irradiated BM-MSC1 secretes a large amount of TNF-α and IFN-γ, which: (i) inhibit the proliferation of tumor cells by deregulating Wnt and TGF-β/Smad signaling and (ii) induce the apoptosis of tumor cells by blocking their cell cycle in the G1 phase. Additionally, MSC-sourced TNF-α induces the necrosis of tumor cells and enhances the expression of E and P selectins on tumor endothelial cells, enabling a massive influx of immune cells. dMSC-sourced IFN-γ induces the generation of the anti-tumorigenic (M1) phenotype in TAMs and enhances the cytotoxicity of tumor-infiltrated CTLs and NK cells. Upon activation by dMSC-derived IFN-γ, CD8+ CTLs and NK cells up-regulate the expression of FASL and TRAIL and increase the release of perforin and granzymes that induce the apoptosis of tumor cells. IFN-γ-primed M1 macrophages either phagocyte apoptotic tumor cells or secrete ROS, NO and TNF-α, which have direct cytotoxic effects on malignant cells.

In some embodiments, dMSC-derived extracellular vesicles (dMSC-EVs) are administered to a subject to prevent or treat cancer, tumors, and associated diseases. In embodiments, dMSC-EVs comprise dMSC-sourced anti-tumorigenic microRNAs (miRNAs), which represent a novel therapeutic approach in the MSC-based immunotherapy of tumors. In other embodiments, alternate nucleic acid systems (e.g., trans-cleaving ribozyme, siRNA, snRNA, and the like) are contemplated to serve a similar role as the dMSC-sourced anti-tumorigenic microRNAs (miRNAs). Due to the lipid envelope, dMSC-EVs easily by-pass all biological barriers and deliver their cargo directly into the target cells. Accordingly, in some embodiments, dMSC-EVs are adapted to deliver dMSC-sourced anti-tumorigenic miRNAs directly into tumor cells, altering their viability, proliferation rate and invasive characteristics. In one embodiment, human BM-MSC-EV-sourced miRNA-16-5p and miRNA-3940-5p from human umbilical cord-derived dMSC-EVs (UC-MSC-EVs) are administered to inhibit the migratory properties and metastatic potential of tumor cells by down-regulating the expression of Integrin Subunit Alpha (ITGA)2 and ITGA6 on their membranes. In another embodiment, human BM-MSC-EV-delivered miRNA-4461 suppresses the proliferation and invasive properties of tumor cells by reducing the expression of COPB2, which is essential for Golgi budding and vesicular trafficking. In other embodiments, human adipose tissue-derived MSC-EVs (AT-MSC-EVs) carrying miRNA-15a inhibit the immune escape of tumor cells by regulating the expression of homeobox C4 (HOXC4), which binds to the promoter sequence of PDL1, controlling its synthesis and membrane expression. Additionally, human AT-MSC-EV-derived miRNA-15a induces the apoptosis of tumor cells by inhibiting the activity of histone lysine demethylase 4B (KDM4B), which epigenetically regulates chromatin structure. In yet another embodiment, human BM-MSC-EV-delivered miRNA-100 down-regulates the production of VEGF in cancer cells, preventing the generation of new blood vessels in growing tumors.

In another embodiment, dMSCs are used to target delivery agents of anti-cancer drugs. Reduced numbers of lung metastases were noticed in melanoma-bearing animals that received dMSCs loaded with the anti-cancer drug paclitaxel (PTX). In other embodiments, enhanced anti-tumor properties of PTX-loaded nano- and glyco-engineered dMSCs are observed against murine and ovarian cancer. As vehicles, dMSCs have many advantages as compared to other drug administration agents. For example, anti-neoplastic drug-loaded dMSCs release chemotherapeutics directly in the site of primary and metastatic tumors without affecting neighboring tissues. Accordingly, reduced side effects, an increased half-life and better anti-tumor effects are achieved in experimental animals that receive anti-cancer drug-loaded dMSCs compared to chemotherapeutic-treated tumor-bearing animals.

In some embodiments, CA-MSCs are provided to suppress dendritic cell-dependent activation of T cells in a subject with cancer, a tumor, or the like. For example, CA-MSC-derived IL-10 may inhibit the DC-induced proliferation of T cells by blocking the ability of dendritic cells (“DCs”) to provide cysteine to cognate T lymphocytes. In another embodiment, CA-MSC-derived IL-10 may induce the phosphorylation of STAT-3 in DCs. In embodiments, phosphorylated STAT-3 enters the nucleus and represses the interferon gamma-activated sequence (GAS) which serves as a cystathionase promoter sequence. As a consequence, DC-derived cysteine export to T cells is suppressed, resulting in reduced T cell proliferation and activation. In embodiments, lack of cysteine significantly attenuates the production of IFN-γ in T cells and therefore alleviates their capacity to activate macrophages in an IFN-γ-dependent manner. Thus, in embodiments, d-MAPPS-associated MSCs may serve to abrogate the immune system's defense against cancerous lesions.

In some embodiments, d-MAPPS and/or d-MAPPS-associated MSCs are administered to a subject in a manner that facilitates regulation of tumor progression. In some embodiments, d-MAPPS-associated MSCs are treated with condition medium derived from TNFα, IL-1β, and iNOS-expressing M1 macrophages, thereby increasing the expression of toll-like receptor 3 (TLR-3) on dMSCs. In embodiments, TLR-3 signaling increases expression of inducible nitric oxide synthase (iNOS), CCL2, IL-6 and cyclooxygenase 2 (COX-2). In some embodiments, nucleic acid therapeutics (e.g., miRNA, snRNA, siRNA, and the like) are used to inhibit iNOS activity and NO production, thereby reducing immunosuppressive responses of subjects during cancer treatment. In some embodiments, dMSCs display an increased capacity for the production of IL-6 and COX-2, which induce the generation of the anti-inflammatory phenotypes. In some embodiments, d-MAPPS is co-administered with program death ligand 1(PDL1) inhibitor, which enhances the cytotoxic T lymphocytes CTL-dependent elimination of tumor cells.

In some embodiments, dMSCs are treated such that they adopt the phenotype and function under the influence of the biological factors to which they are exposed. For example, dMSCs may obtain pro-inflammatory (MSC1) and anti-inflammatory (MSC2) phenotypes depending on the local tissue concentration of inflammatory cytokines, TNF-α and IFN-γ. In some embodiments, dMSCs are engrafted in the tissue with a low level of TNF-α and IFN-γ, thereby obtaining a pro-inflammatory MSC1 phenotype and secreting a large number of inflammatory factors (reactive oxygen species (ROS) IL-1β, interferon alpha and beta (IFN-α, IFN-β), TNF-α and IFN-γ), which enhance the phagocytic properties of neutrophils and macrophages and the cytotoxicity of CTLs and NK cells. In another embodiment, when dMSCs are exposed to high levels of inflammatory cytokines (TNF-α and IFN-γ), they acquire an immunosuppressive MSC2 phenotype characterized by the increased production of anti-inflammatory factors (TGF-β, IL-10, PGE2, NO, IDO, IL-1Ra) that suppress the effector function of inflammatory endogenous immune cells and attenuate on-going inflammation. In another embodiment, TNF-α and IFN-γ-primed MSC2 express and secrete PDL1 and PDL2, which suppress the proliferation of TNF-α and IFN-γ-producing T cells and promote the generation and expansion of immunosuppressive Tregs.

In some embodiments, the dMSCs of d-MAPPS are exogenously administered to a subject as a melanoma cancer preventative treatment. In embodiments, dMSCs transplanted during the initial phase of melanoma growth (e.g., importantly, not during the progressive stage of growth) exert a tumor-suppressive effect. In other embodiments, dMSCs are intravenously injected 24 h after melanoma induction in order to enhance the cytotoxicity of CD8+ CTLs and NK cells, increase the production of anti-tumorigenic cytokines (TNF-α, IFN-γ, IL-17) in tumor-infiltrated CD4+ Th1 and Th17 lymphocytes and attenuate melanoma growth and progression. In other embodiments, dMSCs are treated such that they up-regulate the secretion of perforine and granzyme B-containing vesicles from activated CTLs and NK cells, thereby enhancing their tumoricidal potential. In another embodiment, dMSCs are injected in a subject to induce the generation of an immunosuppressive phenotype in CD4+T lymphocytes and prevented the trans-differentiation of TGF-β and IL-10-producing Tregs into anti-tumorigenic IFN-γ- and IL-17-producing Th1 and Th17 cells. Regarding melanoma growth, in summary the present inventors disclose that dMSC injection during the initial phase of melanoma development engrafted in a “pro-MSC1 tumor microenvironment” results in obtaining an anti-tumorigenic MSC1 phenotype.

3. d-MAPPS Augmented T Cell-Driven Immune Response to Mammary Carcinoma

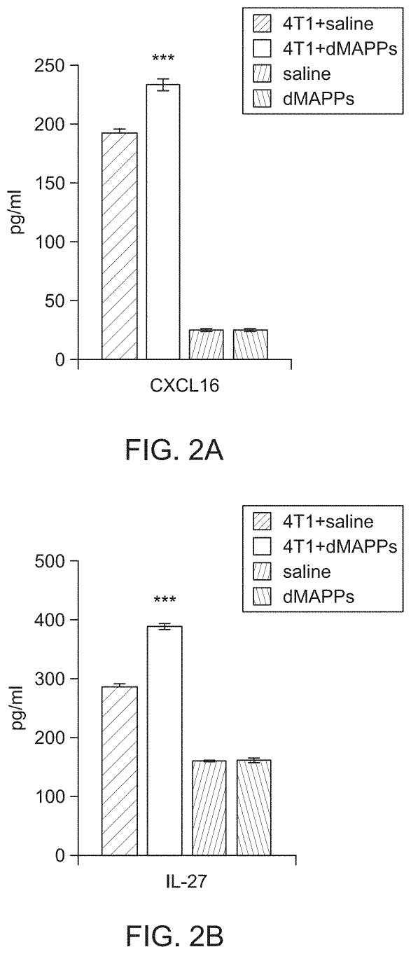

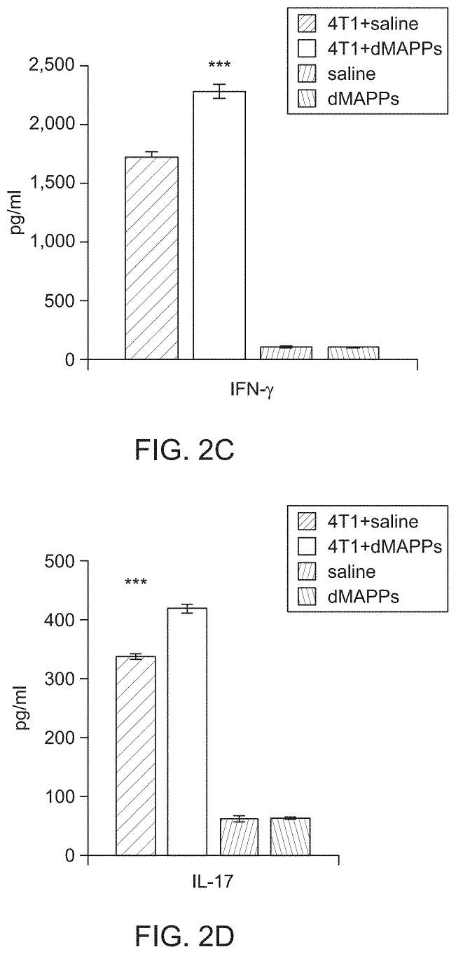

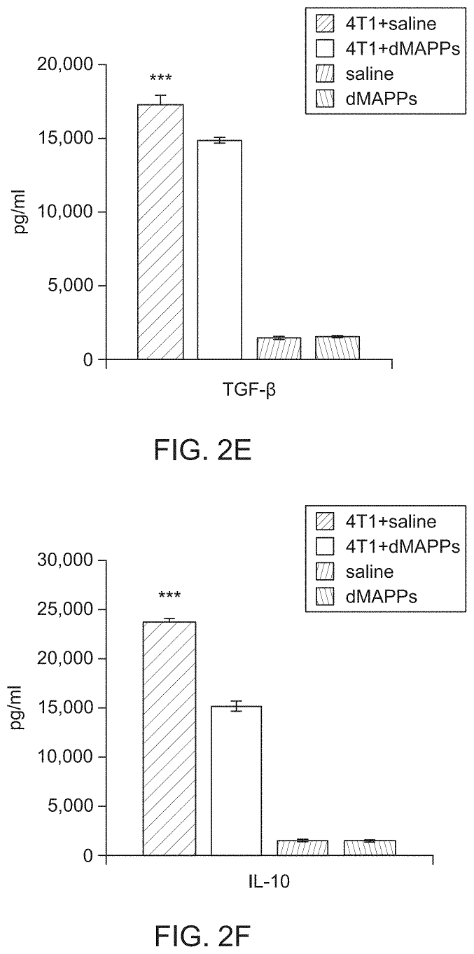

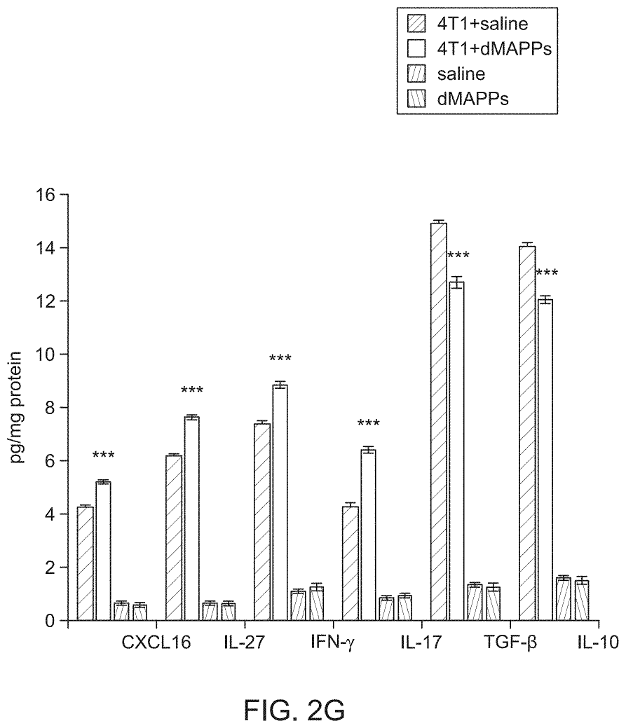

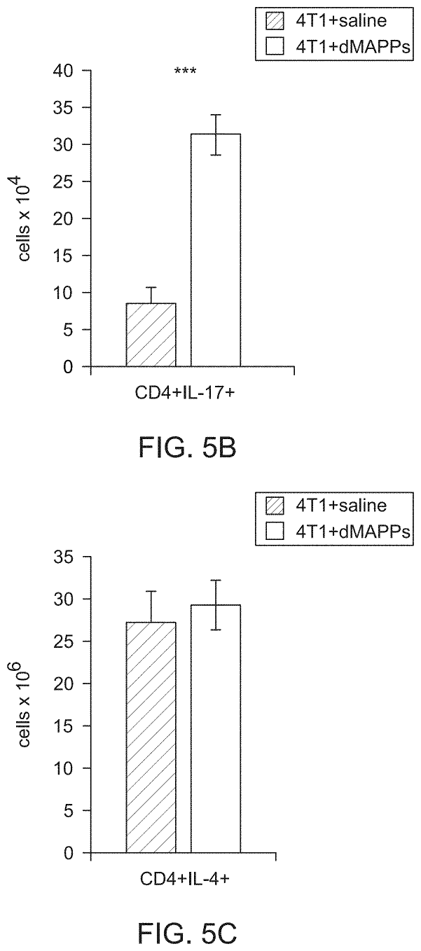

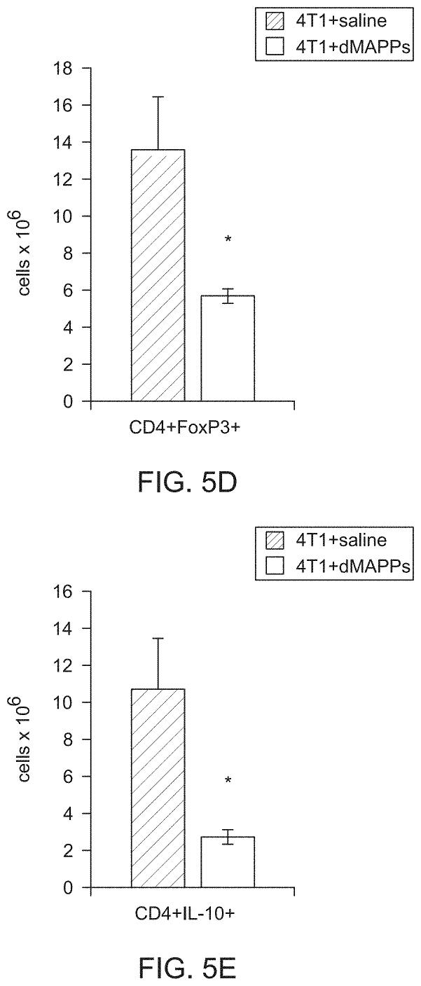

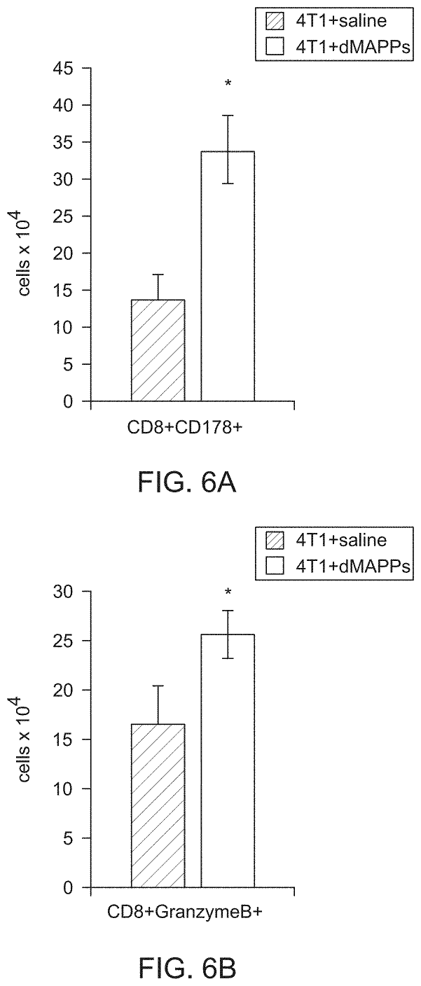

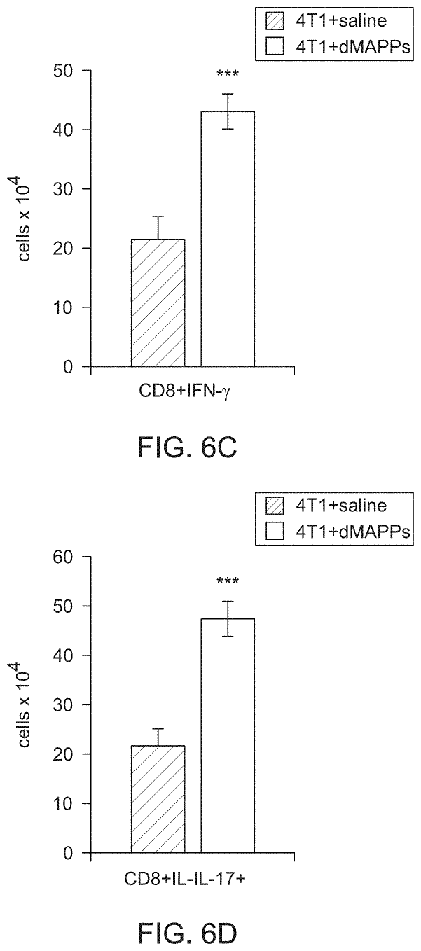

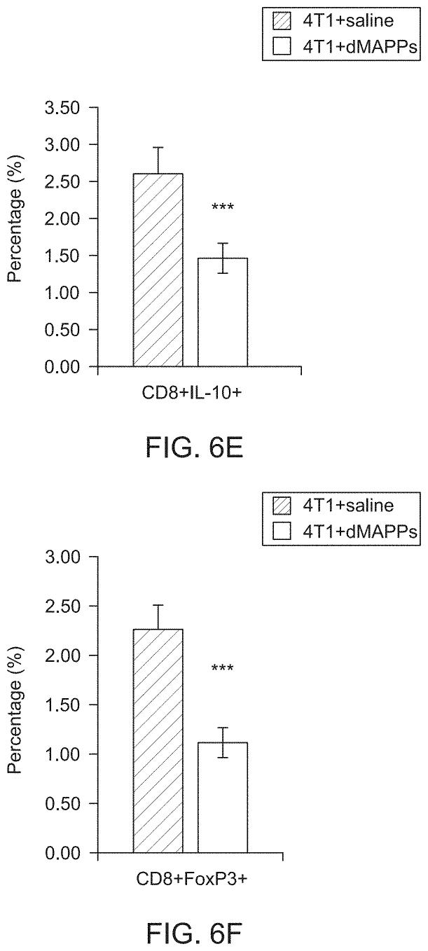

As described above, the disclosed methods for prevention and treatment of cancers (e.g. mammary carcinoma) include administering d-MAPPS to a subject, thereby altering the response of endogenous immune cells (e.g., host T cells and/or immunostimulatory molecules) in the subject. Notably, d-MAPPS™ regenerative biologics platform technology (“d-MAPPS”) contains high concentrations of exogenous immune cells that, when administered to a subject, induce and/or enhance endogenous immune cells (e.g., host T cells and/or immunostimulatory molecules) in the subject. In embodiments, said endogenous immune cells further comprise immunostimulatory molecules which promote recruitment in tumors and increase tumoricidal potential. In some embodiments, said immunostimulatory molecules comprise heterodimeric cytokines. In other embodiments, said heterodimeric cytokines include members of the IL-12 cytokine family (e.g. IL-12, IL-23, IL-27 and IL-35). In some embodiments, said immunostimulatory molecules comprise CSC chemokines (e.g., CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16, and/or CXCL17). In some embodiments, once administered to a subject with cancer or a tumor, THE CXCL16 from d-MAPPS recruits CD4 and CD8+T cells in tumors. In addition, following administration to a subject with cancer or a tumor, IL-27 from d-MAPPS boosts cytotoxicity and tumoricidal potential of T cells. In some embodiments, administering d-MAPPS to a subject recruits chemokine receptor-expressing effector T cells (e.g., CXCR6-expressing effector T cells) in tumor tissue. In other embodiments, CXC16 activates tissue resident memory T cells, thereby sustaining T cell-mediated tumor protection. As shown in FIG. 2G, in some embodiments continuous administration of d-MAPPS may increase concentrations of immunostimulatory molecules (e.g., CXCL16) in the tumor microenvironment. Further, FIGS. 5-6 show that continuous administration of d-MAPPS attracts endogenous immune cells including perforin and granzyme-expressing lymphocytes (e.g., CD8+CTLs and CD4+Th1 and Th17 lymphocytes) in the tumors of 4T1+d-MAPPStreated mice.

In some embodiments, DCs are adapted to present tumor antigens to CD4+T cells within MHC class II molecules. In other embodiments, expression of co-stimulatory molecules (e.g., CD80 and CD86) is responsible for optimal priming of CD4+T and CD8+T cells by tumor-infiltrated DCs. In one example, DC-derived TNF-α and IL-12 generate Th1 and Th17 phenotype in T cells by inducing expression of transcriptional factors T-bet and ROR-yT in STAT-1 and STAT-3-dependent manner. Notably, d-MAPPS contains high concentration of IL-27 which activates STAT-1 and STAT-3 signaling pathways in naïve T cells, facilitating DC-dependent, IL-12 and TNF-α-driven generation of Th1 and Th17 cells. Accordingly, as shown in FIG. 2D, in some embodiments continuous administration of d-MAPPS increases concentrations of IL-27 in the microenvironment of breast cancers. In other embodiments, as shown in FIGS. 5-6, continuous administration of d-MAPPS further results in an observed expansion of IFN-γ- and IL-17 producing CD4+ and CD8+Th1 cells and Th17 lymphocytes in the tumors of 4T1+d-MAPPS treated mice.

In some embodiments, administration of d-MAPPS (e.g., continuous and/or controlled administration) induces T helper cells (e.g., Th1 cells) to directly kill malignant cells via release of cytokines (e.g., TNF-α). As a result, in some embodiments TNF-related apoptosis-inducing ligand (TRAIL) is activated on the surface of tumor cells (e.g. human and/or murine mammary carcinoma tumor cells). Additionally, in some embodiments T helper cell-derived cytokines (e.g., Th1 cell-derived IFN-γ) induce enhanced production of tumorotoxic nitric oxide and TNF-α in macrophages and DCs and stimulate release of immunoglobulins (e.g., IgG, IgA, IgD, IgE, and/or IgM) from plasma cells. This enables immunoglobulin-based opsonization (e.g., IgG-based opsonization) and phagocytosis of tumor cells. In some embodiments, administration of d-MAPPS induces Th17 lymphocytes to produce pro-inflammatory cystine knot cytokines such as IL-17 (e.g., IL-17A, IL-17B, IL-17C, IL-17D, IL-17E, and/or IL-17F) and alpha helical cytokines (e.g., IL-22) that stimulate the expression of CC chemokines (e.g., CCL2 and CCL20) in tumor infiltrated macrophages and DCs. In one example, said stimulation of CC chemokines promotes the recruitment of CCR6-expressing CTLs which, in turn, utilize FasL/Fas and perforin/granzyme pathways to induce apoptosis of malignant cells.

Following continuous administration of d-MAPPS™ regenerative biologics platform technology (“d-MAPPS”), in some embodiments IL-12 cytokine (e.g. IL-12, IL-23, IL-27 and IL-35) stimulation also enhances generation and cytotoxicity of CTLs. In one example, upon binding to IL-27 receptor (“IL-27R”), IL-27 induces enhanced synthesis of the transcription factors T-bet and eomesodermin in naïve CD8+T lymphocytes, enabling their conversion in effector CD8+CTLs. In another example, following administration of d-MAPPS IL-27/IL-27R axis up-regulates production of pro-apoptotic molecules (e.g., granzyme B and perforin) in effector CD8+CTLs, enhancing their cytotoxic potential. In some embodiments, said enhanced generation and cytotoxicity of CTLs is observed in humans and in a variety of mouse tumor models (e.g., plasmacytoma, melanoma, neuroblastoma).

In some embodiments, immunosuppressive regulatory T cells (e.g., FoxP3-expressing CD4+ and CD8+ T lymphocytes) inhibit anti-tumor immunity in an IL-10 and TGF-β-dependent manner. In some embodiments, IL-27 in tumor bearing animals down-regulates expression of FoxP3 in CD4+ and CD8+ T cells and prevents generation and expansion of immunosuppressive regulatory T cells (“Tregs”). Accordingly, as shown in FIG. 2, in some embodiments the present inventors disclose that increased levels of IL-27 following administration of d-MAPPS™ regenerative biologics platform technology (“d-MAPPS”) induces a reduction in IL-10 and TGF-β-producing, FoxP3-expressing Tregs and further down-regulates serum levels of IL-10 and TGF-β (see FIG. 2E-F). In some embodiments, following administration of d-MAPPS, check point inhibitors (e.g., CTLA-4, PD-1, and PD-L1) are administered to prevent excessive exhaustion of T cells in the tumor microenvironment. In other embodiments, check point inhibitors significantly enhance systemic anti-tumor effects of d-MAPPS.

4. Neurodegenerative Diseases

In some embodiments, anti-cancer d-MAPPS formulations are used in combination with one or more agents for the treatment of associated neurodegenerative diseases or neurological dysfunction. In further embodiments, the formulations are administered with one or more agents suitable for brain injuries, dementia, or PTSD.

Active agents for the treatment of neurodegenerative diseases are well known in the art and can vary based on the symptoms and disease to be treated. For example, conventional treatment for Parkinson's disease can include levodopa (usually combined with a dopa decarboxylase inhibitor or COMT inhibitor), a dopamine agonist, or an MAO-B inhibitor. Treatment for Huntington's disease can include a dopamine blocker to help reduce abnormal behaviors and movements, or a drug such as amantadine and tetrabenazine to control movement, etc. Other drugs that help to reduce chorea include neuroleptics and benzodiazepines. Compounds such as amantadine or remacemide have shown preliminary positive results. Hypokinesia and rigidity, especially in juvenile cases, can be treated with antiparkinsonian drugs, and myoclonic hyperkinesia can be treated with valproic acid. Psychiatric symptoms can be treated with medications similar to those used in the general population. Selective serotonin reuptake inhibitors and mirtazapine have been recommended for depression, while atypical antipsychotic drugs are recommended for psychosis and behavioral problems. Said therapeutics may be safely administered in combination with anti-cancer d-MAPPS formulations for subjects suffering from both cancer and a neurodegenerative disease.

Relatedly, Riluzole (RILUTEK®) (2-amino-6-(trifluoromethoxy) benzothiazole), an antiexcitotoxin, has yielded improved survival time in subjects with ALS. Other medications, most used off-label, and interventions can reduce symptoms due to ALS. Some treatments improve quality of life and a few appear to extend life. Common ALS-related therapies are reviewed in Gordon, Aging and Disease, 4(5):295-310 (2013), see, e.g., Table 1 therein. A number of other agents have been tested in one or more clinical trials with efficacies ranging from non-efficacious to promising. Exemplary agents are reviewed in Carlesi, et al., Archives Italiennes de Biologie, 149:151-167 (2011). For example, therapies may include an agent that reduces excitotoxicity such as talampanel (8-methyl-7H-1,3-dioxolo(2,3)benzodiazepine), a cephalosporin such as ceftriaxone, or memantine; an agent that reduces oxidative stress such as coenzyme Q10, manganoporphyrins, KNS-760704 [(6R)-4,5,6,7-tetrahydro-N6-propyl-2,6-benzothiazole-diamine dihydrochloride, RPPX], or edaravone (3-methyl-1-phenyl-2-pyrazolin-5-one, MCI-186); an agent that reduces apoptosis such as histone deacetylase (HDAC) inhibitors including valproic acid, TCH346 (Dibenzo(b,f)oxepin-10-ylmethyl-methylprop-2-ynylamine), minocycline, or tauroursodeoxycholic Acid (TUDCA); an agent that reduces neuroinflammation such as thalidomide and celastol; a neurotropic agent such as insulin-like growth factor 1 (IGF-1) or vascular endothelial growth factor (VEGF); a heat shock protein inducer such as arimoclomol; or an autophagy inducer such as rapamycin or lithium.

Treatment for Alzheimer's Disease in can include, for example, an acetylcholinesterase inhibitor such as tacrine, rivastigmine, galantamine or donepezil; an NMDA receptor antagonist such as memantine; or an antipsychotic drug.

Treatment for Dementia with Lewy Bodies can include, for example, acetylcholinesterase inhibitors such as tacrine, rivastigmine, galantamine or donepezil; the N-methyl d-aspartate receptor antagonist memantine; dopaminergic therapy, for example, levodopa or selegiline; antipsychotics such as olanzapine or clozapine; REM disorder therapies such as clonazepam, melatonin, or quetiapine; anti-depression and antianxiety therapies such as selective serotonin reuptake inhibitors (citalopram, escitalopram, sertraline, paroxetine, etc.) or serotonin and noradrenaline reuptake inhibitors (venlafaxine, mirtazapine, and bupropion) (see, e.g., Macijauskiene, et al., Medicina (Kaunas), 48(1):1-8 (2012)).

Exemplary neuroprotective agents are also known in the art and include, for example, glutamate antagonists, antioxidants, and NMDA receptor stimulants. Other neuroprotective agents and treatments include caspase inhibitors, trophic factors, anti-protein aggregation agents, therapeutic hypothermia, and erythropoietin.

Other common active agents for treating neurological dysfunction include amantadine and anticholinergics for treating motor symptoms, clozapine for treating psychosis, cholinesterase inhibitors for treating dementia, and modafinil for treating daytime sleepiness. As described above, said therapeutics may be safely administered in combination with anti-cancer d-MAPPS formulations for subjects suffering from both cancer and a neurodegenerative disease.

5. Antimicrobial Agents

In some embodiments, d-MAPPS formulations are used in combination with one or more antimicrobial agents. An antimicrobial agent is a substance that kills or inhibits the growth of microbes such as bacteria, fungi, viruses, or parasites. Antimicrobial agents include antiviral agents, antibacterial agents, antiparasitic agents, and anti-fungal agents. Representative antiviral agents include ganciclovir and acyclovir. Representative antibiotic agents include aminoglycosides such as streptomycin, amikacin, gentamicin, and tobramycin, ansamycins such as geldanamycin and herbimycin, carbacephems, carbapenems, cephalosporins, glycopeptides such as vancomycin, teicoplanin, and telavancin, lincosamides, lipopeptides such as daptomycin, macrolides such as azithromycin, clarithromycin, dirithromycin, and erythromycin, monobactams, nitrofurans, penicillins, polypeptides such as bacitracin, colistin and polymyxin B, quinolones, sulfonamides, and tetracyclines.

Other exemplary antimicrobial agents include iodine, silver compounds, moxifloxacin, ciprofloxacin, levofloxacin, cefazolin, tigecycline, gentamycin, ceftazidime, ofloxacin, gatifloxacin, amphotericin, voriconazole, natamycin. As described above, said antimicrobial agents may be safely administered as a component of anti-microbial d-MAPPS™ regenerative biologics platform technology (“d-MAPPS”) formulations to subjects with cancer and/or a microbial infection. In other embodiments, said antimicrobial agents may be safely administered as a component of anti-microbial d-MAPPS formulations for the prevention of cancer, tumors, and microbial infections.

Other antiproliferative agents include, but are not limited to, rapamycin and its analogs, including everolimus, zotarolimus, tacrolimus, novolimus, and pimecrolimus, angiopeptin, angiotensin converting enzyme inhibitors, such as captopril, cilazapril or lisinopril, calcium channel blockers, such as nifedipine, amlodipine, cilnidipine, lercanidipine, benidipine, trifluperazine, diltiazem and verapamil, fibroblast growth factor antagonists, fish oil (omega 3-fatty acid), histamine antagonists, lovastatin, topoisomerase inhibitors, such as etoposide and topotecan, as well as antiestrogens such as tamoxifen.

6. Local Anesthetics

In some embodiments, d-MAPPS formulations are used in combination with one or more local anesthetics. A local anesthetic is a substance that causes reversible local anesthesia and has the effect of loss of the sensation of pain. Non-limiting examples of local anesthetics include ambucaine, amolanone, amylocaine, benoxinate, benzocaine, betoxycaine, biphenamine, bupivacaine, butacaine, butamben, butanilicaine, butethamine, butoxycaine, carticaine, chloroprocaine, cocaethylene, cocaine, cyclomethycaine, dibucaine, dimethysoquin, dimethocaine, diperodon, dycyclonine, ecgonidine, ecgonine, ethyl chloride, etidocaine, beta-eucaine, euprocin, fenalcomine, formocaine, hexylcaine, hydroxytetracaine, isobutyl p-aminobenzoate, leucinocaine mesylate, levoxadrol, lidocaine, mepivacaine, meprylcaine, metabutoxycaine, methyl chloride, myrtecaine, naepaine, octacaine, orthocaine, oxethazaine, parethoxycaine, phenacaine, phenol, piperocaine, piridocaine, polidocanol, pramoxine, prilocaine, procaine, propanocaine, proparacaine, propipocaine, propoxycaine, psuedococaine, pyrrocaine, ropivacaine, salicyl alcohol, tetracaine, tolycaine, trimecaine, zolamine, and any combination thereof. In other aspects of this embodiment, the d-MAPPS formulations comprises an anesthetic agent in an amount of, e.g., about 10 mg, about 50 mg, about 100 mg, about 200 mg, or more than 200 mg. The concentration of local anesthetics in the compositions can be therapeutically effective meaning the concentration is adequate to provide a therapeutic benefit without inflicting harm to the patient. In some embodiments, said anesthetics may be safely administered as a component of a d-MAPPS formulation to subjects requiring anesthesia.

7. Anti-Inflammatory Agents

In some embodiments, d-MAPPS™ regenerative biologics platform technology (“d-MAPPS”) formulations are used in combination with one or more anti-inflammatory agents. Anti-inflammatory agents reduce inflammation and include steroidal and non-steroidal drugs. Suitable steroidal active agents include glucocorticoids, progestins, mineralocorticoids, and corticosteroids. In some embodiments, d-MAPPS formulations are used in combination with one or more corticosteroids.

Exemplary anti-inflammatory agents include triamcinolone acetonide, fluocinolone acetonide, methylprednisolone, prednisolone, dexamethasone, loteprendol, fluorometholone, ibuprofen, aspirin, colchicine and glucocorticoids, such as betamethasone, cortisone, dexamethasone, budesonide, prednisolone, methylprednisolone and hydrocortisone and naproxen. Exemplary immune-modulating drugs include cyclosporine, tacrolimus and rapamycin. Exemplary non-steroidal anti-inflammatory drugs (NSAIDs) include mefenamic acid, aspirin, Diflunisal, Salsalate, Ibuprofen, Naproxen, Fenoprofen, Ketoprofen, Deacketoprofen, Flurbiprofen, Oxaprozin, Loxoprofen, Indomethacin, Sulindac, Etodolac, Ketorolac, Diclofenac, Nabumetone, Piroxicam, Meloxicam, Tenoxicam, Droxicam, Lornoxicam, Isoxicam, Meclofenamic acid, Flufenamic acid, Tolfenamic acid, elecoxib, Rofecoxib, Valdecoxib, Parecoxib, Lumiracoxib, Etoricoxib, Firocoxib, Sulphonanilides, Nimesulide, Niflumic acid, and Licofelone.

In some embodiments, anti-inflammatory agents are anti-inflammatory cytokines. Exemplary cytokines are IL-10, IL-17, TNF-α, TGF-β and IL-35. Anti-inflammatory cytokines in the context of biomaterial tumor site, skin grafts, and hair grafts are cytokine that induce an anti-inflammatory immune environment or suppress inflammatory immune environment. Activation of regulatory T cells, Tregs, is involved in the prevention of rejection, the induction and maintenance of peripheral tolerance of the allograft. Th17 cells are a subset of T helper cells which is characterized by the production of IL-17. Th17 cells have been suggested to play a role in allograft rejection. In some embodiments, cytokines to be added to the amniotic fluid formulations are those that induce Tregs activation (e.g. IL-25) and suppress Th17 activation (e.g. IL-10) for minimizing rejection. As described above, said anti-inflammatory molecules may be safely co-administered with d-MAPPS to subjects experiencing local or systemic inflammation. In other embodiments, said anti-inflammatory molecules may be safely administered as a component of d-MAPPS for the prevention of cancer-associated inflammation, tumors, and other disorders described herein.

8. Immune Cells and Markers

In some embodiments, the amniotic fluid formulation further comprises at least one eukaryotic cell type. Some exemplary eukaryotic cell types include stem cells, immune cells such as T lymphocytes, B lymphocytes, natural killer cells, leukocytes, and dendritic cells, or combinations thereof. In some embodiments, the above-described cells are tumor-infiltrated cells (e.g., tumor-infiltrated macrophages, tumor-infiltrated natural killer cells, 41T-treated cells, and the like). Notably, endogenous immune cells refer to cells generate from a subject, whereas exogenous immune cells refer to immune cells introduced from an external source. For example, d-MAPPS itself contains exogenous immune cells which, when administered to a subject, stimulate endogenous immune cells in the subject.

In some embodiments, the stem cells are mesenchymal stem cells. Functional characteristics of mesenchymal stem cells that may benefit wound healing include their ability to migrate to the site of injury or inflammation, participate in regeneration of damaged tissues, stimulate proliferation and differentiation of resident progenitor cells, promote recovery of injured cells through growth factor secretion and matrix remodeling, and exert unique immunomodulatory and anti-inflammatory effects.

In certain embodiments, the mesenchymal stem cells protect target/injured tissue through suppression of pro-inflammatory cytokines, and through triggering production of reparative growth factors. In some embodiments, the d-MAPPS further comprises at least one immune molecule (e.g., anti-inflammatory markers, inflammatory factors, cytokines, regulatory proteins, glycoproteins, costimulatory molecules, growth factors, and the like).

In some embodiments prevention and/or treatment of cancers and tumors via d-MAPPS administration alters the total number of endogenous immune cells in a breast cancer model. For example, prevention and/or treatment of cancers and tumors via d-MAPPS administration may alter (e.g., enhance or induce) the total number tumor-infiltrated leukocytes in a breast cancer. In another example, d-MAPPS may or may not alter the number of tumor-infiltrated F4/80+ macrophages in a breast cancer or other tumor. In other embodiments, d-MAPPS administration alters the total number of endogenous immune cells in a tumor (e.g., a cancerous tumor, a non-cancerous tumor, and the like).

In some embodiments, administration of d-MAPPS to a subject for the prevention and/or treatment of cancers and/or tumors may or may not alter (e.g., enhances, induces, decreases, etc.) the concentration of endogenous immune cells in the following exemplary manner: 1) altering the total number of tumor-infiltrated leukocytes in breast cancer, 2) altering the number of tumor-infiltrated F4/80+ macrophages in breast cancer, 3) altering the number of CD80+ cells in tumor-infiltrated F4/80+ macrophages, 4) altering the number of endogenous immune cells or immune molecules in a tumor (e.g., a cancerous tumor, a non-cancerous tumor, and the like), 5) altering the total number of tumor-infiltrated CD80+F4/80+ macrophages in breast cancer, 6) altering the percentage of CD86+ cells in tumor-infiltrated F4/80+ macrophages, 7) altering the total number of tumor-infiltrated CD86+F4/80+ macrophages in breast cancer, 8) altering the percentage of CD80+CD86+ cells in tumor-infiltrated F4/80+ macrophages, 9) altering the total number of tumor-infiltrated CD80+CD86+F4/80+ macrophages in breast cancer, 10) altering the percentage of I-A+ cells in tumor-infiltrated F4/80+ macrophages, 11) altering the total number of tumor-infiltrated I-A+F4/80+ macrophages in breast cancer, 12) altering the percentage of TNF alpha+ cells in tumor-infiltrated F4/80+ macrophages, 13) altering the total number of tumor-TNF alpha+F4/80+ macrophages in breast cancer, 14) altering the percentage of IL-12+ cells in tumor-infiltrated F4/80+ macrophages, 15) altering the total number of tumor-infiltrated IL-12+F4/80+ macrophages in breast cancer, 16) altering the percentage of IL-10+ cells in tumor-infiltrated F4/80+ macrophages, 17) altering the total number of tumor-infiltrated IL-10+F4/80+ macrophages, 18) altering the percentage of tumor-infiltrated CD11c+ dendritic cells (DC) in breast cancer, 19) altering the total number of tumor-infiltrated CD11c+ DCs in breast cancer, 20) altering the percentage of CD80+ cells in tumor-infiltrated CD11c+ DCs, 21) altering the total number of tumor-infiltrated CD80+CD11c+ DCs in breast cancer, 22) altering the percentage of CD86+ cells in tumor-infiltrated CD11c+ DCs, 23) altering the total number of tumor-infiltrated CD86+CD11c+ DCs in breast cancer, 24) altering the percentage of CD80+CD86+ cells in tumor-infiltrated CD11c+ DCs, 25) altering the total number of tumor-infiltrated CD80+CD86+CD11c+ DCs in breast cancer, 26) altering the percentage of I-A+ cells (e.g., I-A alloantigen) in tumor-infiltrated CD11c+ DCs, 27) altering the total number of tumor-infiltrated I-A+CD11c+ DCs in murine breast cancer, 28) altering the percentage of TNF alpha+ cells in tumor-infiltrated CD11c+ DCs, 29) altering the total number of tumor-TNF alpha+CD11c+ DCs in breast cancer, 30) altering the percentage of IL-12+ cells in tumor-infiltrated CD11c+ DCs, 31) altering the total number of tumor-infiltrated IL-12+CD11c+ DCs in breast cancer, 32) altering the percentage of IL-10+ cells in tumor-infiltrated CD11c+ DCs, 33) altering the total number of tumor-infiltrated IL-10+CD11c+ DCs in breast cancer, 34) altering the percentage of tumor-infiltrated CD4+ T cells in breast cancer, 35) altering the total number of tumor-infiltrated CD4+ T cells in breast cancer, 36) altering the percentage of IL-10+ cells in tumor-infiltrated CD4+ T cells in breast cancer, 37) altering the total number of tumor-infiltrated CD4+IL-10+ T cells in breast cancer, 38) altering the percentage of FoxP3+ cells in tumor-infiltrated CD4+ T cells in breast cancer, 39) altering the total number of tumor-infiltrated CD4+FoxP3+T regulatory (Treg) cells in breast cancer, 40) altering the percentage of TNFalpha+ cells in tumor-infiltrated CD4+ T cells in breast cancer, 41) altering the total number of tumor-infiltrated CD4+TNF alpha+ T cells in breast cancer, 42) altering the percentage of IL-4+ cells in tumor-infiltrated CD4+ T cells, 43) altering the total number of tumor-infiltrated CD4+IL-4+ Tcells in breast cancer, 44) altering the percentage of IL-17+ cells in tumor-infiltrated CD4+ T cells in breast cancer, 45) altering the total number of tumor-infiltrated CD4+IL-17+ T cells in breast cancer, 46) altering the percentage of IFN-γ+ cells in tumor-infiltrated CD4+ T cells in breast cancer, 47) altering the total number of tumor-infiltrated CD4+IFN-γ+ T cells in breast cancer, 48) altering the percentage of tumor-infiltrated CD8+ T cells in breast cancer, 49) altering the total number of tumor-infiltrated CD8+ T cells in breast cancer, 50) altering the percentage of IL-10+ cells in tumor-infiltrated CD8+ T cells in breast cancer, 51) altering the total number of tumor-infiltrated CD8+IL-10+ T cells in breast cancer, 52) altering the percentage of FoxP3+ cells in tumor-infiltrated CD8+ T cells in breast cancer, 53) altering the total number of tumor-infiltrated CD8+FoxP3+ regulatory cells in breast cancer, 54) altering the percentage of TNF alpha+ cells in tumor-infiltrated CD8+ T cells in breast cancer, 55) altering the total number of tumor-infiltrated CD8+ TNFalpha+ T cells in breast cancer, 56) altering the percentage of IL-4+ cells in tumor-infiltrated CD8+ T cells, 57) altering the total number of tumor-infiltrated CD8+IL-4+ T cells in breast cancer, 58) altering the percentage of IL-17+ cells in tumor-infiltrated CD8+ T cells in breast cancer, 59) altering the total number of tumor-infiltrated CD8+IL-17+ T cells in breast cancer, 60) altering the percentage of IFN-γ+ cells in tumor-infiltrated CD8+ T cells in breast cancer, 61) altering the total number of tumor-infiltrated CD8+IFN-γ+ T cells in breast cancer, 62) altering the percentage of CD178+ cells in tumor-infiltrated CD8+ T cells in breast cancer, 63) altering the total number of tumor-infiltrated CD8+CD178+ T cells in breast cancer, 64) altering the percentage of Granzyme B+ cells in tumor-infiltrated CD8+ T cells in breast cancer, 65) altering the total number of tumor-infiltrated CD8+Granzyme B+ T cells in breast cancer, 66) altering the percentage of tumor-infiltrated NK1.1+NK cells in breast cancer, 67) altering the total number of tumor-infiltrated NK1.1+NK cells in breast cancer, 68) altering the percentage of CD178+ cells in tumor-infiltrated NK1.1+NK cells in breast cancer, 69) altering the total number of tumor-infiltrated NK1.1+CD178+NK cells in breast cancer, 70) altering the percentage of Granzyme B+ cells in tumor-infiltrated NK1.1+NK cells in breast cancer, 71) altering the total number of tumor-infiltrated NK1.1+Granzyme B+NK cells in breast cancer, 72) altering the percentage of IL-17+ cells in tumor-infiltrated NK1.1+NK cells in breast cancer, 73) altering the total number of tumor-infiltrated NK1.1+IL-17+NK cells in breast cancer, 74) altering the percentage of IFN-γ+ cells in tumor-infiltrated NK1.1+NK cells in breast cancer, and/or 75) altering the total number of tumor-infiltrated NK1.1+IFN-γ+NK cells in breast cancer.

In some aspects, a particular immune cell, immunostimulatory molecule, chemokine, glycoprotein, and/or protein small molecule is isolated from d-MAPPS (e.g. by column separation), amplified, and administered to a subject in isolation as a therapeutic and/or secondary anti-cancer therapeutic. Said isolates may be derivatized by standards means (e.g. derivatizing a small molecule to improve solubility, derivatizing a surface glycoprotein to enable targeting, and the like). In other embodiments, a synthetic route is contemplated to construct said isolates using standard biochemical practices (e.g., an organic synthetic route, or enzymatic protein construction). For example, the present inventors contemplate enzymatic and/or synthetic construction of IL-27, IL-17, and CXC16, the key immunostimulatory molecules that are present at high concentrations in purified dMSC-derived d-MAPPS and which are known to down-regulate immunosuppressive endogenous immune proteins IL-10 and TFG-β in the tumor microenvironment of a subject with breast cancer.

9. Stem Cell Exosomes

In some embodiments, the exosomes present in d-MAPPS composition is effective to treat one or more disclosed diseases or disorders. Thus, methods of preparing sterile de-cellularized amniotic fluid is optimized to maximize the number of exosomes present in the raw amniotic fluid, for example, 90%, 80%, 70%, 60%, 50%, 40%, or more than 30%.

In some embodiments, high concentrations of exosomes are required for effective treatment of one or more diseases or disorders (e.g., cancers, tumor, and the like). In these cases, stem cells are induced to produce an increased amount of exosomes. Exemplary methods for induction of exosomes (“Exos”) from stem cells include treatment of the stem cells with cytokines, treatment with liposome stimulation using one or more stimulant liposomes such as neutral or cationic liposomes (Emam S E et al., Biol Pharm Bull. 2018; 41(5):733-742), or other physical and/or biological methods previously described (Phan J et al., J Extracell Vesicles. 2018; 7(1): 1522236).

Generally, methods of isolating exosomes are known including one or more of differential ultracentrifugation-based techniques, size-based techniques, immunoaffinity capture-based techniques, exosome precipitation, and microfluidics-based techniques (Li P et al., Theranostics. 2017; 7(3): 789-804).

In some embodiments, the d-MAPPS formulations comprise exogenous exosomes generated ex vivo from amniotic fluid dMSCs, or derived from dMSCs of other sources.

10. Other Agents

In some embodiments, d-MAPPS formulations are used in combination with one or more other agents, such as growth factors. Growth factors (e.g., cytokines), refer to proteins and/or glycoproteins capable of stimulating cellular growth, proliferation, and/or cellular differentiation. Non-limiting examples of growth factors include transforming growth factor beta (TGF-0), transforming growth factor alpha (TGF-α), granulocyte-colony stimulating factor (GCSF), granulocyte-macrophage colony stimulating factor (GM-CSF), nerve growth factor (NGF), neurotrophins, platelet-derived growth factor (PDGF), erythropoietin (EPO), thrombopoietin (TPO), myostatin (GDF8), growth differentiation factor-9 (GDF9), acidic fibroblast growth factor (aFGF or FGF-1), basic fibroblast growth factor (bFGF or FGF-2), epidermal growth factor (EGF), vascular endothelial growth factor (VEGF) and hepatocyte growth factor (HGF).

In some embodiments, the d-MAPPS formulation (e.g., d-MAPPS pharmaceutical formulation or d-MAPPS pharmaceutical composition) can include antibodies, including, for example, daclizumab, bevacizumab (AVASTIN®), ranibizumab (LUCENTIS®), basiliximab, ranibizumab, and pegaptanib sodium or peptides like SN50, and antagonists of NF. Other antibodies can be used as well including Muromonab-DC3, Satumomab, Abciximab, Nofetumomab, Imiciromab, Capromab, Arcitumomab, Rituximab, Daclizumab, Basiliximab, Infliximab, Palivizumab, Trastuzumab, Gemtuzumab, Adalimumab, Alemtuzumab, Ibritumomab tiuxetan, Efalizumab, Idarucizumab, Evolocumab, Daratumumab, Mepolizumab, Necitumumab, Obiltoxaximab, Ixekizumab, Reslizumab, Atezolizumab, Daclizumab, Adalimumab-Atto, Ustekinumab, Atezolizumab, Olaratumab, Bezlotoxumab, Denosumab, Belimumab, Brentuximab, Denosumab, Ipilimumab, Pertuzumab, Raxibacumab, Obinutuzumab, Trastuzumab, Siltuximab, Ramucirumab, Blinatumomab, Pembrolizumab, Vedolizumab, Secukinumab, Nivolumab, Dinutuximab, Omalizumab, Tositumomab, Bevacizumab, Cetuximab, Fanolesomab, Catumaxomab, Panitumumab, Ranibizumab, Eculizumab, Certolizumab pegol, Canakinumab, Golimumab, Ofatumumab, Tocilizumab, Ustekinumab

In further embodiments, the formulation can include oligonucleotides. Representative oligonucleotides include siRNAs, microRNAs, DNA, and RNA. Oligonucleotides can be used as gene therapy complementing the efficacy of the amniotic fluid formulations.

In some embodiments, the d-MAPPS formulation (e.g., d-MAPPS pharmaceutical formulation or d-MAPPS pharmaceutical composition) further comprises one or more enzyme cofactors, and/or one or more essential nutrients. Exemplary cofactors include vitamin C, biotin, vitamin E, and vitamin K. Exemplary essential nutrients are amino acids, fatty acids, etc.

In other embodiments, an additional suitable therapeutic agent (or “drug”) may be incorporated into, coated on, or otherwise combined in various embodiments. Examples of such therapeutic agents include, but are not limited to, antiproliferatives, anti-inflammatories, agents that inhibit hyperplasia, smooth muscle cell inhibitors, antibiotics, growth factor inhibitors, cell adhesion inhibitors, cell adhesion promoters, antimitotics, antifibrins, antioxidants, anti-neoplastics, agents that promote endothelial cell recovery, matrix metalloproteinase inhibitors, anti-metabolites, antiallergic substances, viral vectors, nucleic acids, monoclonal antibodies, inhibitors of tyrosine kinase, antisense compounds, oligonucleotides, cell permeation enhancers, hypoglycemic agents, hypolipidemic agents, proteins, nucleic acids, anti-nauseants/anti-emetics, PPAR alpha agonists such as fenofibrate, PPAR-gamma agonists selected such as rosiglitazaone and pioglitazone, sodium heparin, LMW heparins, heparoids, hirudin, argatroban, forskolin, vapriprost, prostacyclin and prostacylin analogues, dextran, D-phe-pro-arg-chloromethylketone (synthetic anti-thrombin), glycoprotein IIb/IIIa (platelet membrane receptor antagonist antibody), recombinant hirudin, thrombin inhibitors, indomethacin, phenyl salicylate, beta-estradiol, vinblastine, ABT-627 (astrasentan), testosterone, progesterone, paclitaxel, methotrexate, fotemusine, RPR-101511A, cyclosporine A, vincristine, carvediol, vindesine, dipyridamole, methotrexate, folic acid, thrombospondin mimetics, estradiol, dexamethasone, metrizamide, iopamidol, iohexol, iopromide, iobitridol, iomeprol, iopentol, ioversol, ioxilan, iodixanol, and iotrolan, antisense compounds, inhibitors of smooth muscle cell proliferation, lipid-lowering agents, radiopaque agents, antineoplastics, HMG CoA reductase inhibitors such as lovastatin, atorvastatin, simvastatin, pravastatin, cerivastatin and fluvastatin, and combinations thereof. Solutions, suspensions, or emulsions may also contain one or more excipients known in the art, such as dispersing agents, wetting agents, and suspending agents.

B. Storage

In some embodiments, d-MAPPS filtrate can be stored in frozen condition at about −20° C. to about −80° C. for long-term storage. In addition, the sterilely filtered amniotic fluid may be distributed in vials equipped with special rubber stoppers for sterile lyophilization.

The lyophilization is carried out in a sterile environment. The rubber stoppers on the vials are then automatically pushed down in the freeze dryer to definitively close them. Then an aluminum cap is sealed on each vial to protect its sterile content. In such a lyophilized state, the amniotic fluid may be stored at +4° C. or room temperature for at least one year without decrease of its biological activity. For its medical use, the sterile amniotic fluid may be reconstituted by adding the initial volume of sterile water to the powder in order to restore a transparent and homogeneous physiological liquid.

The decellularization and purification process protects the growth factors and other biological components of amniotic fluid from chemical and enzymatic degradation. Molecules contained within the fluid are stabilized against degradation, avoiding the need for chemical or physical modification to maintain the biological activity of the molecules over extended periods of time. Therefore, d-MAPPS prepared according to the described methods can be stored for long periods of time, allowing for a broad range of application methods, including distribution and storage as aerosols, solutions, powders, etc.

In some embodiments, the sterile d-MAPPS is refrigerated at about 1° C. to about 10° C. for long-term storage. In a further embodiment, the sterile d-MAPPS is refrigerated at 4° C. for up to 12 months and more. In yet another embodiment, the sterile d-MAPPS is stored at room temperature for over a week, 2 weeks, 3 weeks, a month, 2 months, 3 months, 6 months, up to 12 months and more while retain most biologically active components, preferably comparable to those in the d-MAPPS refrigerated at about 1° C. to about 10° C. for a similar duration. For example, fluids purified according to the described methods retain the biological properties of the component molecules over extended periods of storage, ideally without the need for freeze/thawing.

Preferably, the long-term storage does not reduce the quantity of the total soluble proteins or factors present in the d-MAPPS. For some embodiments, the total soluble proteins retained after long-term storage in frozen, refrigerated or room temperature conditions is about 10%, 20%, 30%, 40%, 50%, 60%, 70%, 80%, or 90% of the fresh d-MAPPS.

The protein quantities remaining soluble in d-MAPPS after a period of storage is assessed by common protein quantification methods such as bicinchoninic acid (BCA) assay, Bradford assay, Lowry assay, and ultraviolet absorption (at 280 nm). To quantify individual proteins, high-throughput methods such as high-density screening arrays (RayBiotech, Norcross Ga.) are used.

Further, the storage does not reduce, prevent or otherwise alter the biological activity of any one or more of the amniotic factors of the DHAF. For example, in some embodiments, the biological activity of one or more amniotic factors is retained throughout storage for extended periods of time. The activity of any one or more of the amniotic growth factors of the stored product can be assessed as a % compared to that of the fresh (raw) product, or compared to the d-MAPPS prior to storage. Therefore, in some embodiments, little or no statistically significant changes in the biological activity of the amniotic factors are observed when using d-MAPPS stored at 4° C. or at room temperature for up to a day, 2 days, 3 days, 4 days, 5 days, 6 days, up to one week, up to 2 weeks, up to 3 weeks, up to 4 weeks, up to one month, up to 2 months, up to 3 months, up to 4 months, up to 5 months, up to 6 months or more than 6 months. In other embodiments, the activity of any one of the proteins in the amniotic fluid are reduced by 50%, 40%, 30%, 20%, 10%, 5%, or less than 5% relative to the raw amniotic fluid prior to the de-cellularizing process.

In some embodiments, one or more of the growth factors is reduced after storage. For example, such growth factors include FGF7, MMP-9, GCSF, MMP-7, MMP-13, TGF-β, FGF-4, EG-VEGF and IL-8. In other embodiments, one or more of the growth factors is reduced after freeze/thawing. For example, such growth factors include FGF-21, ANG2, GDNF, FGF-19, TIMP-2, ANG-1, and M-CSF. In a preferred embodiment, one or more of the growth factors is increase compared to the fresh d-MAPPS, presumably due to enhanced stability at these storage conditions. Some exemplary growth factors include VEGF-α, TNF-α, and HGF. In a further embodiment, variable changes in the growth factors such as angiotensinogen, PDGF-AA, TGF-α, EGF and SCF.

In some embodiments, inflammatory markers are decreased after refrigeration at 2-8° C. or at room temperature for one, two, three or four weeks. For example, the amount of one or more of the inflammatory proteins present in the refrigerated sample is reduced by about 1%, 5%, 10%, 20%, 30%, 40%, 50%, 60% 70%, or 80% compared to that of the fresh (raw) product. Some exemplary inflammatory markers include Eotaxin-2, IL-6, CCL18, total GRO, CXCLS, 6Ckine, and MIP-3α.

In some embodiments, inflammatory proteins are decreased after freezing. For example, the amount of one or more of the inflammatory proteins present in the sample stored in frozen condition at about −20° C. to about −80° C. is reduced by about 5%, 10%, 20%, 30%, 40%, 50%, 60% 70%, 80% or 90% compared to that of the fresh (raw) product. Some exemplary inflammatory markers include IL-la, CXCL9, MIP-la, and CCL5. In other embodiments, inflammatory markers are increased after being stored for long-term either in refrigerated or frozen conditions. Some exemplary inflammatory markers include TNF-α, MIP-10, and MCP-2.

In a preferred embodiment, anti-inflammatory molecules are not significantly decreased after being stored refrigerated or frozen for one or more days, weeks or months. In another embodiment, one or more of the anti-inflammatory molecules is decreased in d-MAPPS following a period of refrigeration. Some exemplary anti-inflammatory molecules include IL-8, IL-13, IL-27, CTLA-4, and IL-21. In another embodiment, one or more of the anti-inflammatory molecules is decreased in the d-MAPPS stored in frozen conditions. Some exemplary anti-inflammatory molecules include IL-1Ra and TGFβ1.

A. Dosages and Dosing Regimens

In embodiments, precise amount of composition to be administered is determined by an attending physician. Said physician considers various factors including age, weight, tumor size, extent of metastasis, and general health condition of the subject. Dosage and dosing regimens are dependent on the severity and location of the cancer, tumor, or other disorder. In addition, dosage and dosing regimens are dependent on the method of administration. In one example, the herein disclosed d-MAPPS pharmaceutical composition is administered at a dosage of (0.05-0.2 mL/intraperitoneal injection (ip.)/day; 4T1+saline-treated mice) or for d-MAPPS (4T1+d-MAPPS; 0.05-0.2 mL/ip./day).

In some embodiments, mice from control groups receive only saline or d-MAPPS (0.1 ml/ip./day). about 10⁴ to 10⁹ cells/kg body weight (for example, about 10⁴, 10⁵, 10⁶, 10⁷, 10⁸, or 10⁹ cells/kg), such as about 10⁴ to 10⁶ cells/kg, about 10⁵ to 10⁷ cells/kg, or about 10⁶ to 10⁸ cells/kg. In some embodiments, exemplary doses are about 10⁵ cells/kg to about 10⁹ cells/kg, such as about 10⁶ cells/kg, about 5×10⁶ cells/kg, about 10⁷ cells/kg, about 5×10⁷ cells/kg, about 10⁸ cells/kg, or about 5×10⁸ cells/kg. The population of modified T cells or NK cells is typically administered parenterally, for example intravenously; however, injection or infusion to a tumor or close to a tumor (local administration) or administration to the peritoneal cavity can also be used. One of skill in the art can determine appropriate routes of administration.

In some embodiments, disclosed d-MAPPS™ regenerative biologics platform technology (“d-MAPPS”) formulations are optimized/tailored for an individual subject, and vary depending on the nature of the condition to be treated. For example, prophylactic administration of d-MAPPS may include administration of d-MAPPS at up to ½ of the total volume administered to a patient with existing cancer. In one embodiment, d-MAPPS is formulated in a dosage between about 0.1 ml and about 100 ml, inclusive; or between about 0.1 ml and 1 ml, inclusive; or between about 1 ml and about 10 ml, inclusive; or between about 10 ml and about 50 ml, inclusive. In yet another embodiment, the formulation is combined with any amount of between about between about 0.1 ml and about 100 ml, inclusive; or between about 0.1 ml and 1 ml, inclusive; or between about 1 ml and about 10 ml, inclusive; or between about 10 ml and about 50 ml, inclusive, of sterile water, or saline solution.

Typically, d-MAPPS™ regenerative biologics platform technology (“d-MAPPS”) is packaged into sterile dosage units which can be stored and distributed for use by attending physicians. Lyophilized or fluid formulations can be in the form of sterile packaged ampule ready for use. A filled ampoule contains a formulation of d-MAPPS. This is generally in a pharmaceutically acceptable carrier and buffered for human use to a pH of about 3.5-10.0, preferably about pH 6.0-7.5. In some embodiments, the formulations are free of preservative where preservatives may exert opposite effects to that required by the formulation. Water or saline solution is used to provide the carrier.

Generally, volumes used here refer to freshly processed, d-MAPPS i.e. 1×strength without any dilution or concentration. In some embodiments, the volumes for use with a nebulizer need to be adjusted/increased to match the amount of active ingredients in the amniotic fluid formations. In particular, if the formulations were stored for a long period of time, where active ingredients (amniotic factors) have deteriorated over time, it is important to volumes for use with a nebulizer are adjusted. In some cases, where lyophilized amniotic fluid formulations are used, these volumes refer to the volume of fluid when the lyophilized powder is reconstituted with the initial volume of sterile water i.e. 1× strength.

The d-MAPPS formulations and d-MAPPS pharmaceutical compositions can be administered in concentrated form, diluted with sterile water, saline or buffer, preferably in the form of aerosol. The formulation may also include additional therapeutic, prophylactic or diagnostic agents. Said agent may be in-mixed with the formulations or mixed in separate containers to be used in conjunction with d-MAPPS formulations. The efficacy of administration is determined by physician evaluations, patient self-evaluations, imaging studies and quality of life evaluations. In some embodiments, d-MAPPS can be administered locally or systemically.

One or more tonicity adjusting agents may be added to provide the desired ionic strength. Tonicity-adjusting agents for use include those which display no or only negligible pharmacological activity after administration. Both inorganic and organic tonicity adjusting agents may be used. Compositions can also include excipients and/or additives. Examples of excipients are surfactants, stabilizers, complexing agents, antioxidants, or preservatives which prolong the duration of use of the finished pharmaceutical formulation, flavorings, vitamins, or other additives known in the art. Complexing agents include, but are not limited to, ethylenediaminetetraacetic acid (EDTA) or a salt thereof, such as the disodium salt, citric acid, nitrilotriacetic acid and the salts thereof. In one embodiment, the complexing agent is EDTA. Preservatives include, but are not limited to, those that protect the solution from contamination with pathogenic particles, including benzalkonium chloride or benzoic acid, or benzoates such as sodium benzoate. Antioxidants include, but are not limited to, vitamins, provitamins, ascorbic acid, vitamin E or salts or esters thereof.

In some embodiments, lyophilized d-MAPPS formulations are used to prevent or treat cancer or tumors in a subject. In some embodiments, lyophilized d-MAPPS is reconstituted by adding an initial volume of water. In other embodiments, the formulation is further diluted to from about 1% to about 99% of the reconstituted d-MAPPS. The refrigerated formulation is readily diluted to from about 1% to about 99% of the original d-MAPPS concentration to a desired concentration for administration. In other embodiments, to minimize the amount of time a patient is required to be confined to a nebulizer, a concentrated formulation may be used to deliver the same effective dosage in a shorter period. In one embodiment, the lyophilized d-MAPPS is reconstituted by adding half of the initial volume of water to achieve amniotic factors administered to a subject at twice the concentration. In a further embodiment, the lyophilized d-MAPPS is reconstituted by adding 10% of the initial volume of water to achieve 10-fold more concentrated solution. In some embodiments, the refrigerated d-MAPPS can be used to reconstitute the lyophilized d-MAPPS to obtain a more concentrated solution.

The d-MAPPS formulations can be administered as frequently as necessary and appropriate. The frequency generally depends on the severity of the cancer/tumor condition, and the responsiveness of the target tissues to the treatment with d-MAPPS formulations. In some embodiments, the d-MAPPS formulations are administered on a once-a-week basis. In other embodiments, the d-MAPPS formulations are administered on a once-a-month basis. In some embodiments, the administration routine can change based on a practitioner's assessment of the patient following an initial treatment. As there is no toxicity known to be associated with the d-MAPPS formulation, it can be injected as often as the physician chooses, unlike steroids that can only be injected infrequently, typically two to three times a year. This feature also represents a significant benefit relative to chemotherapeutic treatments, the administration of which is limited by toxicity and often itself induces tissue damage.

The present invention will be further understood by reference to the following non-limiting examples.

### EXAMPLES

### Example 1

Preparation and Storage of d-MAPPS

Sterile d-MAPPS™ regenerative biologics platform technology (“d-MAPPS”) is an engineered biological product obtained from dMSCs, preferably placental tissue-derived mesenchymal stem cells (“PL-MSCs”), and amniotic fluid of healthy human donors. PL-MSC were grown in complete DMEM. Low passage (<5) PL-MSCs were grown to 60%-80% confluence in multiflasks before isolation. Fresh PL-MSC media were layered and collected after 48 to 72 h (conditioned medium). Exosomes (“Exos”) were isolated by the ultracentrifugation protocol (100,000 g at 4° C. for 70 min). The isolation of exosomes was performed by positive selection using the μMACS™ Separator (Miltenyi Biotec, Bergisch Gladbach, Germany; Cat. No. 130-042-602) and the Exosome Isolation Kit Pan, human (Miltenyi Biotec, Bergisch Gladbach, Germany; Cat. No. 130-110-912) which contained a cocktail of MicroBeads conjugated to the tetraspanin proteins CD9, CD63, and CD81.

For the in vivo characterization of d-MAPPS in a breast cancer model, human placental tissue and amniotic fluid was collected from healthy human donor caesarean sections, as described above. Amniotic fluid and placental tissue were stored in refrigerated condition at 2.5° C. to 6.5° C. prior to the clarification and filtration process. Regarding amniotic fluid preparation, amniotic fluid was centrifuged at 5,000 to 10,000 rpm for 20 minutes to 1 hour in 50 mL to 250 mL receptacles. The supernatant was collected. When collecting the supernatant, it was important to avoid detaching or aspirating insoluble components. If the supernatant contained residual insoluble components, they were pre-filtered with 5 to 10 cellulose ester capsule pre-filters without TRITON® surfactant to avoid contamination in the filtration process. The liquid phase was collected and filtered with poly ether sulfone 1.0 capsule filters and the liquid was collected. The liquid was then filtered with poly ether sulfone 0.25 capsule filter. The filtrate was transferred to vials and sealed with stoppers aseptically. Four samples from the final filtrate were taken to test whether the sterile filtered human amniotic fluid retained exogenous immune cells of interest. Generally, the concentration of the exogenous immune cells in the sterile filtered amniotic fluid was from about 20 pg/mL to about 2400 pg/mL. The concentrations of all the exogenous immune cells in the four samples were in the range of 20-150 pg/mL.

Sterile d-MAPPS for administration to a subject was prepared by collecting amniotic fluid from a subject to produce a sample of raw amniotic fluid. The raw amniotic fluid was then de-cellularized to remove only cells and particulate matter by a series of centrifugation and filtration steps (e.g., centrifuged at 5,000 to 15,000 rpm for 15 minutes to 1 hour; filtration of supernatant with 5 to 10 cellulose ester capsule filters) to produce a de-cellularized amniotic fluid. The quantity of solubilized proteins in the de-cellularized amniotic fluid is between 40% to greater than 90% of the raw amniotic fluid (the concentration of the proteins in the de-cellularized amniotic fluid was from about 20 pg/mL to about 2400 pg/mL). Next, the de-cellularized amniotic fluid was incubated at a temperature between 1° C. and 20° C., or between 2° C. and 9° C., for a period of time effective to increase the quantity of one or more immunostimulatory molecules relative to the raw amniotic fluid comprises placing the de-cellularized amniotic fluid in a sterile vessel at a temperature from 1° C. to 20° C., from 2° C. to 8° C., or 4° C., for one or more days, weeks, months, or up to a year. As described above, said immunostimulatory molecules may include the IL-12 cytokine family (e.g. IL-12, IL-23, IL-27 and IL-35), CSC chemokines (e.g., CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16, and/or CXCL17), and other immunostimulatory molecules know in the art. Notably, when collecting the supernatant, it was important to avoid detaching or aspirating insoluble components. If the supernatant contained residual insoluble components, they were pre-filtered with 5 to 10 cellulose ester capsule pre-filters without TRITON® surfactant to avoid contamination in the filtration process.

Before d-MAPPS isolation, blood samples of healthy donors were tested by laboratories certified under the Clinical Laboratory Improvement Amendments (CLIA) and found negative using United States (U.S) Food and Drug Administration (FDA) licensed tests for detection of: Hepatitis B Virus, Hepatitis C Virus, Human Immunodeficiency Virus Types 1/2, Treponema Pallidum. All of d-MAPPS samples contain dMSCs, AF-MSC Exosomes, and AF-MSC-derived cytokines and growth factors, manufactured under current Good Manufacturing Practices (cGMP), regulated and reviewed by the FDA.

The sterile d-MAPPS prepared according to the above was either frozen at −85° C. or refrigerated at 2-8° C. for two weeks prior to use in the below-described animal study. In some cases, raw amniotic fluid directly harvested from a woman without any centrifugation or filtration steps was used as a control.

### Example 2

d-MAPPS Administration in a Breast Cancer Model

This example describes the in vivo characterization of d-MAPPS™ regenerative biologics platform technology (“d-MAPPS”) in a breast cancer model. A mouse model of breast cancer (BALBc female mice injected with 4T1 mammary carcinoma cells) was used to characterize the antitumor efficacy of d-MAPPS. Tumor burden, mouse survival, tumor weight, and tumor volume associated with d-MAPPS treatment were measured.

Animals

BALB/c mice, eight to ten weeks old, average weight 22-22.5 g, were used in this study. All female mice were nulliparous and non-pregnant. Mice were maintained in animal breeding facilities of the Faculty of Medical Sciences, University of Kragujevac, Serbia. All procedures were performed in accordance with the guidelines for the Principles of Laboratory Animal Care and the Guide for the Care and Use of Laboratory Animals, and all animals received humane care according to the criteria outlined in the Guide for the Care and Use of Laboratory Animals (National Institutes of Health publication 86-23, 1985 revision). All experiments were approved by the Animal Ethical Review Board of the Faculty of Medical Sciences, University of Kragujevac, Serbia. Mice were housed in a temperature-controlled (e.g., temperature of 23+1/−° C. and relative humidity of 62+/−3%) and were administered with standard laboratory chow and water ad libitum. At least eight mice per group were used in each experiment. Prior to d-MAPPS application, all mice have been acclimated to laboratory conditions for 7 days and evaluated for any visual signs of disease or injury. All mice were fed with standard laboratory chow and were provided water ad libitum. Beddings were used after sterilization by autoclaving and changed every three days.

Cells

Mouse mammary carcinoma 4T1 cell line which is syngeneic to the BALB/c mice was purchased from the American Type Culture Collection (ATCC, USA). 4T1 cells were grown in Dulbecco's-Modified Eagles Medium (DMEM) medium supplemented with 10% fetal bovine serum (FBS), 2 mmol/L 1-glutamine, 1 mmol/L penicillin-streptomycin, 1 mmol/L mixed nonessential amino acids (Sigma, USA). Subconfluent monolayers, in log growth phase, were harvested by brief treatment with 0.25% trypsin and 0.02% EDTA in phosphate-buffered saline (PBS, PAA Laboratories GmbH) and washed three times in serum-free PBS before use in vivo.

Experimental Design

Animals were randomly divided into control and experimental groups (n=16 mice per experimental group and n=8 per control group). Mice from experimental groups were inoculated with 5×10⁴ 4T1 tumor cells orthotopically into the fourth mammary fat pad and immediately after were randomly divided in the two groups to receive saline (0.1 mL/intraperitoneal injection (ip.)/day; 4T1+saline-treated mice) or d-MAPPS (4T1+d-MAPPS; 0.1 mL/ip./day). Mice from control groups receive only saline or d-MAPPS (0.1 ml/ip./day). At the end of experiment, 31 days after initial d-MAPPS or saline administration, all mice were killed, blood was collected for biochemical analysis and complete necropsy was performed. The criteria of the gross pathological examination of brain, lungs, stomach, heart, kidney, liver, ovary, testis, small and large intestine were based on the shape, size, color, and consistency of these internal organs. These organs were evaluated primarily to quantify the extent of metastasis.

Estimation of Breast Cancer Growth and Progression

Thirty-six days after tumor cell injection, mice were sacrificed and the primary tumors were surgically removed. The size of primary tumors was assessed by using electronic calipers. Tumor volume was calculated with the following formula: V=4/3π*a/2*b/2*c/2 (a=length, b=width, and c=thickness). Specimens of lungs, liver, and brain tissue were embedded in paraffin, stained with hematoxylin and eosin (H&E) and reviewed in blinded manner by pathologist to confirm the presence of metastatic colonies.

Isolation of Tumor-Infiltrating Leukocytes

By using scissors, the primary breast tumors were minced, until all large sections were processed into 1-2 mm pieces which are digested in 5 mL of DMEM containing 1 mg/mL collagenase I, 1 mM EDTA, and 2% FBS (all from Sigma-Aldrich, Munich, Germany). After incubation for 2 hours at 37° C., 10 mL of 0.25% trypsin was added and incubated for 3 min followed by DNase I (Sigma-Aldrich) solution for 1 min and digests filtered through 40-mm nylon cell strainer (BD Biosciences). Single-cell suspensions were then processed for flow cytometry analysis.

Histopathological Analysis

At the time of necropsy, internal organs (brain, heart, lungs, liver, stomach, gonads, intestines, and kidneys), were removed and collected for microscopic examination to evaluate the extent of metastasis. For this purpose, isolated organs were fixed in 10% formalin, embedded in paraffin, and consecutive 4 μm tissue sections were mounted on slides. Sections were stained with hematoxylin and eosin (H&E) and examined under low-power (100×) light microscopy-equipped digital camera (Zeiss Axioskop 40, Jena, Germany).

Statistical Analysis

Data were expressed as the mean±standard error of the mean (SEM) for each group. The homogeneity of variance and normality distribution were tested with Levene's and Kolmogorov-Smirnov tests, respectively. Results were analyzed by independent Student's T Test. Statistical analyses were performed using SPSS 23.0 for Windows software (SPSS Inc., Chicago, Ill.). The difference was considered significant when p<0.05.

Results

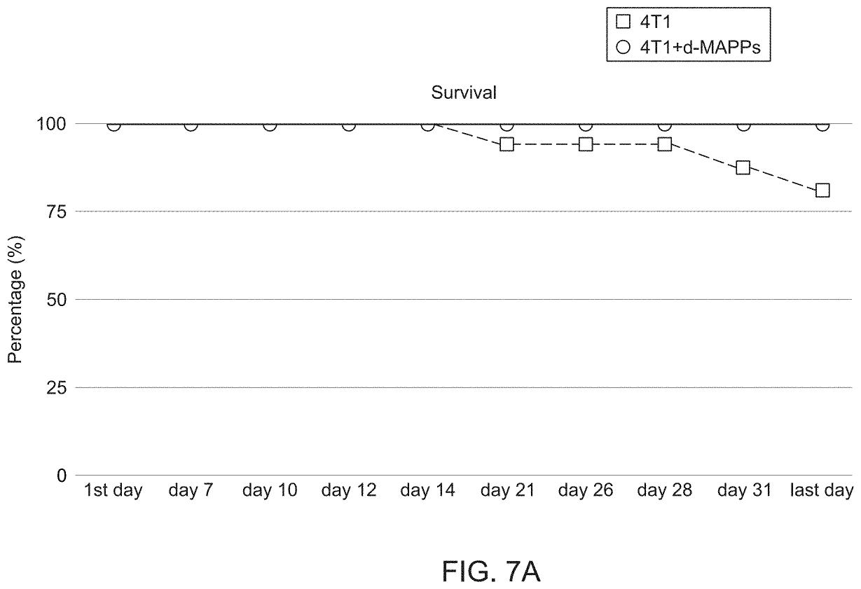

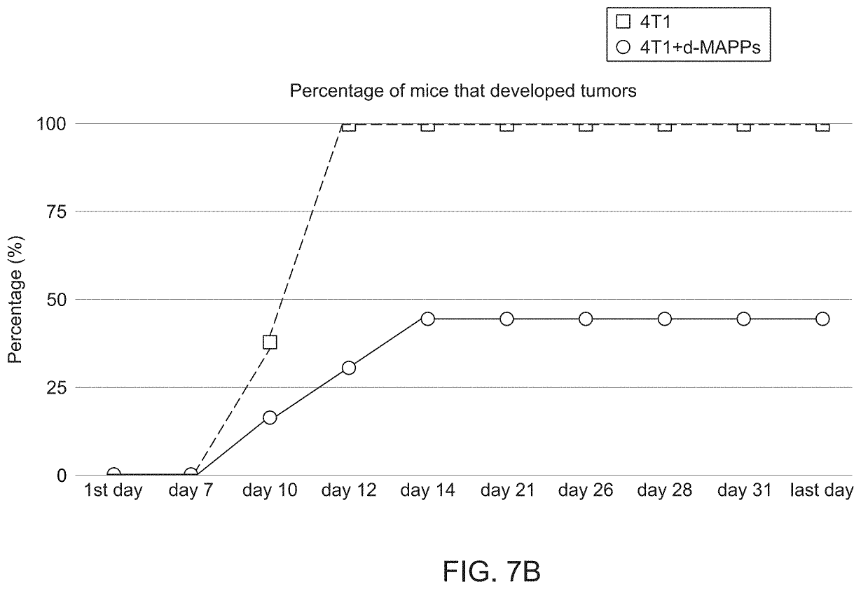

Results indicate that d-MAPPS prevented development of breast cancer in the majority of 4T1-treated mice. Specifically, 43.75% of 4T1+d-MAPPS-treated mice (7 out of 16) developed tumors, while 56.25% of 4T1+d-MAPPS-treated mice (9 out of 16) did not develop tumors. All, 100% (16 out of 16) of 4T1+saline treated mice developed tumors. Thus, d-MAPPS both delayed and decreased the incidence of tumor development (FIG. 7A) in 4T1 d-MAPPS-treated mice. As shown in FIG. 7B, d-MAPPS also significantly improved survival of 4T1 d-MAPPS-treated mice. Specifically, all subjects, 100% (16 out of 16) 4T1+d-MAPPS-treated mice, survived until the end of experiment, while 8 1.25% (13 out of 16) of 4T1-treated mice (e.g., saline only) survived until the end of experiment.

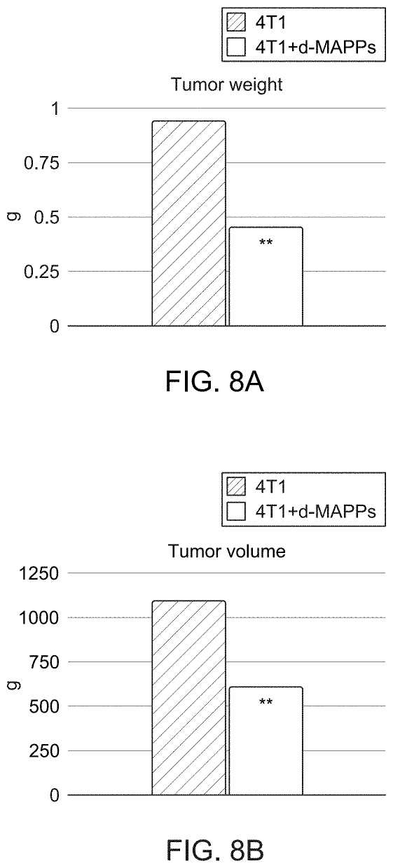

As shown in the line graph of FIG. 7A, a more than fifty percent decrease in the incidence of cancers was observed for the d-MAPPS experimental group. Based on this observation, the present inventors predict that administration of an effective amount of d-MAPPS to any mammalian subject may serve to decrease the incidence of cancer in the mammalian subjects. As depicted in FIG. 7B, improved survival of d-MAPPS-treated mice was also observed relative to saline-treated mice in a 4T1 breast cancer model. Accordingly, the present inventors contemplate that administering an effective amount of d-MAPPS to any mammalian subject with breast cancer, as either a preventative measure or cancer/tumor treatment, may improve survival in the subject. FIG. 8A is a bar graph showing that d-MAPPS significantly reduces tumor weight in subjects (e.g., mice) that developed tumors. FIG. 8B is a bar graph showing that d-MAPPS significantly reduces tumor volume in mice that developed tumors. Accordingly, the present inventors contemplate that administering an effective amount of d-MAPPS to any mammalian subject (e.g., a human, mouse, aquatic mammal, and the like) will significantly reduce tumor weight and tumor volume in the subject.

Regarding the d-MAPPS™ regenerative biologics platform technology (“d-MAPPS”) breast cancer histology analysis, internal organs (brain, heart, lungs, liver, stomach, gonads, intestines, and kidneys), were removed and collected for microscopic examination to evaluate the extent of metastasis. More than 1000 images/potential metastatic fields in mice liver, brain and lungs were analyzed. Key qualitative differences were detected between the experimental groups, showing that d-MAPPS induces apoptosis of breast cancer cells. Analysis of liver cells are provided as an exemplary case. For example, in d-MAPPS treated 41T mice where cancer had metastasized to the liver, there were signs of both decreased tumor volume and local inflammation in the tumor sections of livers of d-MAPPS treated animals relative to the 41T-treated mice. Correspondingly, in untreated d-MAPPS mice (e.g., non-41T mice, treated with d-MAPPS), histological analysis of liver tissue samples confirmed that d-MAPPS did not cause injury to hepatocytes. Specifically, stained liver tissue sections of d-MAPPS treated mice showed normal liver architecture with a lobular organization. Portal areas, containing elements of the hepatic triad (small branches of the portal vein, hepatic artery and bile duct) and lymphatic vessels were distributed along with central venules, providing evidence of a normal, preserved lobular structure of d-MAPPS treated livers. Liver cells were arranged in plates or cords, and were seen radiating from the regions of central venules, extending to the portal areas. Importantly, there were no metastatic colonies in the brain, but they were present in the liver and in the lungs of mice that developed primary tumors. Further, no difference was found in the extent of metastatic colonies between lungs and liver of saline and dMAPPs treated tumor bearing mice. Notably, there was no significant differences in the expression of inflammation-related genes (TNF-alpha, IL-6 and TGF-β) in the livers of control d-MAPPS mice. In order to confirm that d-MAPPS treatment did not cause apoptotic cell death of hepatocytes, expression of apoptosis-related genes was determined in the livers of d-MAPPS treated mice. Significantly, there was no difference in the expression of Bim, Pten, Puma, Noxa, Bak1, Bcl-2, Xiap in the livers of experimental and control mice 7 and 28 days after d-MAPPS administration, confirming that d-MAPPS did not induce apoptosis of hepatocytes in untreated d-MAPPS mice (e.g., non-41T mice, treated with d-MAPPS).

### Example 3

### Serological Assay (Enzyme-Linked Immunosorbent Assay)

This example describes the further in vivo characterization of d-MAPPS™ regenerative biologics platform technology (“d-MAPPS”) in a breast cancer model. A mouse model of breast cancer (BALBc mice injected with 4T1 mammary carcinoma cells) was used to characterize the antitumor efficacy of d-MAPPS. Serological responses of a wide variety of immune molecules associated with d-MAPPS treatment were measured by conventional ELISA assay.

Cytokine and Chemokine Measurement in Serum Samples and Tumor Tissues

Mice intraperitoneally received d-MAPPS (experimental group) or saline (control group), every day for 36 days, as described above. At the end of the experiment, 36 days after initial d-MAPPS or saline administration, all mice were killed, and tumor tissues were collected for ELISA analysis. Tumor tissues were homogenized in cold PBS, and then sonicated for 10 min. Homogenates were centrifuged at 15,000 rpm for 10 min at 4° C., and the total protein concentrations in supernatants were measured using a BCA protein assay kit (Pierce Biotechnology). The content of various exogenous immune cells of interest (e.g., human CXCL16, IL-27 and mouse IL-17, IFN-γ, IL-17, TGF-β and IL-10 content) in the supernatants were determined by commercial enzyme-linked immunosorbent assay (ELISA) sets (R&D Systems, Minneapolis, Minn., USA), according to the manufacturer's instructions. For the determination of cytokine and chemokine serum concentration, blood was obtained from abdominal aorta of mice from experimental and control groups. Serum levels of exogenous immune cells of interest (e.g., human CXCL16, IL-27 and mouse, IFN-γ, IL-17, TGF-β and IL-10 were measured using commercial ELISA sets (R&D Systems, Minneapolis, Minn., USA), according to the manufacturer's instructions.

Intracellular Staining of Tumor-Infiltrating Leukocytes and Flow Cytometry Analysis

Tumor-infiltrating leukocytes were investigated for different cell surface and intracellular markers with flow cytometry. Briefly, 1×10⁶ cells were incubated with anti-mouse F4/80, CD11c, NK1.1, CD80, CD86, I-A, granzyme B, CD178, CD4 and CD8 monoclonal antibodies conjugated with fluorescein isothiocyanate (“FITC”), phycoerythrin (“PE”), peridinin chlorophyll protein (“PerCP”), or allophycocyanin (“APC”) (all from BD Biosciences, San Jose, Calif., USA) following the manufacturer's instructions. For intracellular cytokine staining, cells were stimulated with 50 ng/mL Phorbol 12-myristate 13-acetate (“PMA”), 500 ng/mL ionomycin for 5 h, and GolgiStop (BD Biosciences) was added. Cells were then incubated in a BD fixation/permeabilization solution (BD Cytofix/Cytoperm™ Fixation/Permeabilization Kit) for 20 min at 4° C. Afterwards, cells were washed two times in 1×BD Perm/Wash™ buffer (BD Cytofix/Cytoperm™ Fixation/Permeabilization Kit) and pelleted. Fixed/permeabilized cells were concomitantly resuspended in 50 μL of BD Perm/Wash™ buffer containing a predetermined optimal concentration of fluorochrome-conjugated antibodies specific for TNF-α, IFN-γ, IL-12, IL-4, IL-17, IL-10, FoxP3 by using appropriate anti-mouse monoclonal antibodies conjugated with FITC, PE, PerCP, and APC (BD Biosciences, San Jose, Calif., USA). Cells were incubated with fluorochrome-conjugated antibodies at 4° C. for 30 minutes in the dark. Afterwards, cells were washed 2 times with 1×BD Perm/Wash™ buffer and resuspended in a staining buffer prior to flow cytometric analysis. Flow cytometric analysis was conducted on a BD Biosciences' FACSCalibur and analyzed by using the Flowing Software analysis program.

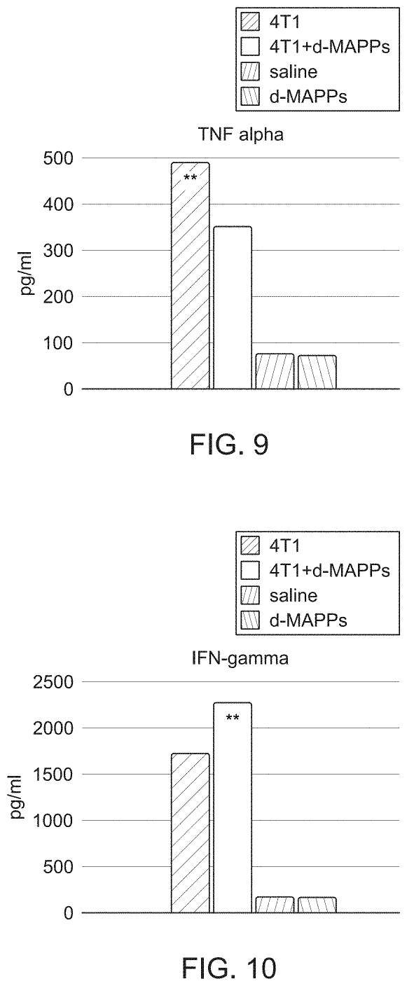

As shown in FIGS. 9-13, ELISA analysis of various endogenous immune cells expressing different cell surface and intracellular markers were carried out, including tumor-infiltrating leukocytes expressing tumor necrosis factor alpha (“TNF-alpha”), interleukin (IL)-17, interleukin (IL)-10, IFN-gamma, and transforming growth factor beta (“TGF-β”). ELISA analysis was carried out on a variety of additional immune cells/markers, wherein cells were measured both in their native state and as related to a murine breast cancer model. These cells included: tumor-infiltrated leukocytes, tumor-infiltrated F4/80+ macrophages, CD80+ cells in tumor-infiltrated F4/80+ macrophages, tumor-infiltrated CD80+F4/80+ macrophages, CD86+ cells in tumor-infiltrated F4/80+ macrophages, CD80+CD86+ cells in tumor-infiltrated F4/80+ macrophages, I-A+ cells in tumor-infiltrated F4/80+ macrophages, tumor-infiltrated I-A+F4/80+ macrophages, IL-12+F4/80+ macrophages, IL-10+ cells in tumor-infiltrated F4/80+ macrophages, IL-10+F4/80+ macrophages, tumor-infiltrated CD11c+ dendritic cells (“DC”), tumor-infiltrated CD11c+ DCs, CD80+ cells in tumor-infiltrated CD11c+ DCs, CD86+ cells in tumor-infiltrated CD11c+ DCs, CD80+CD86+ cells in tumor-infiltrated CD11c+ DCs, CD80+CD86+CD11c+ DCs, I-A+ cells in tumor-infiltrated CD11c+ DCs, TNFalpha+ cells in tumor-infiltrated CD11c+ DCs, IL-12+ cells in tumor-infiltrated CD11 c+ DCs, IL-10+ cells in tumor-infiltrated CD11 c+ DCs, tumor-infiltrated CD4+ T cells, IL-10+ cells in tumor-infiltrated CD4+ T cells, tumor-infiltrated CD4+IL-10+ T cells, FoxP3+ cells in tumor-infiltrated CD4+ T cells, tumor-infiltrated CD4+FoxP3+ T regulatory (Treg) cells, TNFalpha+ cells in tumor-infiltrated CD4+ T cells, tumor-infiltrated CD4+ TNFalpha+ T cells, IL-4+ cells in tumor-infiltrated CD4+ T cells, tumor-infiltrated CD4+IL-17+ T cells, IFN-γ+ cells in tumor-infiltrated CD4+ T cells, tumor-infiltrated CD4+IFN-γ+ T cells, tumor-infiltrated CD8+ T cells, IL-10+ cells in tumor-infiltrated CD8+ T cells, tumor-infiltrated CD8+IL-10+ T cells, FoxP3+ cells in tumor-infiltrated CD8+ T cells, tumor-infiltrated CD8+FoxP3+ regulatory cells, TNFalpha+ cells in tumor-infiltrated CD8+ T cells, tumor-infiltrated CD8+ TNFalpha+ T cells, IL-4+ cells in tumor-infiltrated CD8+ T cells, tumor-infiltrated CD8+IL-4+ Tcells, IL-17+ cells in tumor-infiltrated CD8+ T cells, tumor-infiltrated CD8+IL-17+ T cells, IFN-γ+ cells in tumor-infiltrated CD8+ T cells, tumor-infiltrated CD8+IFN-y+ T cells, CD178+ cells in tumor-infiltrated CD8+ T cells, tumor-infiltrated CD8+CD178+ T cells, Granzyme B+ cells in tumor-infiltrated CD8+ T cells, tumor-infiltrated CD8+Granzyme B+ T cells, tumor-infiltrated NK1.1+NK cells, tumor-infiltrated NK1.1+NK cells, CD178+ cells in tumor-infiltrated NK1.1+NK cells, tumor-infiltrated NK1.1+CD178+NK cells, Granzyme B+ cells in tumor-infiltrated NK1.1+NK cells, tumor-infiltrated NK1.1+Granzyme B+NK cells, IL-17+ cells in tumor-infiltrated NK1.1+NK cells, tumor-infiltrated NK1.1+IL-17+NK cells, IFN-y+ cells in tumor-infiltrated NK1.1+NK cells, and tumor-infiltrated NK1.1+IFN-γ+NK cells.

Statistical Analyses

The data were analyzed using statistical package SPSS, version 21. The normality of distribution was tested by the Kolmogorov-Smirnov test. The results were analyzed using the Student t-test. All data in this study were expressed as the mean±standard error of the mean (SEM). Values of p<0.05 were considered statistically significant.

Results

In brief, administratino of d-MAPPS augmented T cell-driven immune response to murine mammary carcinoma by enhancing DC-based generation of Th1 and Th17 cells and by increasing cytotoxicity of CD8+CTLs in a CXCL16 and IL-27-dependent manner.

1. D-MAPPS Enhanced T Cell-Driven Immune Response to Murine Mammary Carcinoma

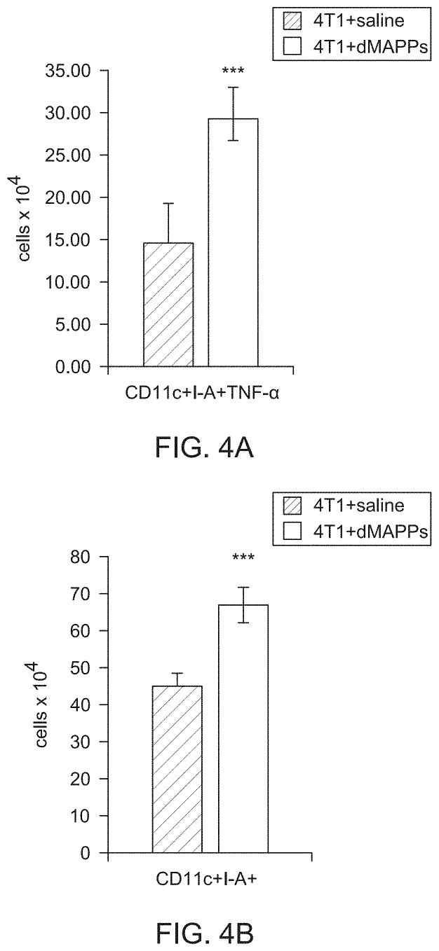

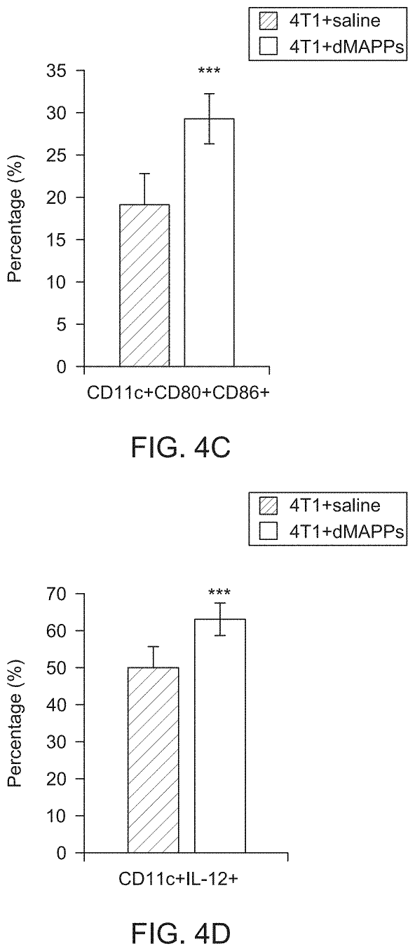

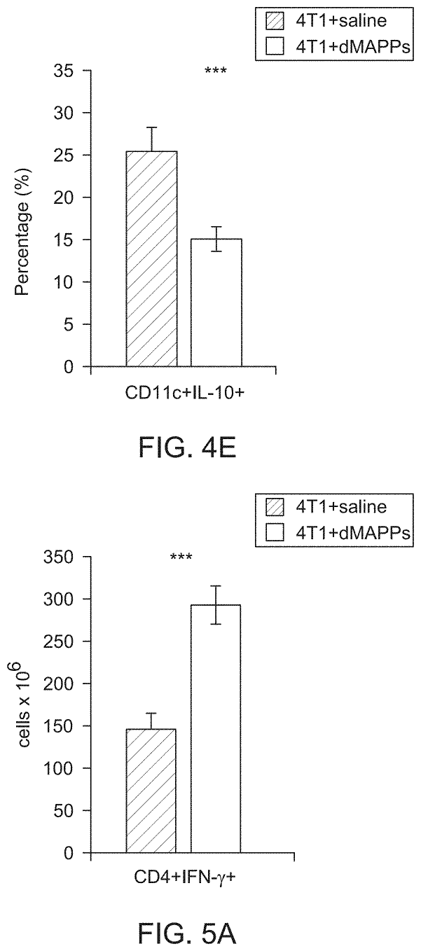

Administration of d-MAPPS induce increased antigen-presenting activity of tumor-infiltrated DCs (FIG. 4). As a result, a significantly higher number of anti-tumorigenic CD4+ Th1 and Th17 cells were observed in the mammary cancers of 4T1+d-MAPPS treated mice (FIG. 5A-C). Total numbers of IFN-γ-producing Th1 and IL-17-producing Th17 cells were also significantly increased in the breast tumors of 4T1+d-MAPPS treated animals (FIG. 5A-C; p<0.001) while there was no significant difference in total number of CD4+IL-4+ Th2 cells between the tumors of 4T1+saline and 4T1+d-MAPPS treated mice (FIG. 5D), confirming that d-MAPPS favored generation of anti-tumorigenic Th1 and Th17 immune response. Moreover, d-MAPPS inhibited generation of immunosuppressive phenotype in tumor-infiltrated CD4+ T cells (FIG. 5E-F). Remarkably decreased number of FoxP3-expressing and IL-10-producing CD4+ Tregs were observed in the tumors of 4T1+d-MAPPS treated animals compared to tumor bearing animals that received saline (FIG. 5E-F). D-MAPPS also significantly increased the presence of tumor-infiltrated cytotoxic CD178-expressing and Granzyme B-producing cytotoxic CD8+ T cells (see CTLs; FIG. 6A-B). Additionally, d-MAPPS favored activation and expansion of tumoricidal IFN-γ-producing and IL-17-producing CD8+ CTLs (FIG. 6C-D) and inhibited generation of immunosuppressive and pro-tumorigenic IL-10-producing and FoxP3-expressing CD8+ T cells (FIG. 6E-F).

2. D-MAPPS Treated Mice Showed Delayed Mammary Tumor Appearance and Slower Tumor Growth by Enhancing Anti-Tumor Immune Response

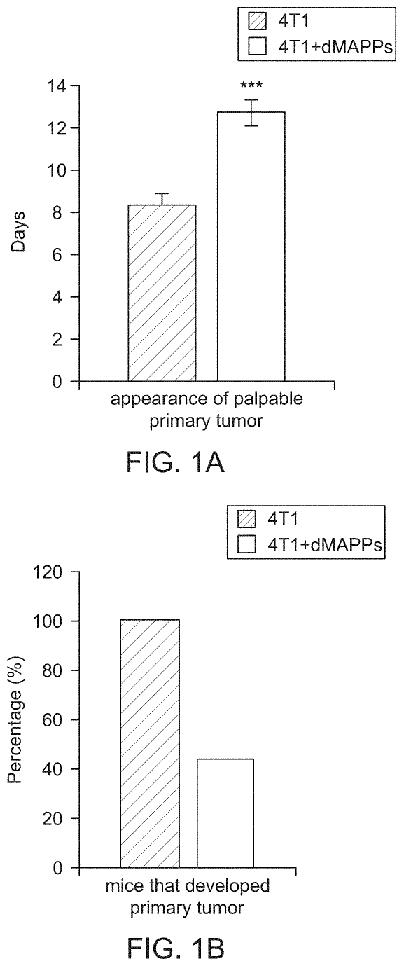

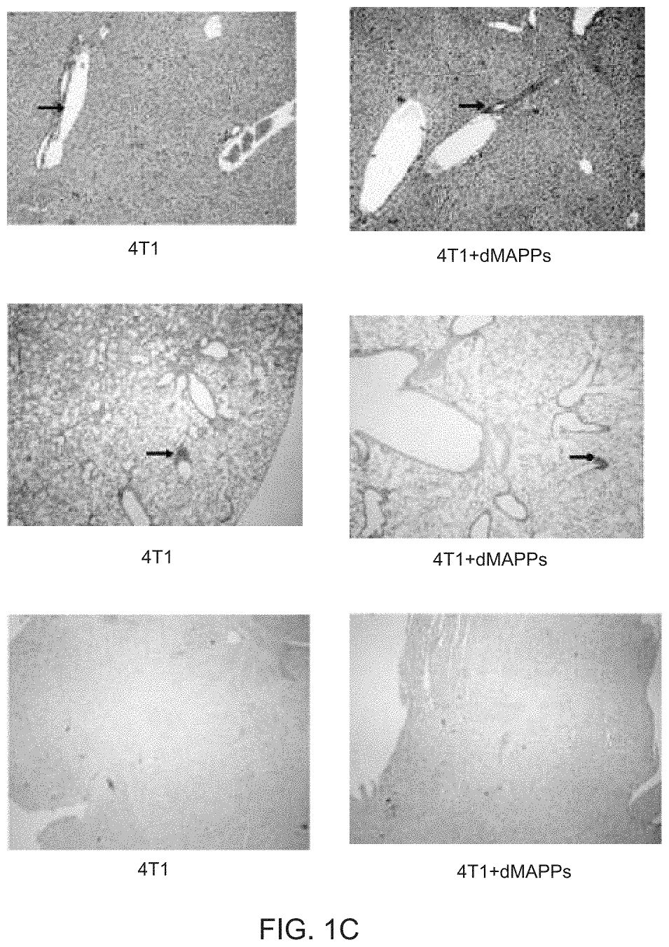

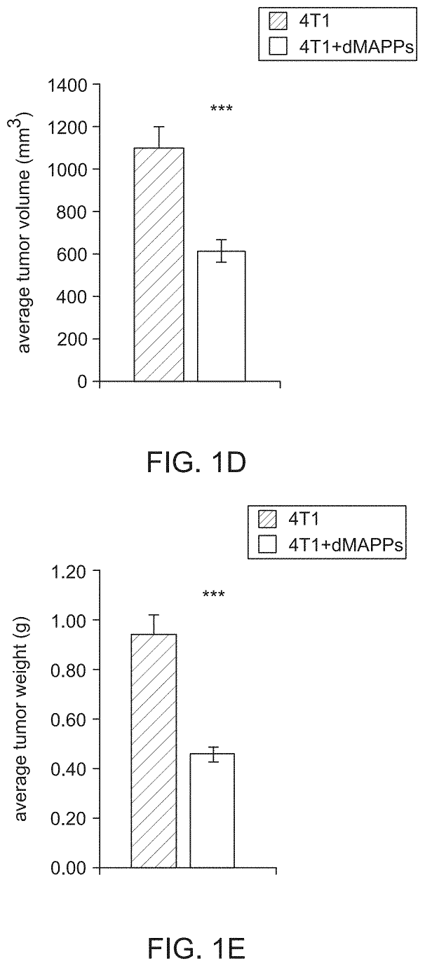

After orthotopical administration, the 4T1 cells intensively divide and rapidly form tumors which become palpable within 8-12 days. As shown in FIG. 1A, d-MAPPS treated mice show delayed mammary tumor appearance. Mean value of time period in days from inoculation of tumor cells to the appearance of palpable primary tumor in 4T1+d-MAPPS+-treated mice was significantly longer than in 4T1+saline-treated animals (mean±SEM: 12.7 days±0.6 versus 8.3 days±0.5, p<0.001).

As described above, d-MAPPS prevented development of breast cancer in majority of 4T1-treated mice. Precisely, 43.75% of 4T1+d-MAPPStreated mice (7 out of 16) developed tumor while all (100%) of 4T1+saline-treated mice developed breast cancer (FIG. 1i). Lung and liver metastatic colonies were determined in all 4T1+saline-treated mice and in 4T1+d-MAPPS treated mice that developed tumors (FIG. 1C upper and middle panels), while brain metastasis were not found either in 4T1+saline or 4T1+d-MAPPstreated tumor bearing mice (FIG. 1C lower panels). Accordingly, d-MAPPS significantly improved survival of 4T1-treated mice. While 18.75% (3 out of 16) of 4T1+saline-treated died due to the tumor progression and dissemination (FIG. 7A), all of 4T1+d-MAPPStreated mice survived till the end of experiment (day 36). On day 36, the mean value of primary tumor volume in 4T1+saline-treated mice was significantly higher than in 4T1+d-MAPPStreated mice (FIG. 1D). Similarly, primary tumor weight was significantly higher in saline-treated than in dMAPPS treated tumor bearing animals (FIG. 1E).

Importantly, d-MAPPS significantly increased serum levels of anti-tumorigenic immunostimulatory molecules including chemokines and cytokines CXCL16 (FIG. 2A), IL-27 (FIG. 2B), IFN-γ (FIG. 2C), IL-17 (FIG. 2D)) and down-regulated concentration of immunosuppressive cytokines TGF-β (FIG. 2E) and IL-10 (FIG. 2F)) in mice with established mammary tumors. Analogously, increased concentration of CXCL16, IL-27, IFN-γ and IL-17 and decreased concentration of TGF-3 and IL-10 were measured in the tumors of 4T1+d-MAPPStreated mice (FIG. 2G), indicating that modulation of anti-tumor immune response was mainly responsible for d-MAPPS dependent suppression of mammary cancer growth.

The most dramatic increase in immunostimulatory molecule was observed in the 2-9° C. incubation group (particularly IL-27 and CXCL16). These data were surprising and are not predicted from prior experiments and literature in the field. For example, it was surprising that the step of incubating de-cellularized amniotic fluid at a temperature between 1° C. and 20° C., or between 2° C. and 9° C., resulted in an increase in the quantity of one or more immunostimulatory molecules (e.g. IL-17, IL-27, and CXCL16) relative to the raw amniotic fluid, and that said increased quantities of said immunostimulatory molecules induced down-regulation of serum levels of immunosuppressive IL-10 and TFG-β in the tumor microenvironment. The present inventors therefore disclose a de-cellularized human amniotic fluid formulation (d-MAPPS) which retains an unusually high level of immunostimulatory molecules (particularly heterodimeric cytokines and/or CSC chemokines) in the starting materials (e.g., the starting pharmaceutical composition comprising d-MAPPS) while having reduced side effects relative to other anti-cancer agents known in the field (e.g., chemotherapy agents). As described above, this fact renders d-MAPPS to be an ideal adjuvant therapy to be administered in combination with chemotherapeutic agents and the like.

3. D-MAPPS Improved Antigen-Presenting Properties of Tumor Infiltrated Dendritic Cells

As described above, in order to interrogate the contribution of innate immunity in d-MAPPS based modulation of breast cancer growth and progression, phenotype and function of DCs, natural killer (NK) cells and macrophages were analyzed in the tumors of 4T1+saline and 4T1+d-MAPPS treated mice.

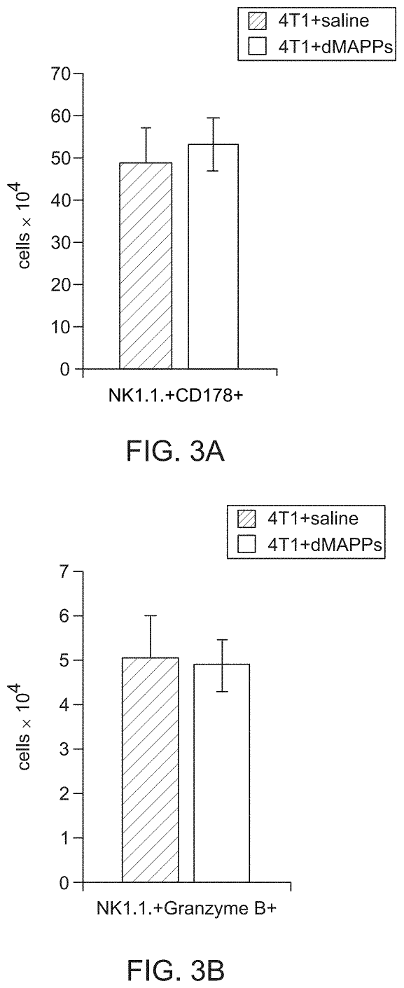

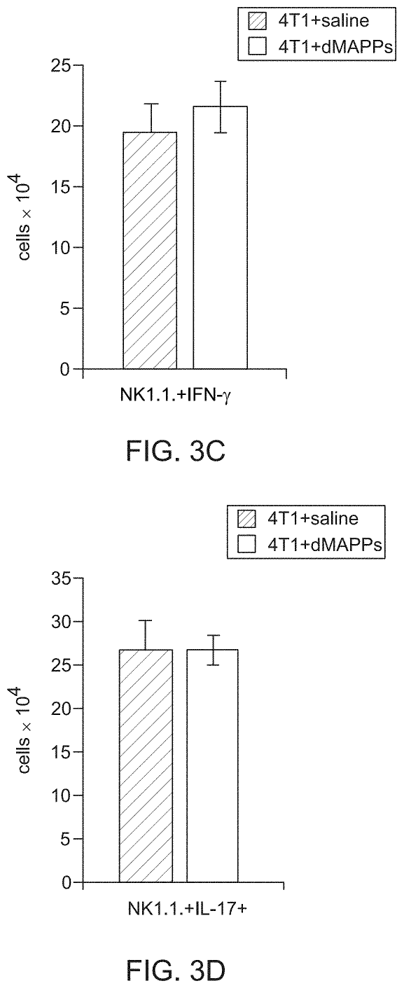

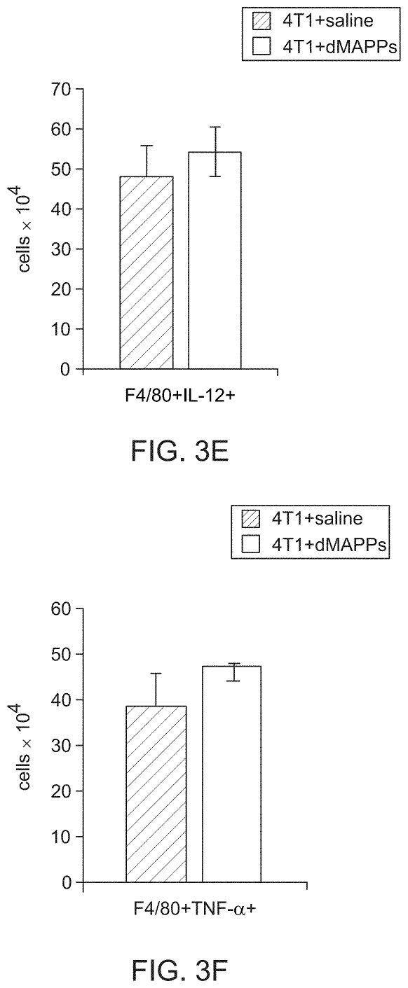

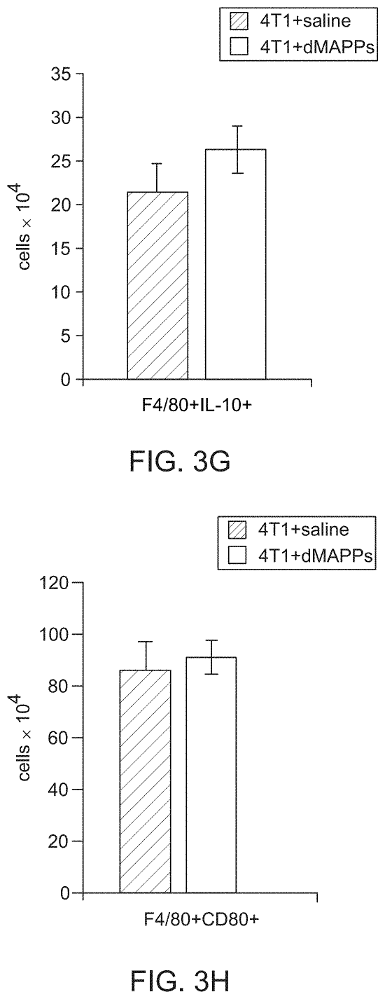

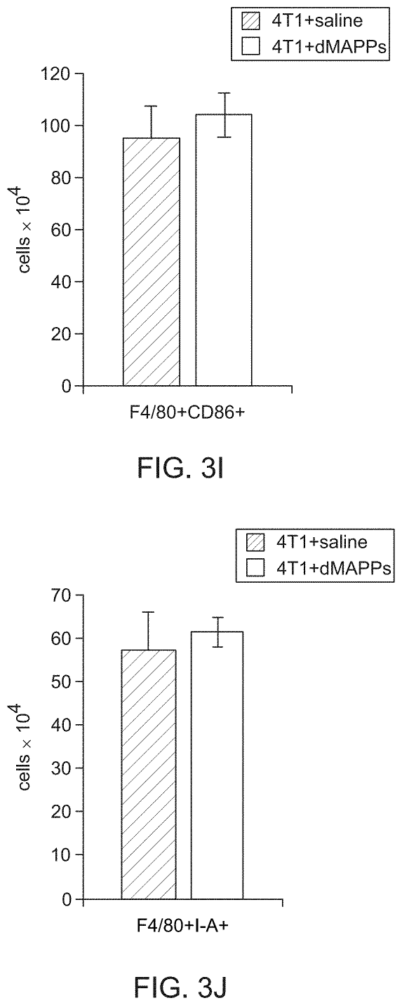

D-MAPPS did not significantly alter phenotype and function of NK cells and macrophages (FIG. 3). There was no significant difference in the total number of tumor infiltrated, cytotoxic CD178 and Granzyme B-expressing, IFN-γ or IL-17-producing NK1.1+NK cells (FIG. 3A-D) and in the total number of IL-12-, TNF-α and IL-10-producing, CD80, CD86 and I-A-expressing F4/80+ TAMs (FIG. 3E-J) between 4T1+saline and 4T1+d-MAPPS treated mice.

Importantly, d-MAPPS significantly enhanced antigen-presenting properties of tumor infiltrated DCs (FIG. 4). Significantly higher number of DCs that express MHC class II molecule and produce TNF-α (FIG. 4A-B) and higher percentage of DCs that express co-stimulatory CD80 and CD86 molecules (FIG. 4C); and produce IL-12 (FIG. 4D) were observed in the tumors of 4T1+d-MAPPStreated mice. Additionally, d-MAPPS reduced percentage of tolerogenic, IL-10-producing DCs in the tumors (FIG. 4E) preventing tumor cell-driven generation of immunosuppressive microenvironment.

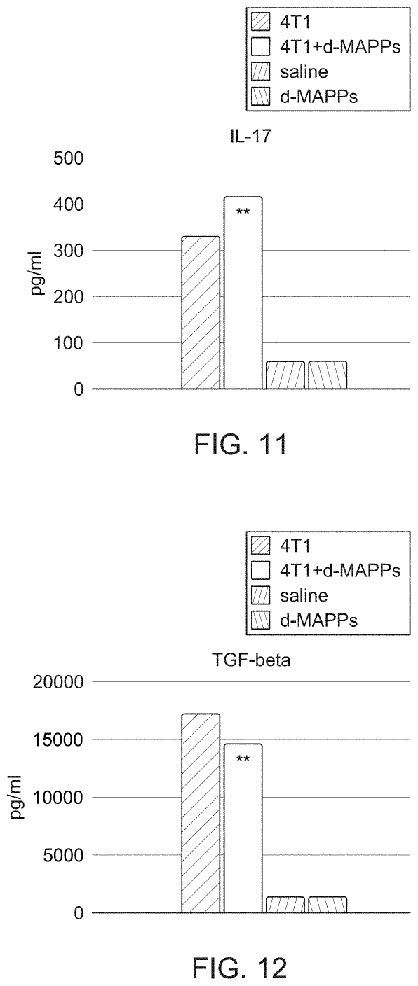

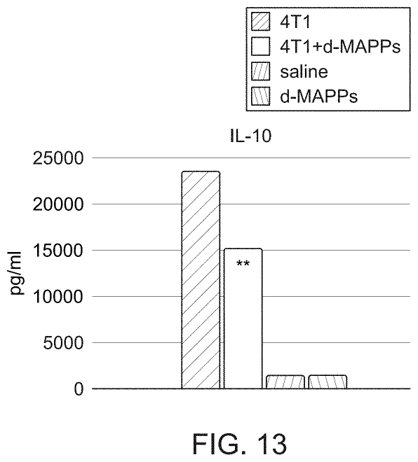

As shown in FIG. 9, d-MAPPS treatment also significantly decreased serum levels of TNF-alpha in a 4T1 breast cancer model. Further, d-MAPPS treatment significantly increased serum levels of anti-tumorigenic IFN-gamma in a 4T1 breast cancer model (FIG. 10). FIG. 11 shows a bar graph indicating that d-MAPPS treatment significantly increased serum levels of IL-17 in a 4T1 breast cancer model. Similarly, FIG. 12 shows that d-MAPPS treatment significantly decreased serum levels of TGF-beta in a 4T1 breast cancer model. Finally, FIG. 13 shows that d-MAPPS treatment significantly decreases serum levels of IL-10 in a 4T1 breast cancer model.

As described above, numerous other endogenous immune cells were measured by ELISA. Similar to the above, results suggested that d-MAPPS may serve as a surprisingly effective preventative therapy for breast cancer given its recruitment of pro-inflammatory molecules to tumor sites. These additional results are summarized as follows: 1) d-MAPPS did not significantly alter total number of tumor-infiltrated leukocytes in murine breast cancer, 2) d-MAPPS did not significantly alter percentage of tumor-infiltrated F4/80+ macrophages in murine breast cancer, 3) d-MAPPS did not significantly alter total number of tumor-infiltrated F4/80+ macrophages in murine breast cancer, 4) d-MAPPS did not significantly alter percentage of CD80+ cells in tumor-infiltrated F4/80+ macrophages, 5) d-MAPPS did not significantly alter total number of tumor-infiltrated CD80+F4/80+ macrophages in murine breast cancer, 6) d-MAPPS did not significantly alter percentage of CD86+ cells in tumor-infiltrated F4/80+ macrophages, 7) d-MAPPS did not significantly alter total number of tumor-infiltrated CD86+F4/80+ macrophages in murine breast cancer, 8) d-MAPPS did not significantly alter percentage of CD80+CD86+ cells in tumor-infiltrated F4/80+ macrophages, 9) d-MAPPS did not significantly alter total number of tumor-infiltrated CD80+CD86+F4/80+ macrophages in murine breast cancer, 10) d-MAPPS did not significantly alter percentage of I-A+ cells in tumor-infiltrated F4/80+ macrophages, 11) d-MAPPS did not significantly alter total number of tumor-infiltrated I-A+F4/80+ macrophages in murine breast cancer, 12) d-MAPPS did not significantly alter percentage of TNFalpha+ cells in tumor-infiltrated F4/80+ macrophages, 13) d-MAPPS did not significantly alter total number of tumor-TNFalpha+F4/80+ macrophages in murine breast cancer, 14) d-MAPPS did not significantly alter percentage of IL-12+ cells in tumor-infiltrated F4/80+ macrophages, 15) d-MAPPS did not significantly alter total number of tumor-infiltrated IL-12+F4/80+ macrophages in murine breast cancer, 16) d-MAPPS did not significantly alter percentage of IL-10+ cells in tumor-infiltrated F4/80+ macrophages, 17) d-MAPPS did not significantly alter total number of tumor-infiltrated IL-10+F4/80+ macrophages, 18) d-MAPPS did not significantly alter percentage of tumor-infiltrated CD11c+ dendritic cells (DC) in murine breast cancer, 19) d-MAPPS did not significantly alter total number of tumor-infiltrated CD11c+ DCs in murine breast cancer, 20) d-MAPPS did not significantly alter percentage of CD80+ cells in tumor-infiltrated CD11c+ DCs, 21) d-MAPPS did not significantly alter total number of tumor-infiltrated CD80+CD11c+ DCs in murine breast cancer, 22) d-MAPPS did not significantly alter percentage of CD86+ cells in tumor-infiltrated CD11c+ DCs, 23) d-MAPPS did not significantly alter total number of tumor-infiltrated CD86+CD11c+ DCs in murine breast cancer, 24) d-MAPPS significantly increased percentage of CD80+CD86+ cells in tumor-infiltrated CD11c+ DCs, 25) d-MAPPS did not significantly alter total number of tumor-infiltrated CD80+CD86+CD11c+ DCs in murine breast cancer, 26) d-MAPPS did not significantly alter percentage of I-A+ cells in tumor-infiltrated CD11c+ DCs, 27) d-MAPPS significantly increased total number of tumor-infiltrated I-A+CD11c+ DCs in murine breast cancer, 28) d-MAPPS did not significantly alter percentage of TNF alpha+ cells in tumor-infiltrated CD11c+ DCs, 29) d-MAPPS significantly increased total number of tumor-infiltrated TNF alpha+CD11c+ DCs in murine breast cancer (e.g., administering d-MAPPS for the treatment of murine breast cancer in a subject increases the total number of tumor-infiltrated RNF alpha dendritic cells in the subject, 30) d-MAPPS significantly increased percentage of IL-12+ cells in tumor-infiltrated CD11c+ DCs, 31) d-MAPPS did not significantly alter total number of tumor-infiltrated IL-12+CD11c+ DCs in murine breast cancer, 32) d-MAPPS significantly decreased percentage of IL-10+ cells in tumor-infiltrated CD11c+ DCs, 33) d-MAPPS did not significantly alter total number of tumor-infiltrated IL-10+CD11c+ DCs in murine breast cancer, 34) d-MAPPS significantly increased percentage of tumor-infiltrated CD4+ T cells in murine breast cancer, 35) d-MAPPS did not significantly alter total number of tumor-infiltrated CD4+ T cells in murine breast cancer, 36) d-MAPPS significantly decreased percentage of IL-10+ cells in tumor-infiltrated CD4+ T cells in murine breast cancer, 37) d-MAPPS significantly decreased total number of tumor-infiltrated CD4+IL-10+ T cells in murine breast cancer, 38) d-MAPPS significantly decreased percentage of FoxP3+ cells in tumor-infiltrated CD4+ T cells in murine breast cancer, 39) d-MAPPS significantly decreased total number of tumor-infiltrated CD4+FoxP3+ T regulatory (Treg) cells in murine breast cancer, 40) d-MAPPS significantly decreased percentage of TNFalpha+ cells in tumor-infiltrated CD4+ T cells in murine breast cancer, 41) d-MAPPS significantly decreased total number of tumor-infiltrated CD4+ TNFalpha+ T cells in murine breast cancer, 42) d-MAPPS did not significantly alter percentage of IL-4+ cells in tumor-infiltrated CD4+ T cells, 43) d-MAPPS did not significantly alter total number of tumor-infiltrated CD4+IL-4+ Tcells in murine breast cancer, 44) d-MAPPS significantly increased percentage of IL-17+ cells in tumor-infiltrated CD4+ T cells in murine breast cancer, 45) d-MAPPS significantly increased total number of tumor-infiltrated CD4+IL-17+ T cells in murine breast cancer, 46) d-MAPPS significantly increased percentage of IFN-y+ cells in tumor-infiltrated CD4+ T cells in murine breast cancer (e.g., administering d-MAPPS for the treatment of murine breast cancer in a subject enhances the total number of IFN-γ+ cells in tumor-infiltrated CD4+ T cells in the subject, 47) d-MAPPS significantly increased total number of tumor-infiltrated CD4+IFN-y+ T cells in murine breast cancer, 48) d-MAPPS did not significantly alter percentage of tumor-infiltrated CD8+ T cells in murine breast cancer, 49) d-MAPPS did not significantly alter total number of tumor-infiltrated CD8+ T cells in murine breast cancer, 50) d-MAPPS significantly decreased percentage of IL-10+ cells in tumor-infiltrated CD8+ T cells in murine breast cancer, 51) d-MAPPS did not significantly alter total number of tumor-infiltrated CD8+IL-10+ T cells in murine breast cancer, 52) d-MAPPS significantly decreased percentage of FoxP3+ cells in tumor-infiltrated CD8+ T cells in murine breast cancer, 53) d-MAPPS did not significantly alter total number of tumor-infiltrated CD8+FoxP3+ regulatory cells in murine breast cancer, 54) d-MAPPS significantly decreased percentage of TNFalpha+ cells in tumor-infiltrated CD8+ T cells in murine breast cancer, 55) d-MAPPS did not significantly alter total number of tumor-infiltrated CD8+ TNFalpha+ T cells in murine breast cancer, 56) d-MAPPS significantly decreased percentage of IL-4+ cells in tumor-infiltrated CD8+ T cells, 57) d-MAPPS did not significantly alter total number of tumor-infiltrated CD8+IL-4+ Tcells in murine breast cancer, 58) d-MAPPS significantly increased percentage of IL-17+ cells in tumor-infiltrated CD8+ T cells in murine breast cancer, 59) d-MAPPS significantly increased total number of tumor-infiltrated CD8+IL-17+ T cells in murine breast cancer, 60) d-MAPPS significantly increased percentage of IFN-y+ cells in tumor-infiltrated CD8+ T cells in murine breast cancer, 61) d-MAPPS significantly increased total number of tumor-infiltrated CD8+IFN-y+ T cells in murine breast cancer, 62) d-MAPPS significantly increased percentage of CD178+ cells in tumor-infiltrated CD8+ T cells in murine breast cancer, 63) d-MAPPS significantly increased total number of tumor-infiltrated CD8+CD178+ T cells in murine breast cancer, 64) d-MAPPS did not significantly alter percentage of Granzyme B+ cells in tumor-infiltrated CD8+ T cells in murine breast cancer, 65) d-MAPPS significantly increased total number of tumor-infiltrated CD8+Granzyme B+ T cells in murine breast cancer, 66)d-MAPPS did not significantly alter percentage of tumor-infiltrated NK1.1+NK cells in murine breast cancer, 67) d-MAPPS did not significantly alter total number of tumor-infiltrated NK1.1+NK cells in murine breast cancer, 68) d-MAPPS did not significantly alter percentage of CD178+ cells in tumor-infiltrated NK1.1+NK cells in murine breast cancer, 69) d-MAPPS did not significantly alter total number of tumor-infiltrated NK1.1+CD178+NK cells in murine breast cancer, 70) d-MAPPS did not significantly alter percentage of Granzyme B+ cells in tumor-infiltrated NK1.1+NK cells in murine breast cancer, 71) d-MAPPS did not significantly alter total number of tumor-infiltrated NK1.1+Granzyme B+NK cells in murine breast cancer, 72) d-MAPPS did not significantly alter percentage of IL-17+ cells in tumor-infiltrated NK1.1+ NK cells in murine breast cancer, 73) d-MAPPS did not significantly alter total number of tumor-infiltrated NK1.1+IL-17+ NK cells in murine breast cancer, 74) d-MAPPS did not significantly alter percentage of IFN-y+ cells in tumor-infiltrated NK1.1+ NK cells in murine breast cancer, and 75) d-MAPPS did not significantly alter total number of tumor-infiltrated NK1.1+IFN-γ+ NK cells in murine breast cancer.

Thus, in vivo characterization of d-MAPPS in a breast cancer model indicates that d-MAPPS may serve as a useful breast cancer prophylactic and treatment in mammalian subjects. This is particularly evident in view of the recruitment of pro-inflammatory molecules and other anti-cancer molecules shown above. By enhancing or inducing anti-cancer immune cells, administration of d-MAPPS serves to abrogate a subject's natural defenses against cancers, tumors, and related disorders.

1. A method for prevention and treatment of cancers and tumors in a subject, comprising:
administering to the subject an effective amount of d-MAPPS.

2. A method for prevention and treatment of cancers and tumors in a subject by altering response of immune cells in the subject, comprising;
administering to the subject an effective amount of d-MAPPS, thereby altering the response of endogenous immune cells in the subject.

3. The method of claim 2, wherein the endogenous immune cells comprise dendritic cells, macrophages, T cells, and/or natural killer cells.

4. The method of claim 3, wherein altering the response of endogenous immune cells in the subject comprises enhancing or inducing endogenous immune cells in the tumor of the subject.

5. The method of claim 1, wherein d-MAPPS is administered in combination with a second anti-cancer therapeutic agent.

6. The method of claim 4, wherein the endogenous immune cells comprise granzyme-expressing lymphocytes.

7. The method of claim 1, wherein d-MAPPS comprises heterodimeric cytokine(s) and/or CSC chemokine(s).

8. The method of claim 1, wherein administering the effective amount of d-MAPPS prevents cancer in the subject and/or decreases the incidence of cancer in the subject.

9. The method of claim 1, wherein administering d-MAPPS reduces tumor weight and/or tumor volume in the subject.

10. The method of claim 1, wherein d-MAPPS is administered systemically or at tumor locations in the subject.

11. The method of claim 1, wherein d-MAPPS is administered in combination with one or more checkpoint inhibitors, thereby preventing exhaustion of T cells in the tumor microenvironment and/or enhancing systemic anti-tumor effects of d-MAPPS.

12. The method of claim 11, wherein checkpoint inhibitors comprise one or more of PD-1, PD-L1 (B7-H1), OX40/OX-40L, CTLA-4, and LAG3.

13. The method of claim 1 wherein d-MAPPS is administered in combination with one or more adjuvants, antigens, excipients, vaccines, allergens, antibiotics, gene therapy vectors, vaccines, kinase inhibitors, co-stimulatory molecules, TLR agonists, or TLR antagonists.

14. The method of claim 1, wherein d-MAPPS is devoid of amniotic stem cells, elements of micronized membrane, and chorion particles.

15. The method of claim 1, wherein the cancer comprises breast cancer or a blood cancer.

16. The method of claim 1, wherein administering d-MAPPS for the treatment of breast cancer enhances or induces tumor-infiltrated TNF alpha dendritic cells in the subject.

17. The method of claim 1, wherein administering d-MAPPS for the treatment of breast cancer enhances IFN-γ cells in tumor-infiltrated CD4 T cells in the subject.

18. The method of claim 10, further comprising administering to a site in need thereof, or administering adjacent to the site in need thereof an effective amount of a sterile de-cellularized filtered non-heat-treated d-MAPPS.

19. The method of claim 18, wherein d-MAPPS is administered as a solution, suspension, or powder.

20. The method of claim 1 wherein d-MAPPS is administered with a pharmaceutically acceptable carrier for injection.

20. (canceled)

21. The method of claim 1, wherein d-MAPPS is administered in combination with one or more therapeutic, prophylactic, or diagnostic agents.

22. The method of claim 1, wherein d-MAPPS is administered in combination with one or more agents selected from the group consisting of dMSCs, antimicrobial agents, analgesic agents, local anesthetic agents, anti-inflammatory agents, anti-oxidant agents, immunosuppressant agents, anti-allergenic agents, enzyme cofactors, essential nutrients, growth factors, and combinations thereof.

23. The method of claim 7, wherein following administration of d-MAPPS, the heterodimeric cytokine IL-27 induces down-regulation of serum levels of immunosuppressive proteins IL-10 and TFG-β.

24. A pharmaceutical composition comprising d-MAPPS and one or more pharmaceutically acceptable excipients.

25. The pharmaceutical composition of claim 24, wherein d-MAPPS comprises one or more immunostimulatory molecules.

26. The pharmaceutical composition of claim 24 further comprising one or more agents selected from the group consisting of adjuvants, antioxidants, anti-inflammatory agents, growth factors, neuroprotective agents, antimicrobial agents, local anesthetics, and combinations thereof.

27. The pharmaceutical composition of claim 24 further comprising one or more exosomes generated ex vivo from mesenchymal stem cells.

28. The pharmaceutical composition of claim 27 wherein the mesenchymal stem cells are placental tissue-derived mesenchymal stem cells.

29. The composition of claim 24, wherein the process by which d-MAPPS is prepared comprises:
(a) collecting placental tissue and amniotic fluid under sterile conditions from a subject to produce a sample of raw amniotic fluid;
(b) de-cellularizing the raw amniotic fluid to remove only cells and particulate matter by a series of centrifugation and filtration steps to produce a de-cellularized amniotic fluid, wherein the quantity of the solubilized proteins in the de-cellularized amniotic fluid is between 40% to greater than 90% of the raw amniotic fluid; and
(c) wherein the step of incubating the de-cellularized amniotic fluid at a temperature between 1° C. and 20° C., or between 2° C. and 9° C., for a period of time effective to increase the quantity of the one or more immunostimulatory molecules relative to the raw amniotic fluid comprises placing the de-cellularized amniotic fluid in a sterile vessel at a temperature from 1° C. to 20° C., from 2° C. to 9° C., or 4° C., for one or more days, weeks, months, or up to a year.

30. The pharmaceutical composition of claim 27 wherein the mesenchymal stem cells are placental tissue-derived mesenchymal stem cells.

31. The composition of claim 29, wherein the amniotic fluid further comprises one or more preservatives.

32. The method of claim 1, wherein d-MAPPS comprises at least 300 human growth factors, wherein said growth factors are present at lower concentrations in d-MAPPS than in human amniotic fluid.
